(19) United States
(12) Patent Application Publication
(10) Pub. No.: US 2024/0336565 A1
(43) Pub. Date: Oct. 10, 2024

(54) SOLID FORMS OF OSANETANT

(71) Applicant: Acer Therapeutics, Inc., Newton, MA (US)

(72) Inventor: John KLOPP, Martinez, CA (US)

(21) Appl. No.: 18/574,284

(22) PCT Filed: Jul. 1, 2022
(86) PCT No.: PCT/US2022/035955

§ 371 Date: Dec. 26, 2023

Related U.S. Application Data
(60) Provisional application No. 63/218,211, filed on Jul. 2, 2021.

(51) Int. Cl. C07D 211/58; A61K 31/4545; C07C 309/04; C07C 309/30; C07C 309/35

(52) U.S. Cl. CPC C07D 211/58; A61K 31/4545; C07C 309/04; C07C 309/30; C07C 309/35

(57) ABSTRACT

Provided herein are solid forms of osanetant, including salts thereof, processes for making the solid forms, and their therapeutic methods of use. Also provided is a process for preparing osanetant, and intermediates for use in the same.

### CROSS REFERENCE TO RELATED APPLICATIONS

This application claims the benefit under 35 U.S.C. § 119 (e) of U.S. Provisional Application No. 63/218,211, filed on Jul. 2, 2021, which is incorporated by reference herein in its entirety.

### BACKGROUND

A compound can exist in one or more crystalline forms. Crystalline forms of a drug substance can have different physical properties, including melting point, solubility, dissolution rate, optical and mechanical properties, vapor pressure, hygroscopicity, particle shape, density, and flowability. These properties can have a direct effect on the ability to process and/or manufacture a compound as a drug product. Crystalline forms can also exhibit different stabilities and bioavailability. To ensure the quality. safety, and efficacy of a drug product, it is important to choose a crystalline form that is stable, is manufactured reproducibly, and has favorable physicochemical properties.

### SUMMARY

The present disclosure relates to solid forms of osanetant having improved physicochemical properties as compared to known forms of osanetant, such as the free base or the HCl salt. As described herein, crystalline salt forms of osanetant could be obtained from certain counterions. Where a crystalline salt form of osanetant could be obtained from a specific counterion, a wide range of improved physicochemical properties was observed, including and not limited to stability and/or absence of conversion to other solid forms.

Crystalline salt forms of osanetant descried herein include osanetant methanesulfonic acid Form 1, osanetant methanesulfonic acid Form 2, osanetant sulfuric acid Form 1, osanetant sulfuric acid Form 2, osanetant ethanesulfonic acid Form 1, osanetant ethanesulfonic acid Form 2, osanetant ethane-1,2-disulfonic acid Form 1, osanetant p-toluenesulfonic acid Form 1, osanetant p-toluenesulfonic acid Form 2, osanetant p-toluenesulfonic acid Form 3, osanetant p-toluenesulfonic acid Form 4, osanetant p-toluenesulfonic acid Form 6, osanetant naphthalene-2-sulfonic acid Form 1, osanetant naphthalene-2-sulfonic acid Form 2, osanetant naphthalene-2-sulfonic acid Form 3, osanetant naphthalene-2-sulfonic acid Form 4, osanetant benzenesulfonic acid Form 1, osanetant benzenesulfonic acid Form 2, osanetant benzenesulfonic acid Form 3, osanetant benzenesulfonic acid Form 4, osanetant phosphoric acid Form 1. osanetant phosphoric acid Form 2, and osanetant phosphoric acid Form 3.

Certain crystalline salt forms of osanetant were shown to exhibit enhanced stability. In one embodiment, the present disclosure provides a crystalline salt form of osanetant which is stable after drying (e.g., at 40° C./75% RH). Such crystalline salt forms include osanetant naphthalene-2-sulfonic acid Form 1, osanetant naphthalene-2-sulfonic acid Form 2, osanetant naphthalene-2-sulfonic acid Form 3, osanetant naphthalene-2-sulfonic acid Form 4, osanetant benzenesulfonic acid Form 1, osanetant benzenesulfonic acid Form 3, osanetant p-toluenesulfonic Form 1, osanetant phosphoric acid Form 3, osanetant phosphoric acid Form 4, osanetant benzoic acid Form 1, and osanetant free base Form 2.

It has been further found that osanetant naphthalene-2-sulfonic acid Form 3 and osanetant benzenesulfonic acid Form 3, which crystalline salt forms are not solvated, exhibited enhanced thermal profiles, and osanetant benzenesulfonic acid Form 1, and osanetant p-toluenesulfonic acid Form 1 exhibited enhanced stability (e.g., at 40° C./75% RH).

As such, the present disclosure provides means for providing a superior drug product for the treatment of the diseases and disorders affected by administration of osanetant.

Also disclosed are processes for providing the crystalline salt forms of osanetant, compositions comprising the same, as well as methods of use thereof. In certain embodiments, provided is a composition comprising osanetant, wherein at least about 85% of the osanetant present in the composition is a single crystalline salt form as disclosed herein.

### BRIEF DESCRIPTION OF THE DRAWINGS

FIG. 1 is an X-ray powder diffraction pattern of osanetant methanesulfonic acid Form 1.

FIG. 2 is an X-ray powder diffraction pattern of osanetant methanesulfonic acid Form 2.

FIG. 3 is an X-ray powder diffraction pattern of osanetant sulfuric acid Form 1.

FIG. 4 is an X-ray powder diffraction pattern of osanetant sulfuric acid Form 2.

FIG. 5 is an X-ray powder diffraction pattern of osanetant ethanesulfonic acid Form 1.

FIG. 6 is an X-ray powder diffraction pattern of osanetant ethanesulfonic acid Form 2.

FIG. 7 is an X-ray powder diffraction pattern of osanetant p-toluenesulfonic acid Form 1.

FIG. 8 is an X-ray powder diffraction pattern of osanetant p-toluenesulfonic acid Form 2.

FIG. 9 is an X-ray powder diffraction pattern of osanetant p-toluenesulfonic acid Form 3.

FIG. 10 is an X-ray powder diffraction pattern of osanetant p-toluenesulfonic acid Form 4.

FIG. 11 is an X-ray powder diffraction pattern of osanetant naphthalene-2-sulfonic acid Form 1.

FIG. 12 is an X-ray powder diffraction pattern of osanetant naphthalene-2-sulfonic acid Form 2.

FIG. 13 is an X-ray powder diffraction pattern of osanetant naphthalene-2-sulfonic acid Form 3.

FIG. 14 is an X-ray powder diffraction pattern of osanetant naphthalene-2-sulfonic acid Form 4.

FIG. 15 is an X-ray powder diffraction pattern of osanetant benzenesulfonic acid Form 1.

FIG. 16 is an X-ray powder diffraction pattern of osanetant benzenesulfonic acid Form 2.

FIG. 17 is an X-ray powder diffraction pattern of osanetant benzenesulfonic acid Form 3.

FIG. 18 is an X-ray powder diffraction pattern of osanetant benzenesulfonic acid Form 4.

FIG. 19 is an X-ray powder diffraction pattern of osanetant phosphoric acid Form 1.

FIG. 20 is an X-ray powder diffraction pattern of osanetant phosphoric acid Form 2.

FIG. 21 is an X-ray powder diffraction pattern of osanetant phosphoric acid Form 3.

FIG. 22 is an X-ray powder diffraction pattern of osanetant phosphoric acid Form 4.

FIG. 23 is an X-ray powder diffraction pattern of osanetant L-malic acid Form 1.

FIG. 24 is an X-ray powder diffraction pattern of osanetant L-malic acid Form 2.

FIG. 25 is an X-ray powder diffraction pattern of osanetant benzoic acid Form 1.

FIG. 26 is an X-ray powder diffraction pattern of osanetant acetic acid Form 1.

FIG. 27 is an X-ray powder diffraction pattern of osanetant free base Form 2.

FIG. 28 is simultaneous TG/DSC of osanetant p-toluenesulfonic acid Form 1.

FIG. 29 is simultaneous TG/DSC of osanetant p-toluenesulfonic acid Form 4.

FIG. 30 is a simultaneous TG/DSC of osanetant naphthalene-2-sulfonic acid Form 1.

FIG. 31 is a simultaneous TG/DSC of osanetant naphthalene-2-sulfonic acid Form 2.

FIG. 32 is a simultaneous TG/DSC of osanetant naphthalene-2-sulfonic acid Form 3.

FIG. 33 is a simultaneous TG/DSC of osanetant naphthalene-2-sulfonic acid Form 4.

FIG. 34 is a simultaneous TG/DSC of osanetant benzenesulfonic acid Form 3.

FIG. 35 is a simultaneous TG/DSC of osanetant phosphoric acid Form 3.

FIG. 36 is a simultaneous TG/DSC of osanetant benzoic acid Form 1.

FIG. 37 is a simultaneous TG/DSC of osanetant free base Form 2.

FIG. 38 is an X-ray powder diffraction pattern of osanetant free base and amorphous lyophilized osanetant.

FIG. 39 is an X-ray powder diffraction pattern of osanetant p-toluene sulfonic acid Form 6.

FIG. 40 is a simultaneous TG/DSC of osanetant p-toluene sulfonic acid Form 6.

FIG. 41 is a simultaneous TG/DSC of osanetant benzeneulfonic acid Form 1.

FIG. 42 is an X-ray powder diffraction pattern of osanetant phosphoric acid Form 7.

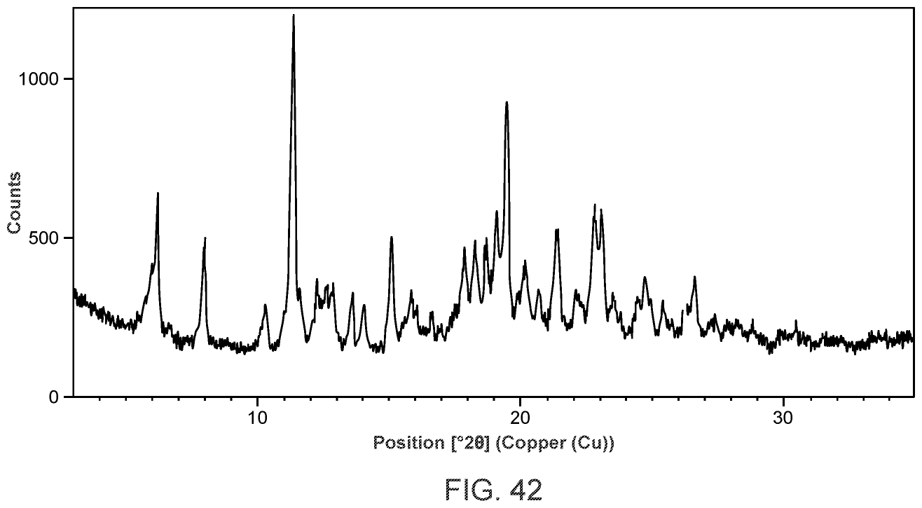

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

Unless defined otherwise, all technical and scientific terms used herein have the same meaning as is commonly understood by one of ordinary skill in the art. As used herein, the below terms have the following meanings unless specified otherwise. Any methods, devices and materials similar or equivalent to those described herein can be used in the practice of the compositions and methods described herein. The following definitions are provided to facilitate understanding of certain terms used frequently herein and are not meant to limit the scope of the present disclosure. All references referred to herein are incorporated by reference in their entirety.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, references to “the agent” includes a plurality of such agents.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X.”

Solid forms of osanetant, including salts or solvates thereof, are provided herein. In certain embodiments, the solid form may be a mixture comprising amorphous osanetant as well as one or more crystalline forms, or crystalline salt forms. In some embodiments, reference to a composition comprising a crystalline salt form or a solvate thereof means that at least 50% to 99% (e.g., at least 50%, at least 55%, at least 60%, at least 65%, at least 70%, at least 75%, at least 80%, at least 85%, at least 90%, at least 95%, or at least 99%) or alternatively, from 50% to 100%, 55% to 100%, 60% to 100%, 65% to 100%, 70 to 100%, 75% to 100%, 80% to 100%, 85% to 100%, 90% to 100%, 95% to 100%, 50% to 99.9%, 55% to 99.9%, 60% to 99.9%, 65% to 99.9%, 70 to 99.9%, 75% to 99.9%, 80% to 99.9%, 85% to 99.9%, 90% to 99.9%, 95 to 99.9%, 50% to 99%, 55% to 99%, 60% to 99%, 65% to 99%, 70 to 99%, 75% to 99%, 80% to 99%, 85% to 99%, 90% to 99%, 95 to 99%, 50% to 95%, 55% to 95%, 60% to 95%, 65% to 95%, 70 to 95%, 75% to 95%, 80% to 95%, 85% to 95%, 90% to 95%, 50% to 90%, 55% to 90%, 60% to 90%, 65% to 90%, 70 to 90%, 75% to 90%, 80% to 90%, 85% to 90%, 50% to 85%, 55% to 85%, 60% to 85%, 65% to 85%, 70 to 85%, 75% to 85%, 80% to 85%, 50% to 80%, 55% to 80%, 60% to 80%, 65% to 80%, 70 to 80%, 75% to 80%, 50% to 75%, 55% to 75%, 60% to 75%, 65% to 75%, or 70 to 75% of the osanetant or a salt or a solvate thereof present in a composition is in the designated form.

The term “solid form” refers to a type of solid-state material that may include amorphous as well as one or more crystalline forms. The terms “crystalline” or “crystalline form” refer to polymorphs, which can include solvates, hydrates, salts, etc. The term “crystalline salt form” refers to polymorphs of an osanetant salt, which can include solvates, hydrates, etc. The term “polymorph” refers to a particular crystal structure having particular physical properties such as X-ray diffraction, melting point, and the like.

As used herein, the term “hydrate” refers to a complex formed by combining a compound and water (i.e., a solvate when the solvent is water).

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

As used herein, the term “administration” refers to introducing an agent into a patient. For example, a therapeutic amount can be administered to the patient, which can be determined by the treating physician, medical professional, or the like. In some embodiments, a therapeutic amount is administered orally. In some embodiments, a therapeutic amount is administered intranasally. In some embodiments, a therapeutic amount is administered subcutaneously. In some embodiments, a therapeutic amount is administered transdermally. In some embodiments, a therapeutic amount is administered intravenously. In some embodiments, a therapeutic amount is administered buccally. The related terms and phrases “administering” and “administration of,” when used in connection with a compound or tablet (and grammatical equivalents) refer both to direct administration, which may be administration to a patient by a medical professional or by self-administration by the patient, and/or to indirect administration, which may be the act of prescribing a drug. Administration entails delivery to the patient of the drug.

The term “dose” or “dosage” refers to the total amount of an active agent (e.g., osanetant or a pharmaceutically acceptable salt thereof) administered to a patient in a single day (24-hour period). The desired dose can be administered once daily. In some embodiments, the desired dose may be administered in one, two, three, four or more sub-doses at appropriate intervals throughout the day, where the cumulative amount of the sub-doses equals the amount of the desired dose administered in a single day. The terms “dose” and “dosage” are used interchangeably herein.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a patient (including a human) who is at risk or has a family history of the disease or condition.

As used herein, the term “patient” or “subject” refers to an animal, such as a mammal (including a human), that has been or will be the object of treatment, observation or experiment. The methods described herein may be useful in human therapy and/or veterinary applications. In some embodiments, the patient is a mammal. In one embodiment, the patient is a human. The patient can be a male or a female.

The term “therapeutically effective amount” or “effective amount” refers to the amount of osanetant administered sufficient to effect treatment when administered to a subject, will have the intended therapeutic effect, e.g., alleviation, amelioration, palliation or elimination of one or more manifestations of the condition in the patient. The amount of osanetant administered refers to the amount of osanetant present, which when referred to by mass, does not include the counterion. For example, if the effective amount of a crystalline salt form is determined to be 25 mg/day, the patient would be administered a sufficient amount of the crystalline salt form such that the dose of osanetant administered is 25 mg/day. The therapeutically effective amount may vary depending on the subject, and disease or condition being treated, the weight and age of the subject, the severity of the disease or condition, and the manner of administering, which can readily be determined by one or ordinary skill in the art.

The full therapeutic effect does not necessarily occur by administration of one dose, and can occur only after administration of a series of doses and can be administered in one dose form or multiples thereof. For example, 50 mg of the osanetant can be administered in a single 50 mg strength tablet or two 25 mg strength tablets. Thus, a therapeutically effective amount may be administered in one or more administrations.

In some embodiments, the phrase “substantially shown in FIG.” or “substantially shown in Figure” as applied to an X-ray powder diffractogram is meant to include a variation of ±0.2°2θ or ±0.1° 2θ, as applied to DSC thermograms is meant to include a variation of ±3° Celsius, and as applied to thermogravimetric analysis (TGA) is meant to include a variation of ±2% in weight loss.

Crystalline Salt Forms

Provided herein are solid forms, including crystalline salt forms, of (R)-N-(1-(3-(1-benzoyl-3-(3,4-dichlorophenyl)piperidin-3-yl)propyl)-4-phenylpiperidin-4-yl)-N-methylacetamide (osanetant), processes of preparing the forms, as well as compositions and methods of use thereof.

The chemical name of osanetant is (R)-N-(1-(3-(1-benzoyl-3-(3,4-dichlorophenyl)piperidin-3-yl)propyl)-4-phenylpiperidin-4-yl)-N-methylacetamide, and has the following structure:

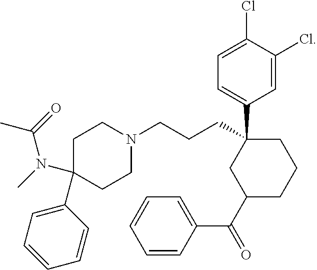 [SMILES: CC(=O)N(C)C1(c2ccccc2)CCN(CCC[C@]2(c3ccc(Cl)c(Cl)c3)CCCC(C(=O)c3ccccc3)C2)CC1]

Osanetant was originally developed for the treatment of schizophrenia and other central nervous system disorders. Osanetant for use in providing the crystalline salt forms described herein can be purchased from commercial sources or can synthesized using the method described herein or published procedures.

In certain embodiments, provided is a solid form of an osanetant salt selected from the group consisting of osanetant methanesulfonic acid, osanetant sulfuric acid, osanetant ethanesulfonic acid, osanetant p-toluenesulfonic acid, osanetant naphthalene-2-sulfonic acid, osanetant benzenesulfonic acid, osanetant phosphoric acid, osanetant L-malic acid, osanetant benzoic acid, and osanetant acetic acid. In certain embodiments, the solid form is a crystalline salt form.

In certain embodiments, the crystalline salt form of (R)-N-(1-(3-(1-benzoyl-3-(3,4 -dichlorophenyl)piperidin-3-yl)propyl)-4-phenylpiperidin-4-yl)-N-methylacetamide (osanetant) is a crystalline form of an osanetant salt selected from the group consisting of osanetant methanesulfonic acid, osanetant sulfuric acid, osanetant ethanesulfonic acid, osanetant p-toluenesulfonic acid, osanetant naphthalene-2-sulfonic acid, osanetant benzenesulfonic acid, osanetant phosphoric acid, osanetant L-malic acid, osanetant benzoic acid, and osanetant acetic acid.

In certain embodiments, the crystalline salt form is selected from the group consisting of osanetant methanesulfonic acid Form 1, osanetant methanesulfonic acid Form 2, osanetant sulfuric acid Form 1, osanetant sulfuric acid Form 2, osanetant ethanesulfonic acid Form 1, osanetant ethanesulfonic acid Form 2, osanetant p-toluenesulfonic acid Form 1, osanetant p-toluenesulfonic acid Form 2, osanetant p-toluenesulfonic acid Form 3, osanetant p-toluenesulfonic acid Form 4, osanetant p-toluenesulfonic acid Form 6, osanetant naphthalene-2-sulfonic acid Form 1, osanetant naphthalene-2-sulfonic acid Form 2. osanetant naphthalene-2-sulfonic acid Form 3, osanetant naphthalene-2-sulfonic acid Form 4, osanetant benzenesulfonic acid Form 1, osanetant benzenesulfonic acid Form 2, osanetant benzenesulfonic acid Form 3, osanetant benzenesulfonic acid Form 4, osanetant phosphoric acid Form 1, osanetant phosphoric acid Form 2, osanetant phosphoric acid Form 3, osanetant phosphoric acid Form 4, osanetant L-malic acid Form 1, osanetant L-malic acid Form 2, osanetant benzoic acid Form 1, osanetant acetic acid Form 1, and osanetant free base Form 2.

In certain embodiments, the crystalline salt form is selected from the group consisting of osanetant p-toluenesulfonic acid Form 1, osanetant p-toluenesulfonic acid Form 6, osanetant benzenesulfonic acid Form 1, and osanetant benzenesulfonic acid Form 3.

In certain embodiments, the crystalline salt form of osanetant is other than osanetant benzenesulfonic acid. In certain embodiments, the crystalline salt form of osanetant is other than osanetant benzenesulfonic acid Form 3. In certain embodiments, the crystalline salt form of osanetant is other than osanetant benzenesulfonic acid Form 1.

In one embodiment, provided is a solid form of (R)-N-(1-(3-(1-benzoyl-3-(3,4 -dichlorophenyl)piperidin-3-yl)propyl)-4-phenylpiperidin-4-yl)-N-methylacetamide methanesulfonic acid (osanetant methanesulfonic acid). In one embodiment, provided is crystalline (R)-N-(1-(3-(1-benzoyl-3 -(3,4-dichlorophenyl)piperidin-3-yl)propyl)-4-phenylpiperidin-4-yl)-N-methylacetamide methanesulfonic acid (osanetant methanesulfonic acid).

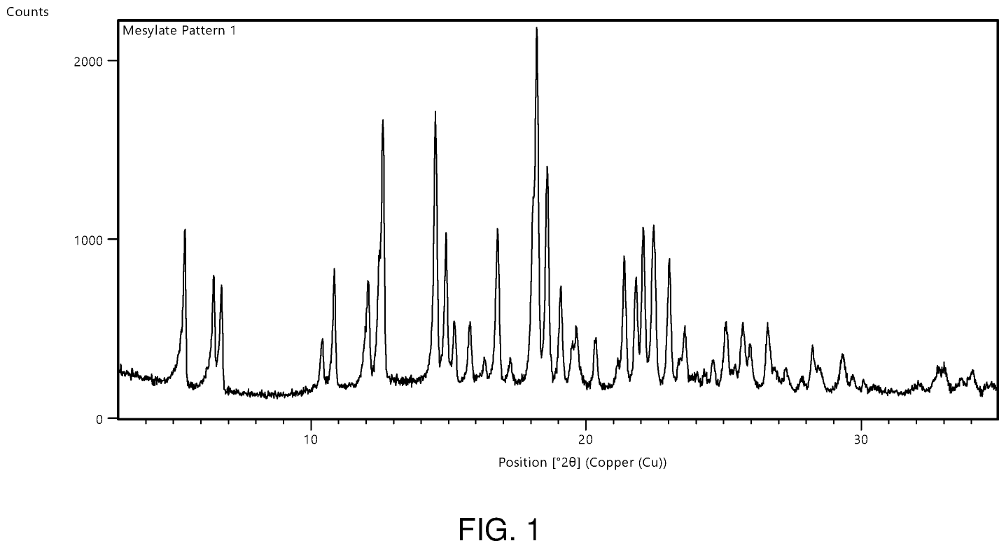

In one embodiment, provided is crystalline (R)-N-(1-(3-(1-benzoyl-3-(3,4 -dichlorophenyl)piperidin-3-yl)propyl)-4-phenylpiperidin-4-yl)-N-methylacetamide methanesulfonic acid (osanetant methanesulfonic acid Form 1) characterized by an X-ray powder diffractogram comprising the following peaks: 18.2±0.2°2θ, 14.5°2θ°2θ±0.2°2θ, and 12.6°2θ±0.2°2θ. In certain embodiments, the diffractogram further comprises one or more additional peaks selected from 16.8°2θ±0.2°2θ, 18.0° 2θ±0.2°2θ, 18.6°2θ±0.2°2θ, and 22.5°2θ±0.2°2θ. In certain embodiments, the diffractogram is substantially as shown in FIG. 1.

In certain embodiments, osanetant methanesulfonic acid Form 1 is characterized by a differential scanning calorimetry (DSC) curve that comprises an endothermic event with a peak at about 153.3° C.

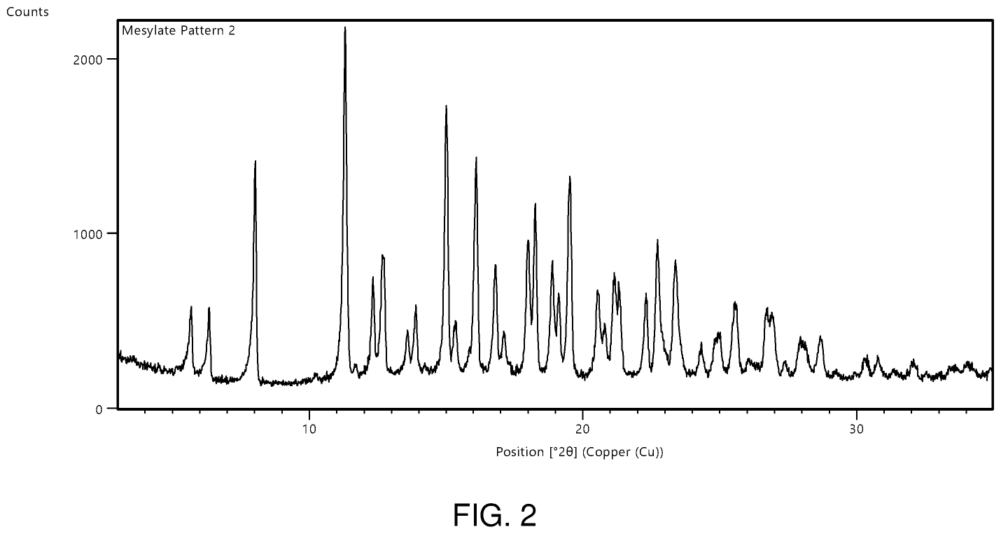

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide methanesulfonic acid (osanetant methanesulfonic acid Form 2) characterized by an X-ray powder diffractogram comprising the following peaks: 11.3°2θ±0.2°2θ, 15.0°2θ±0.2°2θ, and 16.1°2θ±0.2°2θ. In certain embodiments, the diffractogram further comprises one or more additional peaks selected from 8.0°2θ±0.2°2θ, 18.3°2θ±0.2°2θ, and 19.5°2θ±0.2°2θ. In certain embodiments, the diffractogram is substantially as shown in FIG. 2.

In one embodiment, provided is a solid form of (R)-N-(1-(3-(1-benzoyl-3-(3,4-dichlorophenyl)piperidin-3-yl)propyl)-4-phenylpiperidin-4-yl)-N-methylacetamide sulfuric acid (osanetant sulfuric acid). In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide sulfuric acid (osanetant sulfuric acid).

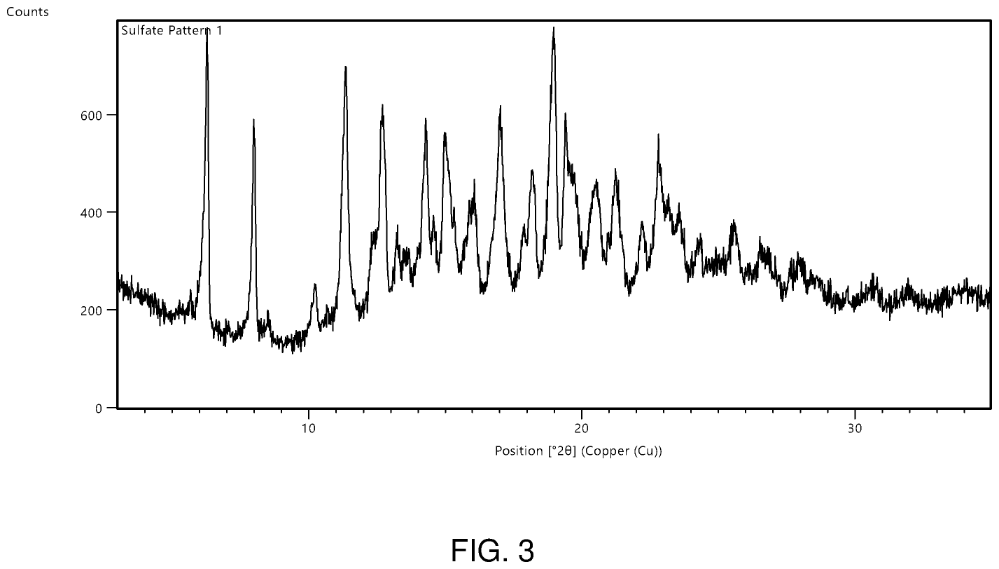

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide sulfuric acid (osanetant sulfuric acid Form 1) characterized by an X-ray powder diffractogram comprising the following peaks: 19.0°2θ±0.2°2θ, 6.3°2θ±0.2°2θ, and 11.4°2θ±0.2°2θ. In certain embodiments, the diffractogram further comprises one or more additional peaks selected from 12.7°2θ±0.2°2θ, 14.3°2θ±0.2°2θ, 17.0°2θ±0.2°2θ, and 19.4°2θ±0.2°2θ. In certain embodiments, the diffractogram is substantially as shown in FIG. 3.

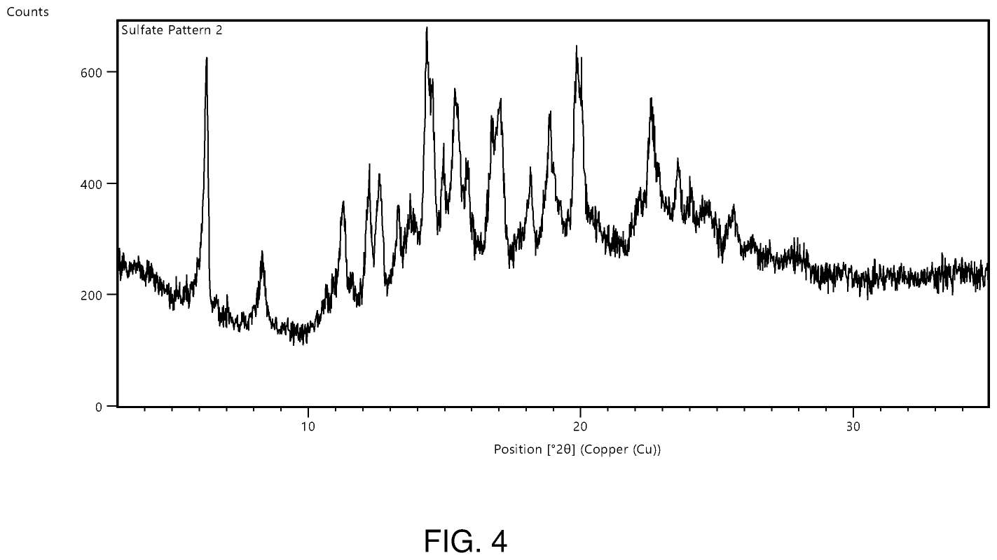

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide sulfuric acid (osanetant sulfuric acid Form 2) characterized by an X-ray powder diffractogram comprising the following peaks: 6.3°2θ±0.2°2θ, 14.3°2θ±0.2°2θ, and 15.4°2θ±0.2°2θ. In certain embodiments, the diffractogram further comprises one or more additional peaks selected from 17.1°2θ±0.2°2θ, 19.8°2θ±0.2°2θ, and 22.6°2θ±0.2°2θ. In certain embodiments, the diffractogram is substantially as shown in FIG. 4.

In one embodiment, provided is a solid form of (R)-N-(1-(3-(1-benzoyl-3-(3,4-dichlorophenyl)piperidin-3-yl)propyl)-4-phenylpiperidin-4-yl)-N-methylacetamide ethanesulfonic acid (osanetant ethanesulfonic acid). In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide ethanesulfonic acid (osanetant ethanesulfonic acid).

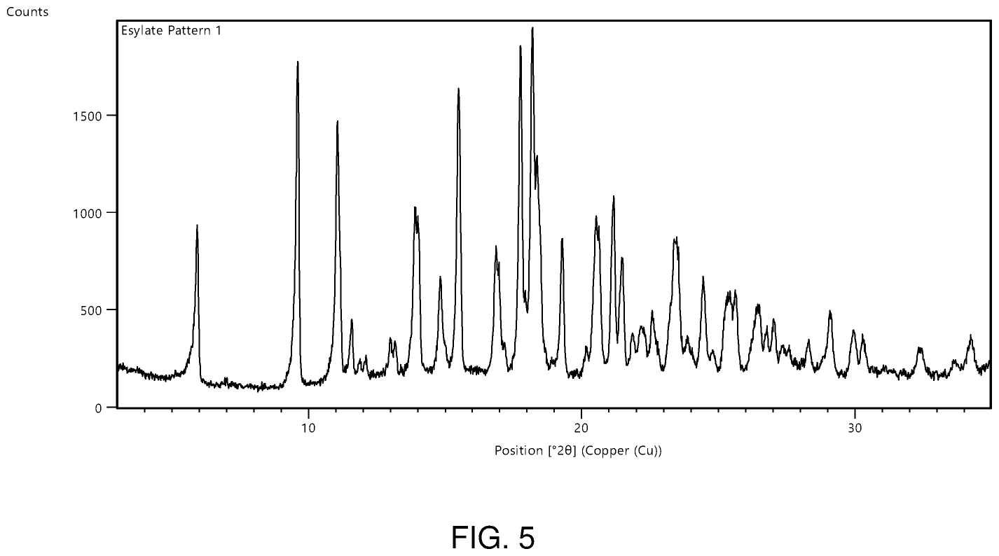

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide ethanesulfonic acid (osanetant ethanesulfonic acid Form 1) characterized by an X-ray powder diffractogram comprising the following peaks: 9.6°2θ±0.2°2θ, 17.8°2θ±0.2°2θ, and 18.2°2θ±0.2°2θ. In certain embodiments, the diffractogram further comprises one or more additional peaks selected from 11.1°2θ±0.2°2θ, 15.5°2θ±0.2°2θ, and 18.4°2θ±0.2°2θ. In certain embodiments, the diffractogram is substantially as shown in FIG. 5.

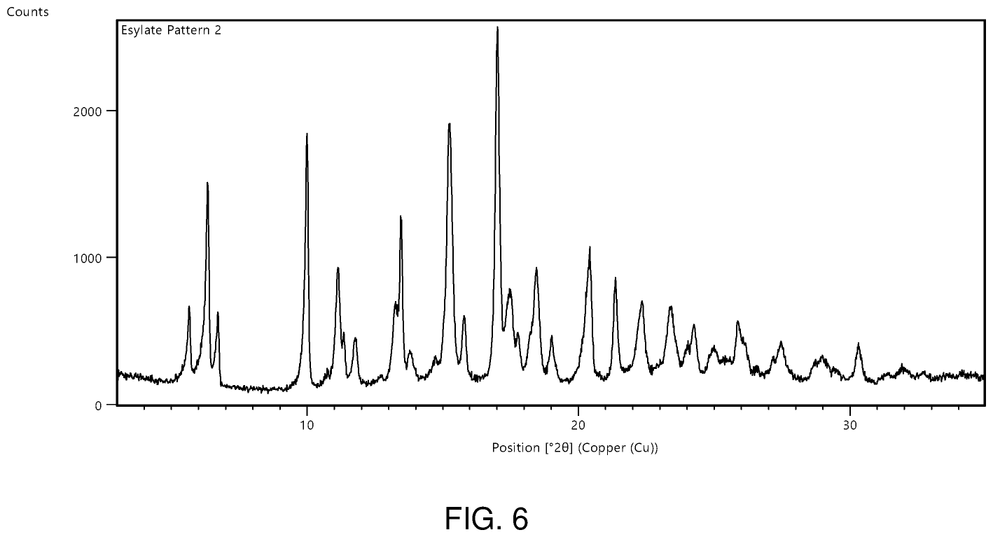

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide ethanesulfonic acid (osanetant ethanesulfonic acid Form 2) characterized by an X-ray powder diffractogram comprising the following peaks: 10.0°2θ±0.2°2θ, 15.2°2θ±0.2°2θ, and 17.0°2θ±0.2°2θ. In certain embodiments, the diffractogram further comprises one or more additional peaks selected from 6.4°2θ±0.2°2θ, 13.5°2θ±0.2°2θ, and 20.4°2θ±0.2°2θ. In certain embodiments, the diffractogram is substantially as shown in FIG. 6.

In one embodiment, provided is a solid form of (R)-N-(1-(3-(1-benzoyl-3-(3,4-dichlorophenyl)piperidin-3-yl)propyl)-4-phenylpiperidin-4-yl)-N-methylacetamide p-toluenesulfonic acid (osanetant p-toluenesulfonic acid). In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide p-toluenesulfonic acid (osanetant p-toluenesulfonic acid). In certain embodiments, osanetant p-toluenesulfonic acid Form 1 may be referred to as osanetant p-toluenesulfonic acid Form 1 monohydrate.

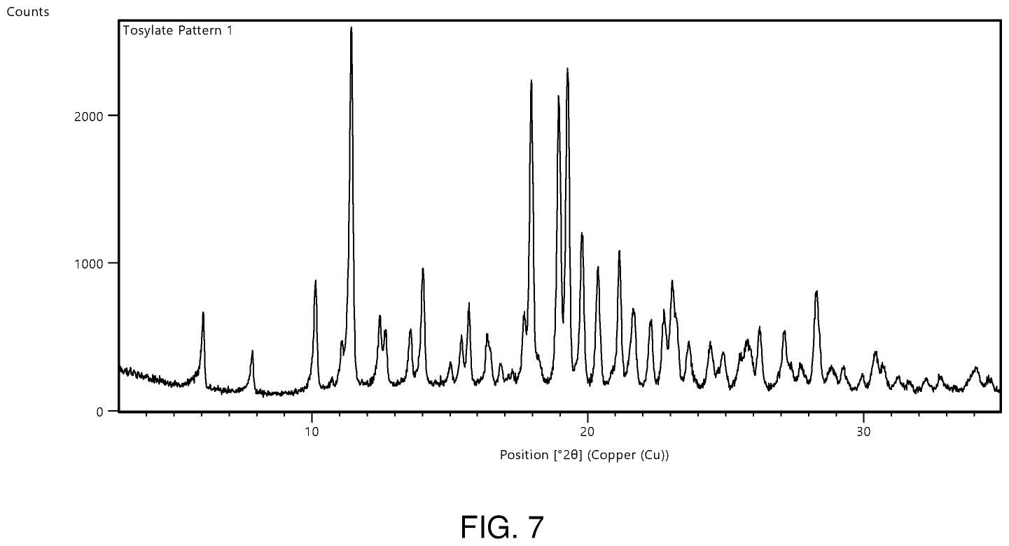

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide p-toluenesulfonic acid (osanetant p-toluenesulfonic acid Form 1, or osanetant tosylate Form 1) characterized by an X-ray powder diffractogram comprising the following peaks: 11.4°2θ±0.2°2θ, 18.0°2θ±0.2°2θ, and 19.3°2θ±0.2°2θ. In certain embodiments, the diffractogram further comprises one or more additional peaks selected from 19.0°2θ±0.2°2θ, 19.8°2θ±0.2°2θ, and 21.1°2θ±0.2°2θ. In certain embodiments, the diffractogram is substantially as shown in FIG. 7.

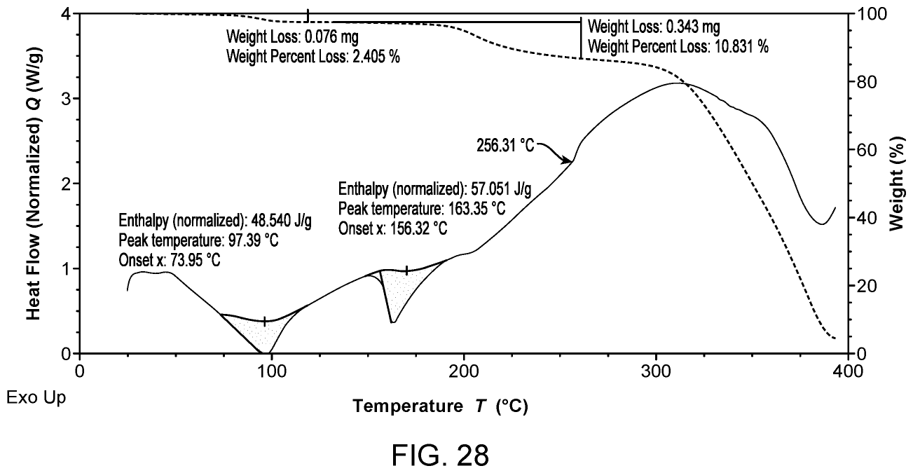

In certain embodiments, osanetant p-toluenesulfonic acid Form 1 is characterized by a differential scanning calorimetry (DSC) curve that comprises a broad endothermic event (peak at about 97.4° C.), and optionally endothermic event onset at about 156.3° C. (peak at about 163.4° C.). In certain embodiments, the DSC curve is substantially as shown in FIG. 28.

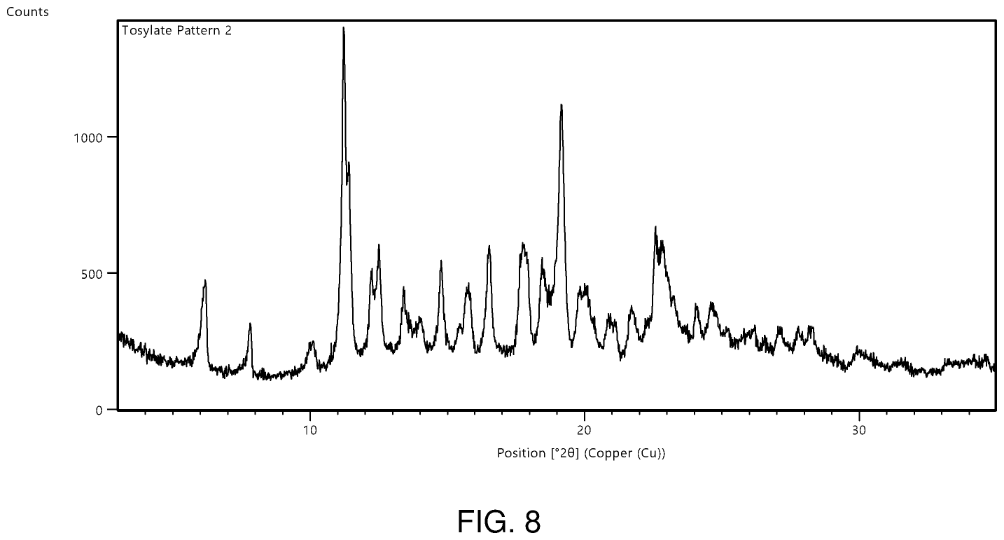

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide p-toluenesulfonic acid (osanetant p-toluenesulfonic acid Form 2, or osanetant tosylate Form 2) characterized by an X-ray powder diffractogram comprising the following peaks: 11.2°2θ±0.2°2θ, 11.4°2θ±0.2°2θ, and 19.2°2θ±0.2°2θ. In certain embodiments, the diffractogram further comprises one or more additional peaks selected from 12.5°2θ±0.2°2θ, 16.5°2θ±0.2°2θ, 22.6°2θ±0.2°2θ, and 22.9°2θ±0.2°2θ. In certain embodiments, the diffractogram is substantially as shown in FIG. 8.

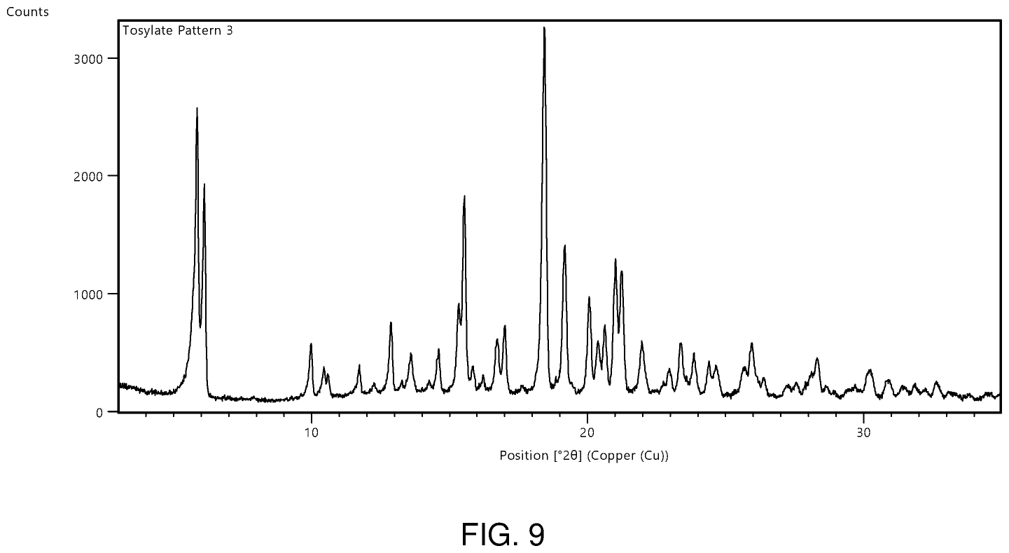

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide p-toluenesulfonic acid (osanetant p-toluenesulfonic acid Form 3, or osanetant tosylate Form 3) characterized by an X-ray powder diffractogram comprising the following peaks: 5.9°2θ±0.2°2θ, 6.1°2θ±0.2°2θ, and 18.4°2θ±0.2°2θ. In certain embodiments, the diffractogram further comprises one or more additional peaks selected from 15.5°2θ±0.2°2θ, 19.2°2θ±0.2°2θ, and 21.0°2θ±0.2°2θ. In certain embodiments, the diffractogram is substantially as shown in FIG. 9.

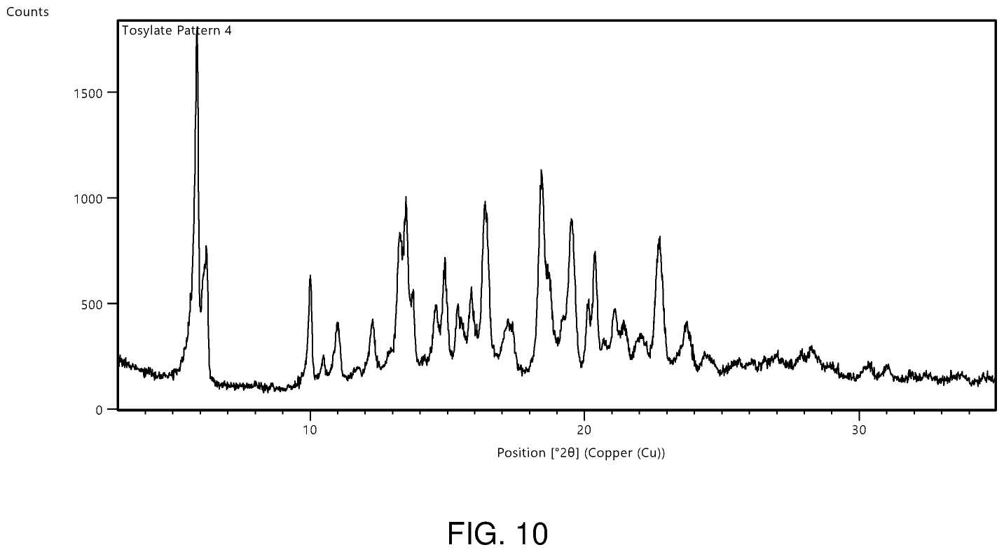

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide p-toluenesulfonic acid (osanetant p-toluenesulfonic acid Form 4, or osanetant tosylate Form 4) characterized by an X-ray powder diffractogram comprising the following peaks: 5.9°2θ±0.2°2θ, 13.5°2θ±0.2°2θ, and 18.4°2θ±0.2°2θ. In certain embodiments, the diffractogram further comprises one or more additional peaks selected from 13.3°274 ±0.2°2θ, 16.3°2θ±0.2°2θ, 19.5°2θ±0.2°20θand 22.7°2θ±0.2°2θ. In certain embodiments, the diffractogram is substantially as shown in FIG. 10.

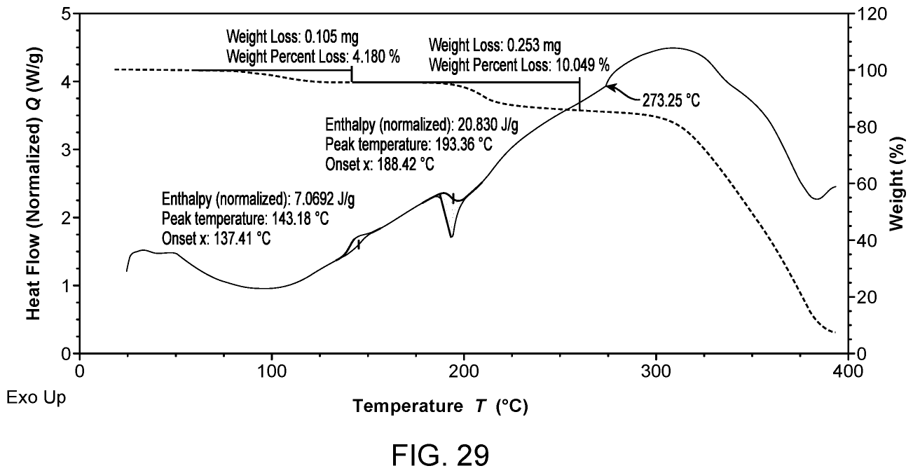

In certain embodiments, osanetant p-toluenesulfonic acid Form 4 is characterized by a differential scanning calorimetry (DSC) curve that comprises a small exothermic event with an onset at about 137.4° C. (peak at about 143.2° C.), and an endothermic event with an onset at about 188.4° C. (peak at 193.4° C.). In certain embodiments, the DSC curve is substantially as shown in FIG. 29.

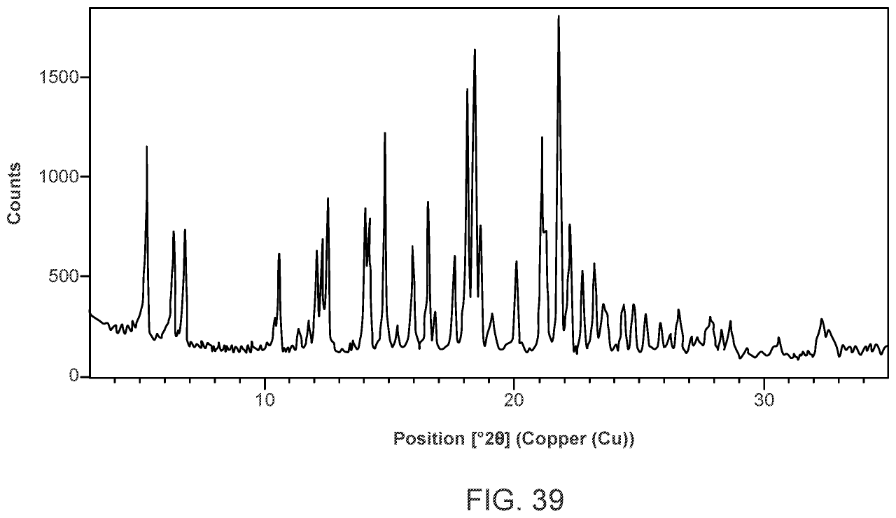

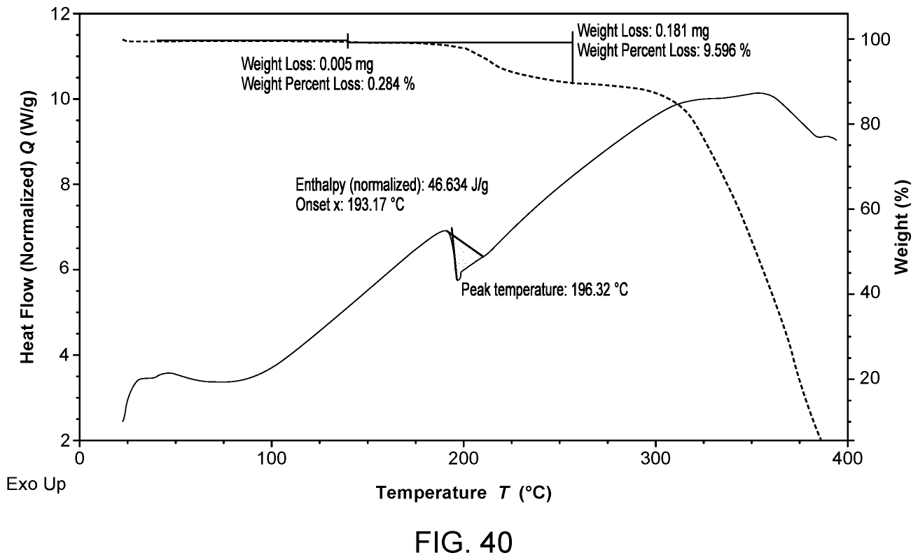

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide p-toluenesulfonic acid (osanetant p-toluenesulfonic acid Form 6, or osanetant tosylate Form 6) characterized by an X-ray powder diffractogram comprising the following peaks: 18.2°2θ±0.2°2θ, 18.4°2θ±0.2°2θ, and 21.8°2θ±0.2°20θIn certain embodiments, the diffractogram further comprises one or more additional peaks selected from 14.9°2θ±0.2°2θ, 16.6°2θ±0.2°2θ, and 21.2°2θ±0.2°2θ. In certain embodiments, the diffractogram is substantially as shown in FIG. 39. In certain embodiments, osanetant p-toluenesulfonic acid Form 6 is characterized by a differential scanning calorimetry (DSC) curve that comprises an endothermic event with an onset at about 193.2° C. (peak at 196.3° C.). In certain embodiments, the DSC curve is substantially as shown in FIG. 40. In certain embodiments, osanetant p-toluenesulfonic acid Form 6 may be referred to as osanetant p-toluenesulfonic acid Form 6 anhydrous.

In certain embodiments, osanetant p-toluenesulfonic acid Form 5, or osanetant tosylate Form 5, is obtained as an unstable anhydrous form, obtained from dehydration of Form 1; however Form 5 rehydrates to Form 1 under ambient conditions.

In one embodiment, provided is a solid form of (R)-N-(1-(3-(1-benzoyl-3-(3,4-dichlorophenyl)piperidin-3-yl)propyl)-4-phenylpiperidin-4-yl)-N-methylacetamide naphthalene-2-sulfonic acid (osanetant naphthalene-2-sulfonic acid). In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide naphthalene-2-sulfonic acid (osanetant naphthalene-2-sulfonic acid).

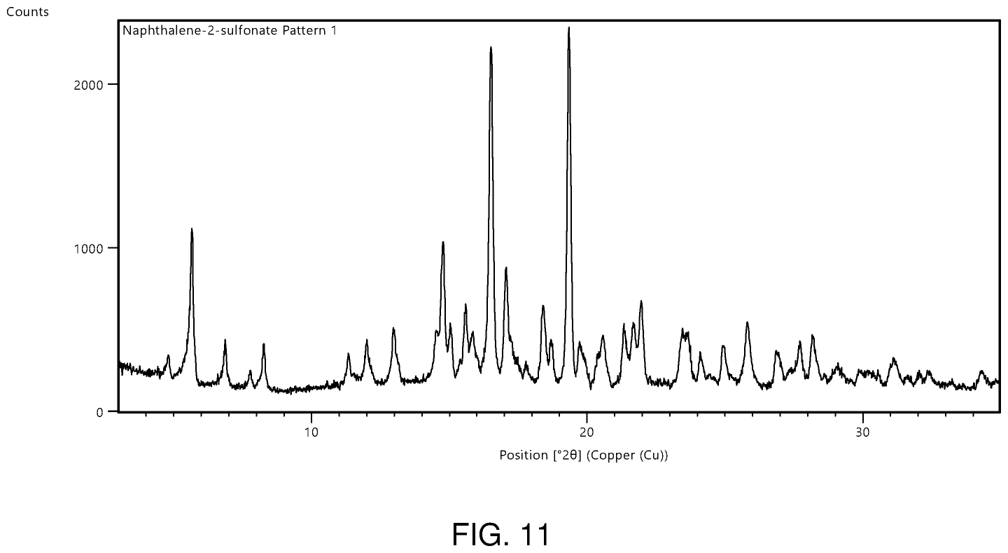

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide naphthalene-2-sulfonic acid (osanetant naphthalene-2-sulfonic acid Form 1) characterized by an X-ray powder diffractogram comprising the following peaks: 5.7°2θ±0.2°2θ, 16.5°2θ±0.2°2θ, and 19.3°2θ±0.2°2θ. In certain embodiments, the diffractogram further comprises one or more additional peaks selected from 14.8°2θ±0.2°2θ, 15.6°2θ±0.2°2θ, 17.0°2θ±0.2°2θ, and 22.0°2θ±0.2°2θ. In certain embodiments, the diffractogram is substantially as shown in FIG. 11.

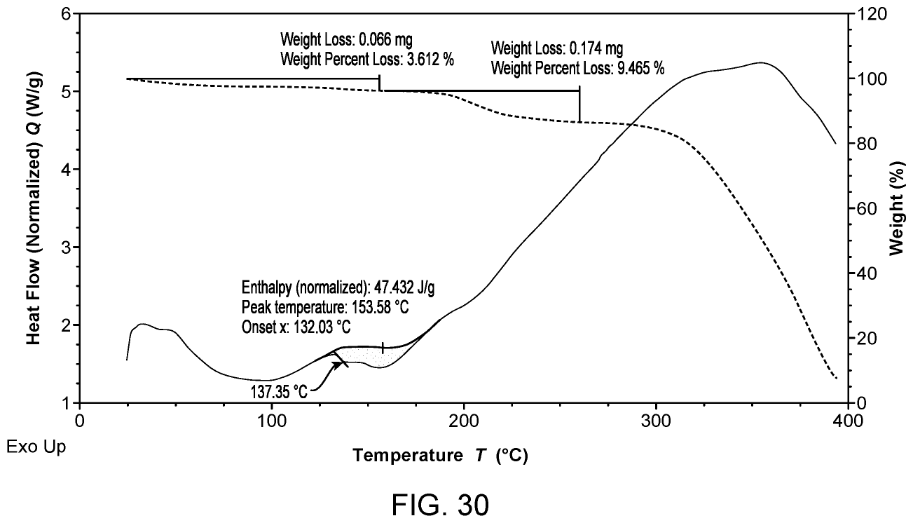

In certain embodiments, osanetant naphthalene-2-sulfonic acid Form 1 is characterized by a differential scanning calorimetry (DSC) curve that comprises overlapping endothermic events with peaks at about 137.4° C. and about 153.6° C. In certain embodiments, the DSC curve is substantially as shown in FIG. 30.

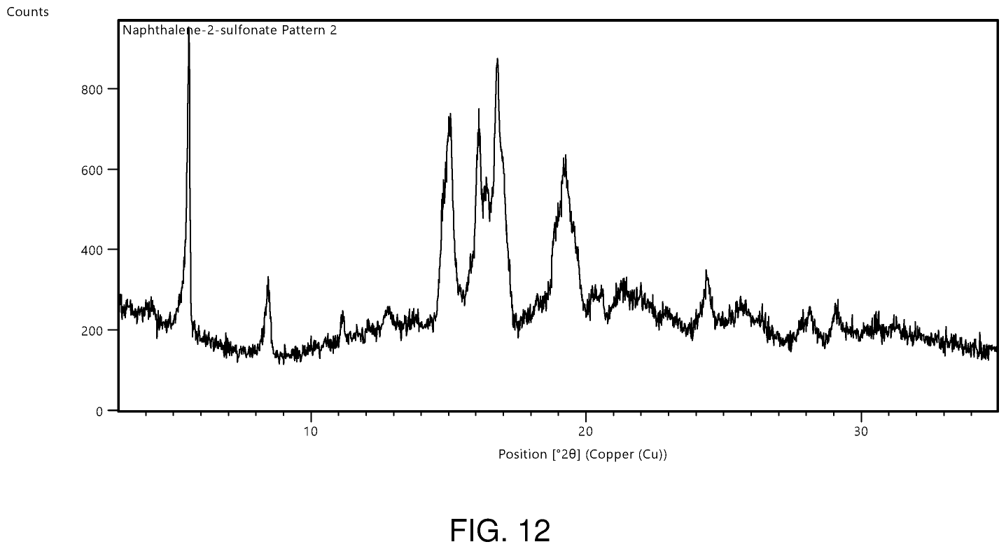

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide naphthalene-2-sulfonic acid (osanetant naphthalene-2-sulfonic acid Form 2) characterized by an X-ray powder diffractogram comprising the following peaks: 5.6°2θ±0.2°2θ, 15.1°2θ±0.2°2θ, and 16.8°2θ±0.2°2θ. In certain embodiments, the diffractogram further comprises one or more additional peaks selected from 16.1°2θ±0.2°2θ, 18.9°2θ±0.2°2θ, and 19.2°2θ±0.2°2θ. In certain embodiments, the diffractogram is substantially as shown in FIG. 12.

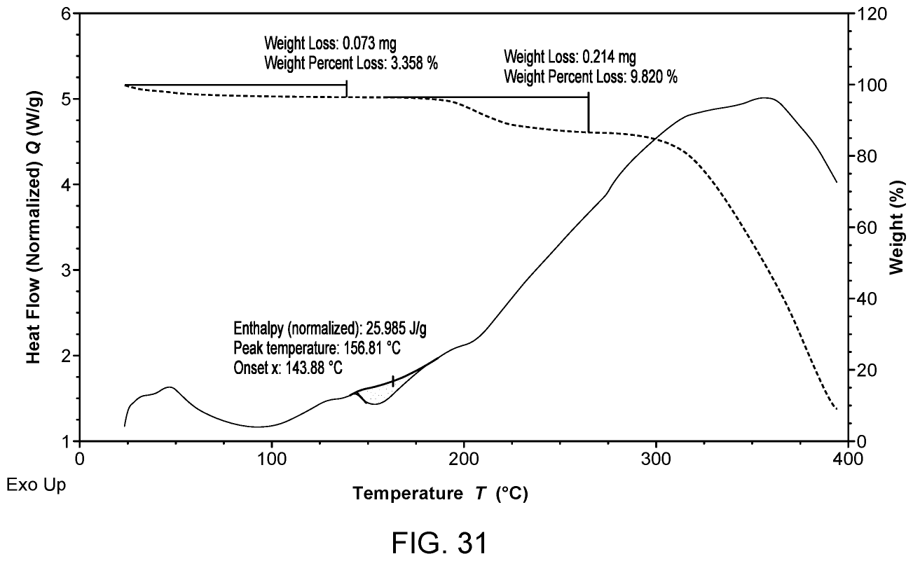

In certain embodiments, osanetant naphthalene-2-sulfonic acid Form 2 is characterized by a differential scanning calorimetry (DSC) curve that comprises an endothermic event with an onset at about 143.9° C. (peak at about 156.8° C.). In certain embodiments, the DSC curve is substantially as shown in FIG. 31.

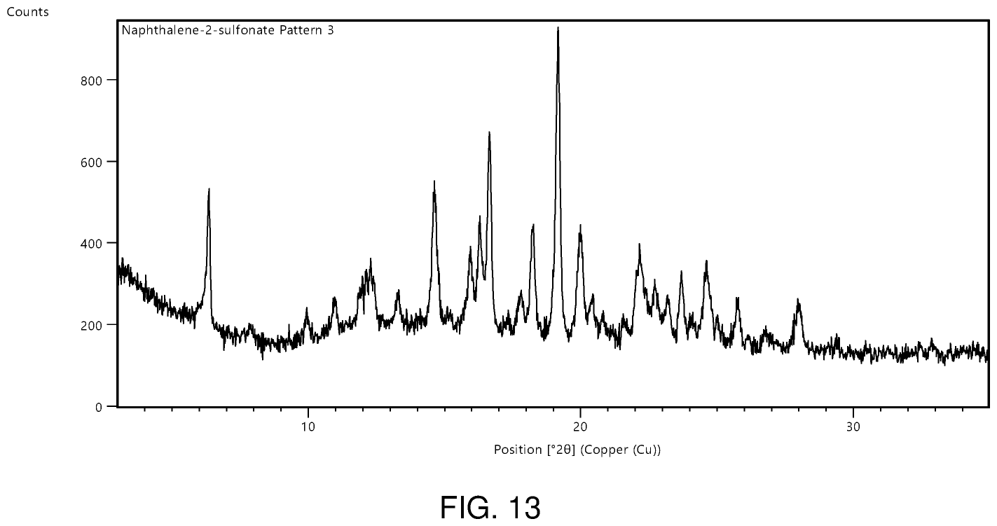

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide naphthalene-2-sulfonic acid (osanetant naphthalene-2-sulfonic acid Form 3) characterized by an X-ray powder diffractogram comprising the following peaks: 14.6°2θ±0.2°2θ, 16.7°2θ±0.2°2θ, and 19.2°2θ±0.2°2θ. In certain embodiments, the diffractogram further comprises one or more additional peaks selected from 6.4°2θ±0.2°2θ, 16.3°2θ±0.2°2θ, and 18.2°2θ±0.2°2θ. In certain embodiments, the diffractogram is substantially as shown in FIG. 13.

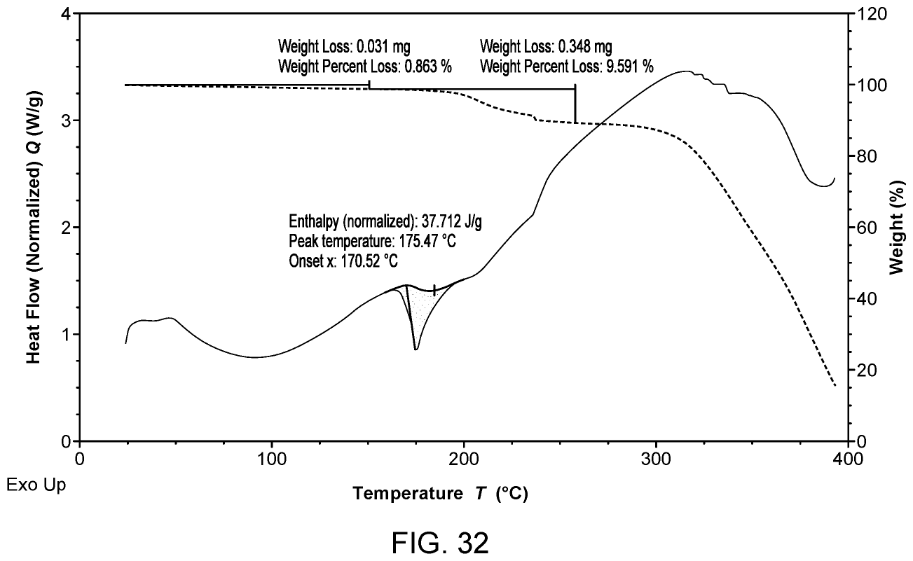

In certain embodiments, osanetant naphthalene-2-sulfonic acid Form 3 is characterized by a differential scanning calorimetry (DSC) curve that comprises an endotherm with an onset at about 170.5° C. (peak at about 175.5° C.). In certain embodiments, the DSC curve is substantially as shown in FIG. 32.

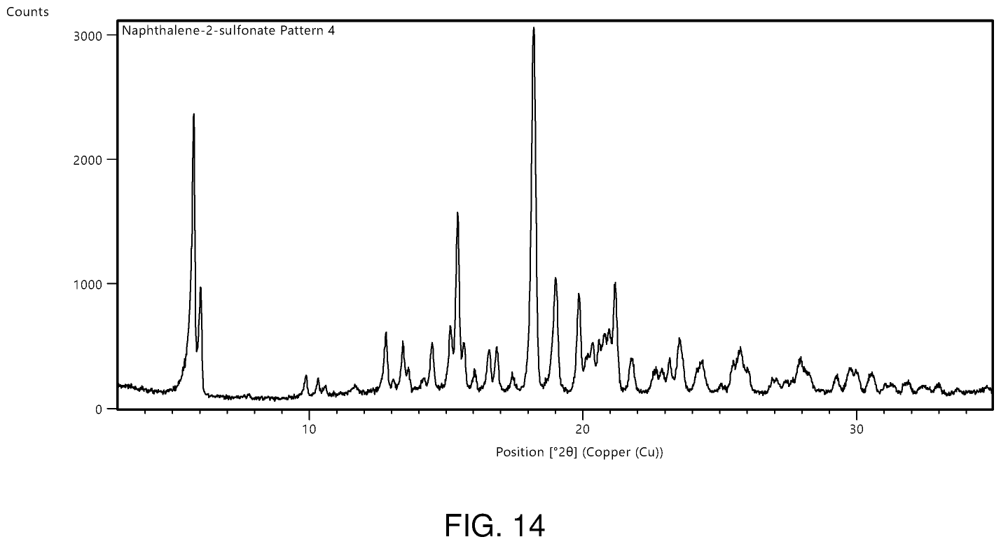

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide naphthalene-2-sulfonic acid (osanetant naphthalene-2-sulfonic acid Form 4) characterized by an X-ray powder diffractogram comprising the following peaks: 5.8°2θ±0.2°2θ, 15.4°2θ±0.2°2θ, and 18.2°2θ±0.2°2θ. In certain embodiments, the diffractogram further comprises one or more additional peaks selected from 6.0°2θ±0.2°2θ, 19.0°2θ±0.2°2θ, 19.9°2θ±0.2°2θ, and 21.2°2θ±0.2°2θ. In certain embodiments, the diffractogram is substantially as shown in FIG. 14.

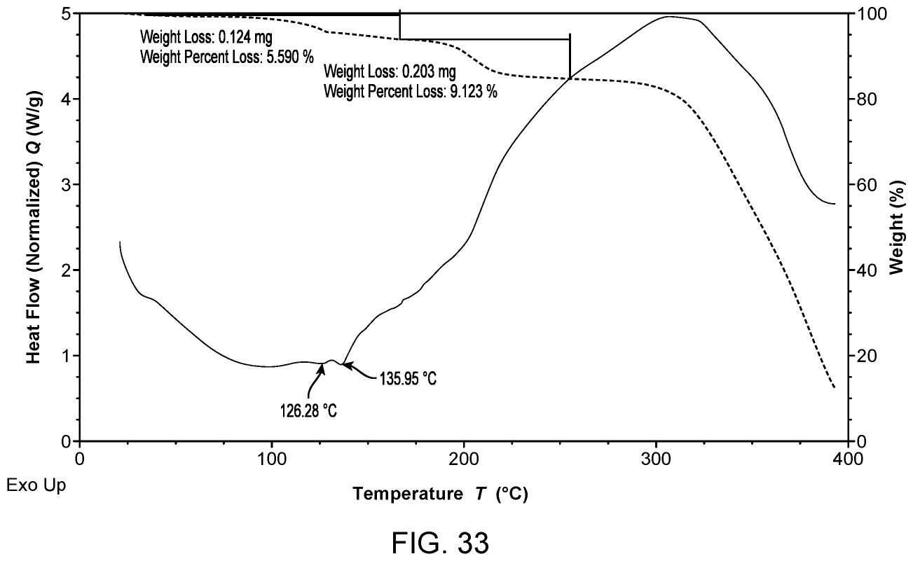

In certain embodiments, osanetant naphthalene-2-sulfonic acid Form 4 is characterized by the DSC curve is substantially as shown in FIG. 33.

In one embodiment, provided is a solid form of (R)-N-(1-(3-(1-benzoyl-3-(3,4-dichlorophenyl)piperidin-3-yl)propyl)-4-phenylpiperidin-4-yl)-N-methylacetamide benzenesulfonic acid (osanetant benzenesulfonic acid). In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide benzenesulfonic acid (osanetant benzenesulfonic acid).

In certain embodiments, the crystalline osanetant benzenesulfonic acid is non-solvated (i.e., is not a solvate or a hydrate). In certain embodiments, the crystalline osanetant benzenesulfonic acid is not a 4-methyl-2-pentanone solvate. In certain embodiments, the crystalline osanetant benzenesulfonic acid is a monohydrate.

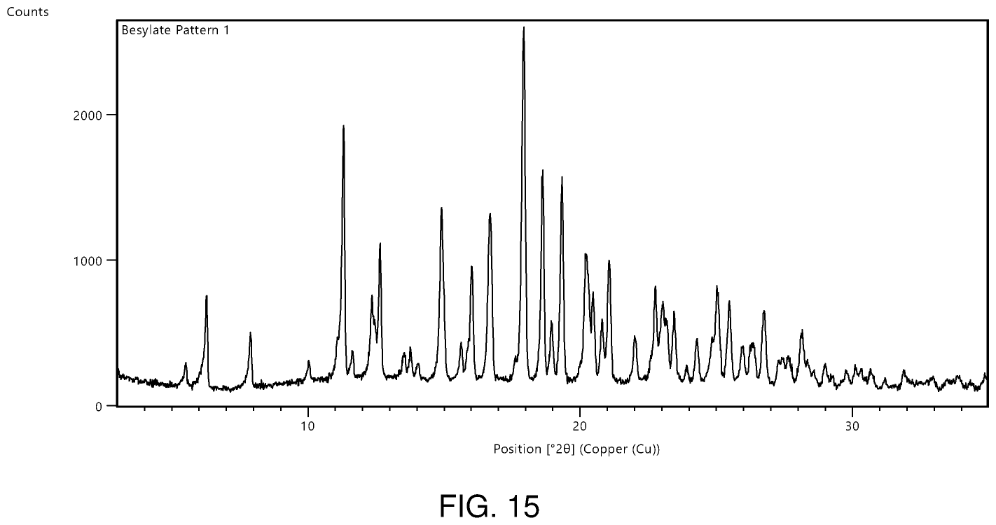

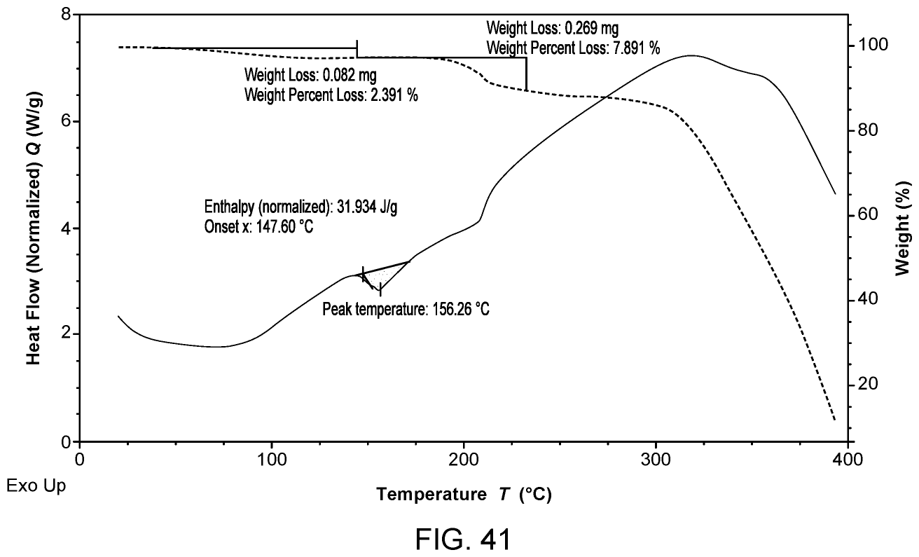

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide benzenesulfonic acid (osanetant benzenesulfonic acid Form 1, or osanetant besylate Form 1) characterized by an X-ray powder diffractogram comprising the following peaks: 11.3°2θ±0.2°2θ, 17.9°2θ±0.2°2θ, and 19.3°2θ±0.2°2θ. In certain embodiments, the diffractogram further comprises one or more additional peaks selected from 14.9°2θ±0.2°2θ, 16.7°2θ±0.2°2θ, and 18.6°2θ±0.2°2θ. In certain embodiments, the diffractogram is substantially as shown in FIG. 15. In certain embodiments, osanetant benzenesulfonic acid Form 1 may be referred to as osanetant benzenesulfonic acid Form 1 monohydrate. In certain embodiments, osanetant benzenesulfonic acid Form 1 is characterized by a differential scanning calorimetry (DSC) curve that comprises an endotherm at with an onset of about 147.6° C. (peak at about 156.3° C.). In certain embodiments, the DSC curve is substantially as shown in FIG. 41.

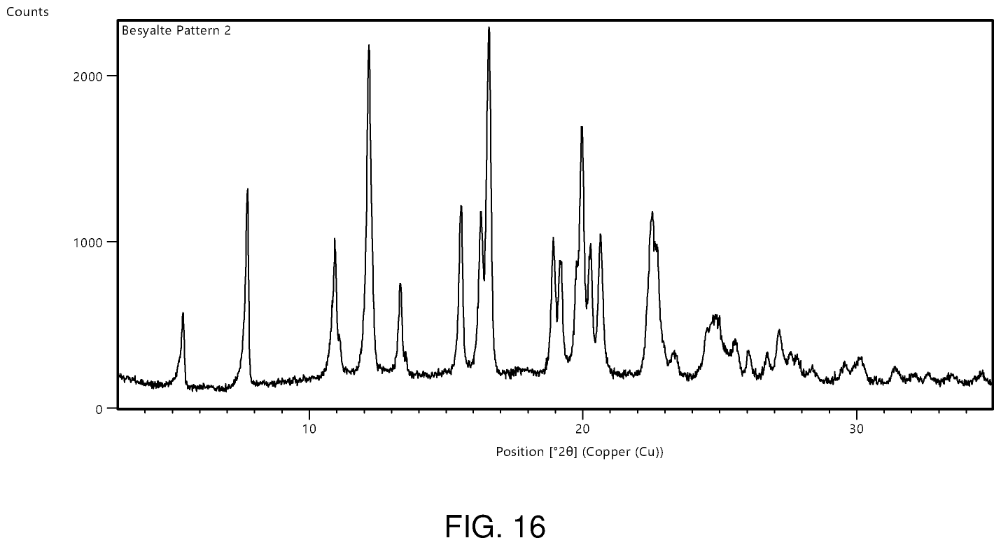

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide benzenesulfonic acid (osanetant benzenesulfonic acid Form 2, or osanetant besylate Form 2) characterized by an X-ray powder diffractogram comprising the following peaks: 12.2°2θ±0.2°2θ, 16.6°2θ±0.2°2θ, and 20.0°2θ±0.2°2θ. In certain embodiments, the diffractogram further comprises one or more additional peaks selected from 7.8°2θ±0.2°2θ. 15.6°2θ±0.2°2θ, 16.3°2θ±0.2°2θ, and 22.5°2θ±0.2°2θ. In certain embodiments, the diffractogram is substantially as shown in FIG. 16.

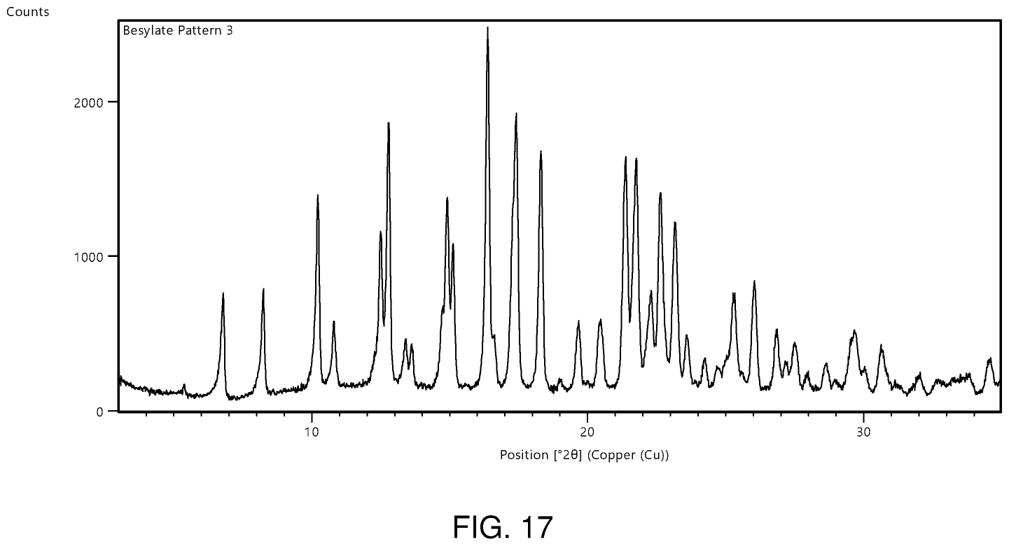

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide benzenesulfonic acid (osanetant benzenesulfonic acid Form 3, or osanetant besylate Form 3) characterized by an X-ray powder diffractogram comprising the following peaks: 12.8°2θ±0.2°2θ, 16.4°2θ±0.2°2θ, and 17.4°2θ±0.2°2θ. In certain embodiments, the diffractogram further comprises one or more additional peaks selected from 18.3°2θ±0.2°2θ, 21.4°2θ±0.2°2θ, and 21.8°2θ±0.2°2θ. In certain embodiments, the diffractogram is substantially as shown in FIG. 17.

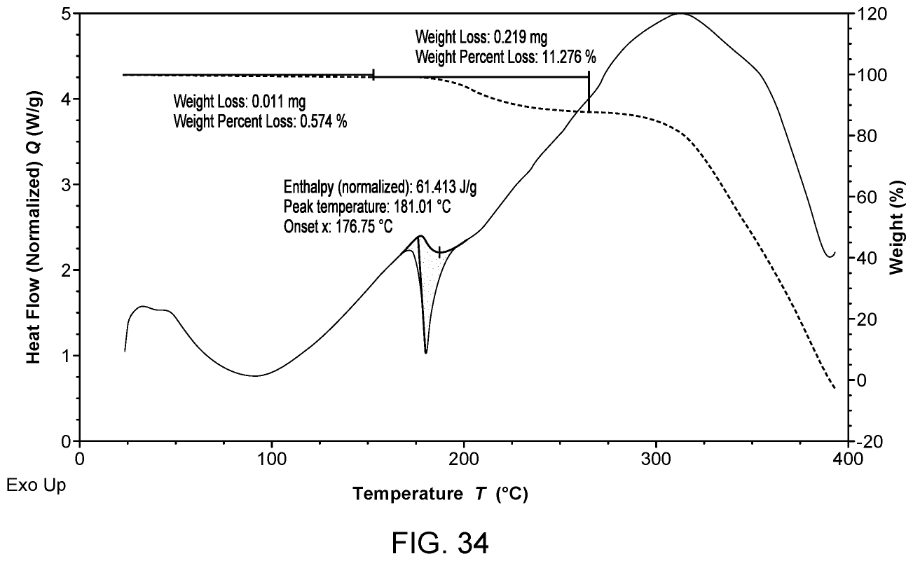

In certain embodiments, osanetant benzenesulfonic acid Form 3 is characterized by a differential scanning calorimetry (DSC) curve that comprises an endotherm at with an onset at about 176.8° C. (peak at about 181.0° C.). In certain embodiments, the DSC curve is substantially as shown in FIG. 34.

In certain embodiments, the osanetant benzenesulfonic acid Form 3 is non-solvated (i.e., is not a solvate or a hydrate). In certain embodiments, the osanetant benzenesulfonic acid Form 3 is not a 4-methyl-2-pentanone solvate. In certain embodiments, the osanetant benzenesulfonic acid Form 3 may be referred to as osanetant benzenesulfonic acid Form 3 anhydrous.

Also provided herein is a process for preparing osanetant benzenesulfonic acid Form 3, which process comprises contacting osanetant free base with benzenesulfonic acid in a suitable solvent, wherein the solvent does not comprise 4-methyl-2-pentanone. Also provided herein is a process for preparing osanetant benzenesulfonic acid Form 3, which process comprises contacting osanetant free base with benzenesulfonic acid in a suitable solvent, wherein the solvent is not 4-methyl-2-pentanone. In certain embodiments, the osanetant free base is prepared by a process which does not comprise 4-methyl-2-pentanone. In certain embodiments, the osanetant benzenesulfonic acid Form 3 is prepared by a process which does not comprise 4-methyl-2-pentanone.

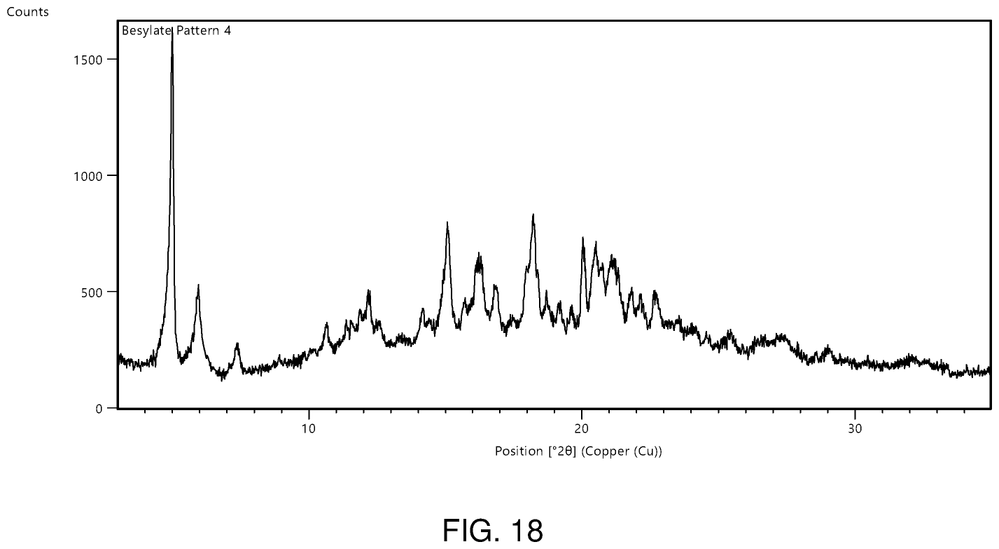

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide benzenesulfonic acid (osanetant benzenesulfonic acid Form 4) characterized by an X-ray powder diffractogram comprising the following peaks: 5.0°2θ±0.2°2θ, 15.1°2θ±0.2°2θ, and 18.2°2θ±0.2°2θ. In certain embodiments, the diffractogram further comprises one or more additional peaks selected from 16.2°2θ±0.2°2θ, 20.0°2θ±0.2°2θ, 20.5°2θ±0.2°2θ, and 21.1°2θ±0.2°2θ. In certain embodiments, the diffractogram is substantially as shown in FIG. 18.

In one embodiment, provided is a solid form of (R)-N-(1-(3-(1-benzoyl-3-(3,4-dichlorophenyl)piperidin-3-yl)propyl)-4-phenylpiperidin-4-yl)-N-methylacetamide phosphoric acid (osanetant phosphoric acid). In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide phosphoric acid (osanetant phosphoric acid).

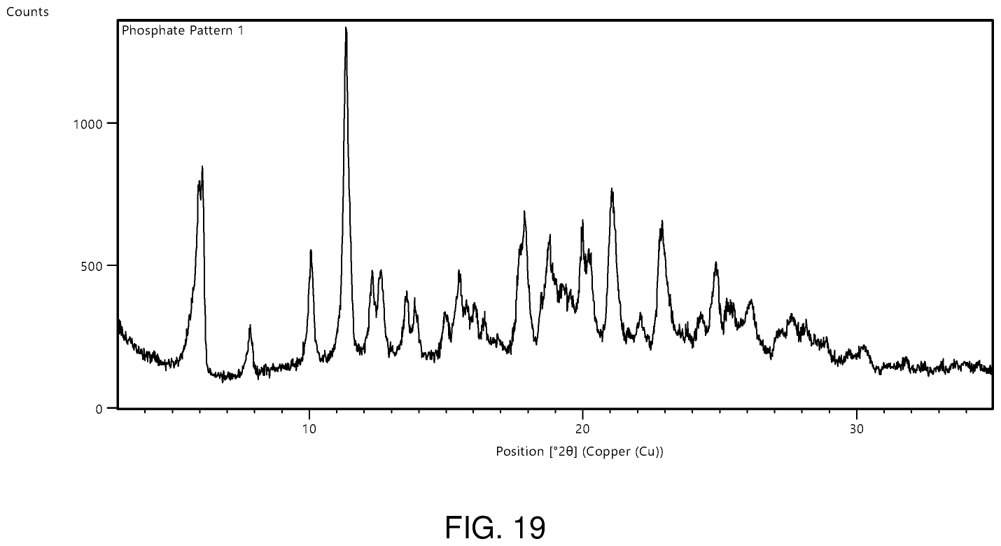

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide phosphoric acid (osanetant phosphoric acid Form 1) characterized by an X-ray powder diffractogram comprising the following peaks: 6.0°2θ±0.2°2θ, 6.1°2θ±0.2°2θ, and 11.3°2θ±0.2°2θ. In certain embodiments, the diffractogram further comprises one or more additional peaks selected from 17.9°2θ±0.2°2θ, 20.0°2θ±0.2°2θ, and 21.0°2θ±0.2°2θ. In certain embodiments, the diffractogram is substantially as shown in FIG. 19.

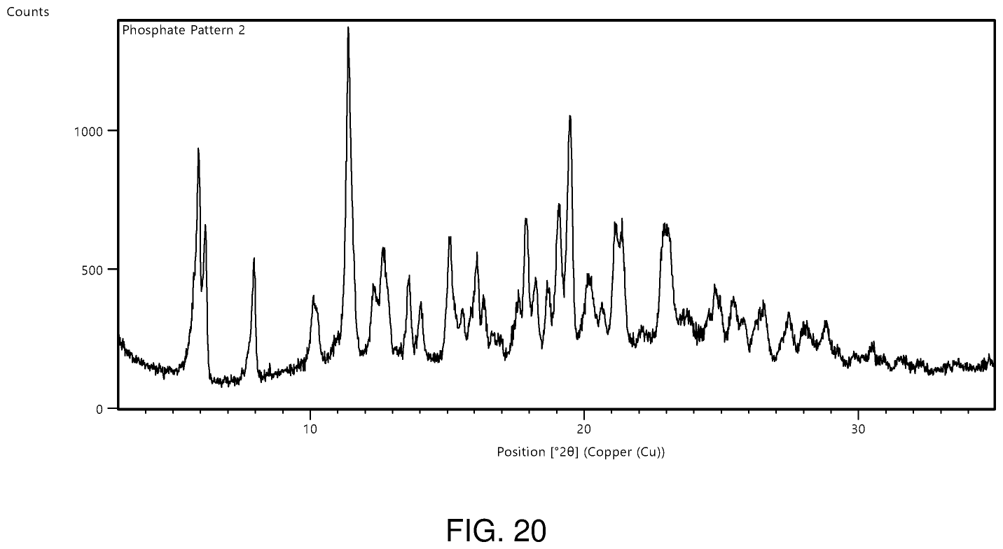

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide phosphoric acid (osanetant phosphoric acid Form 2) characterized by an X-ray powder diffractogram comprising the following peaks: 5.9°2θ±0.2°2θ, 11.4°2θ±0.2°2θ, and 19.5°2θ±0.2°2θ. In certain embodiments, the diffractogram further comprises one or more additional peaks selected from 6.2°2θ±0.2°2θ, 17.9°2θ±0.2°2θ, and 19.0°2θ±0.2°2θ. In certain embodiments, the diffractogram is substantially as shown in FIG. 20.

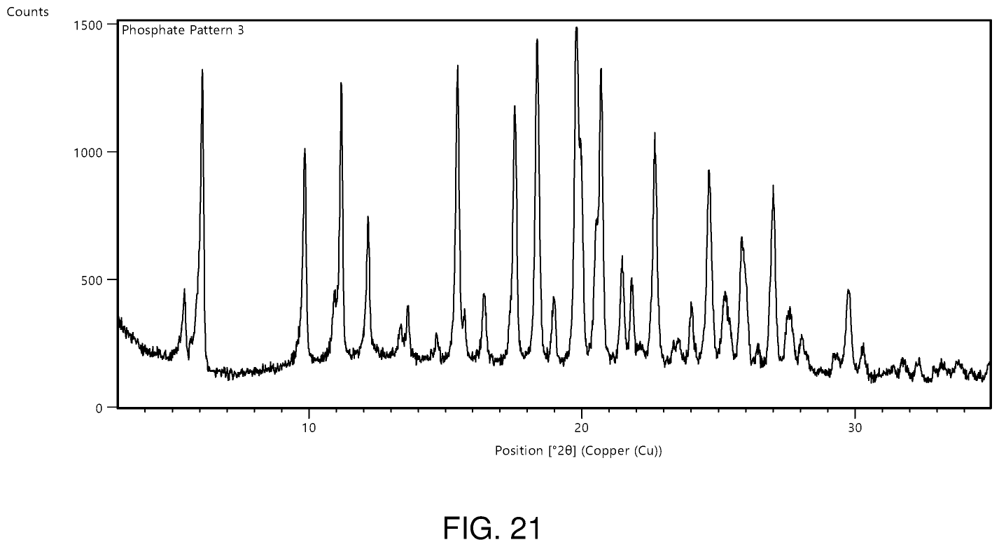

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide phosphoric acid (osanetant phosphoric acid Form 3) characterized by an X-ray powder diffractogram comprising the following peaks: 15.5°2θ±0.2°2θ, 18.4°2θ±0.2°2θ, and 19.8°2θ±0.2°2θ. In certain embodiments, the diffractogram further comprises one or more additional peaks selected from 6.1°2θ±0.2°2θ, 11.2°2θ±0.2°2θ, 17.5°2θ±0.2°2θ, and 20.7°2θ±0.2°2θ. In certain embodiments, the diffractogram is substantially as shown in FIG. 21.

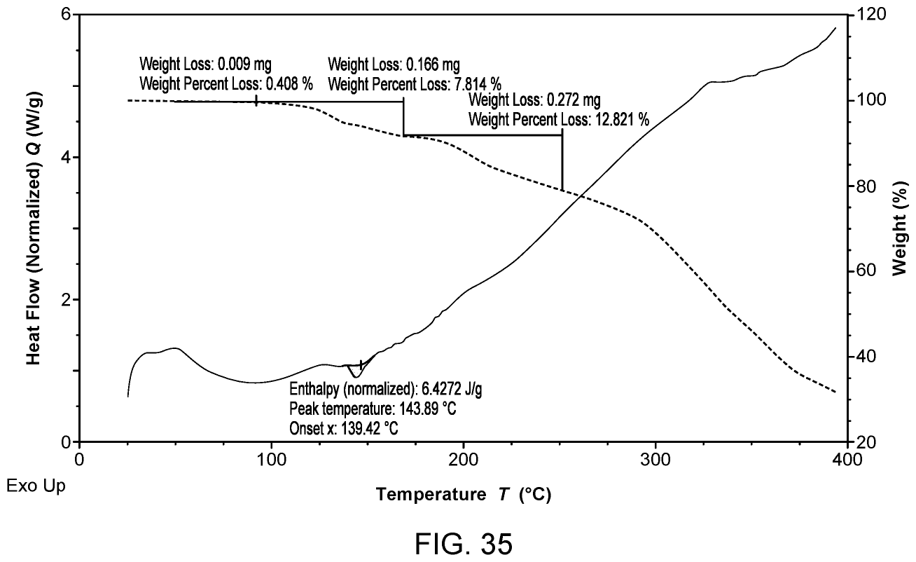

In certain embodiments, osanetant phosphoric acid Form 3 is characterized by a differential scanning calorimetry (DSC) curve that comprises a small endothermic event with an onset at about 139.4° C. (peak at about 143.9° C.). In certain embodiments, the DSC curve is substantially as shown in FIG. 35.

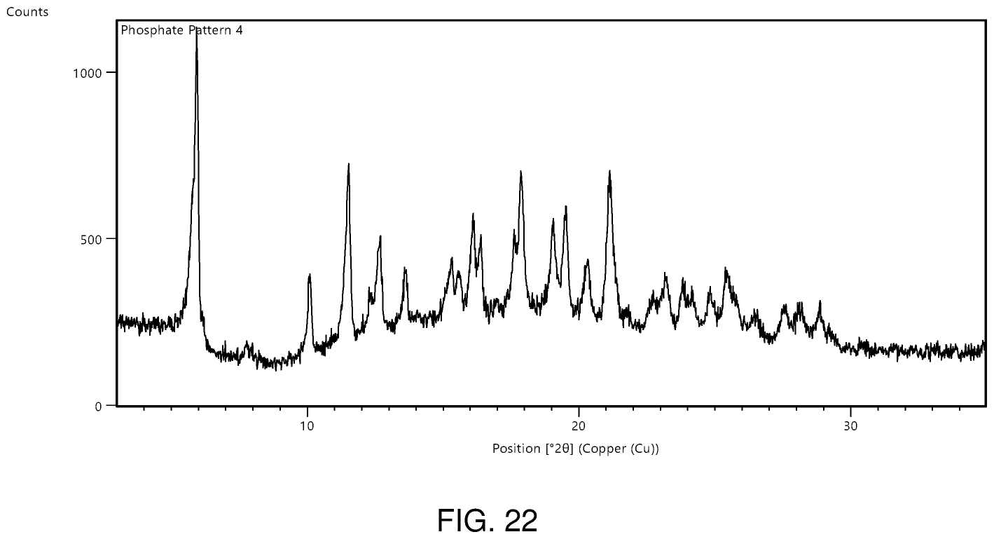

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide phosphoric acid (osanetant phosphoric acid Form 4) characterized by an X-ray powder diffractogram comprising the following peaks: 5.9°2θ±0.2°2θ, 11.5°2θ±0.2°2θ, and 17.9°2θ±0.2°2θ. In certain embodiments, the diffractogram further comprises one or more additional peaks selected from 16.1°2θ±0.2°2θ, 19.0°2θ±0.2°2θ, 19.5°2θ±0.2°2θ, and 21.1°2θ±0.2°2θ. In certain embodiments, the diffractogram is substantially as shown in FIG. 22.

In one embodiment, provided is a solid form of (R)-N-(1-(3-(1-benzoyl-3-(3,4-dichlorophenyl)piperidin-3-yl)propyl)-4-phenylpiperidin-4-yl)-N-methylacetamide L-malic acid (osanetant L-malic acid). In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide L-malic acid (osanetant L-malic acid).

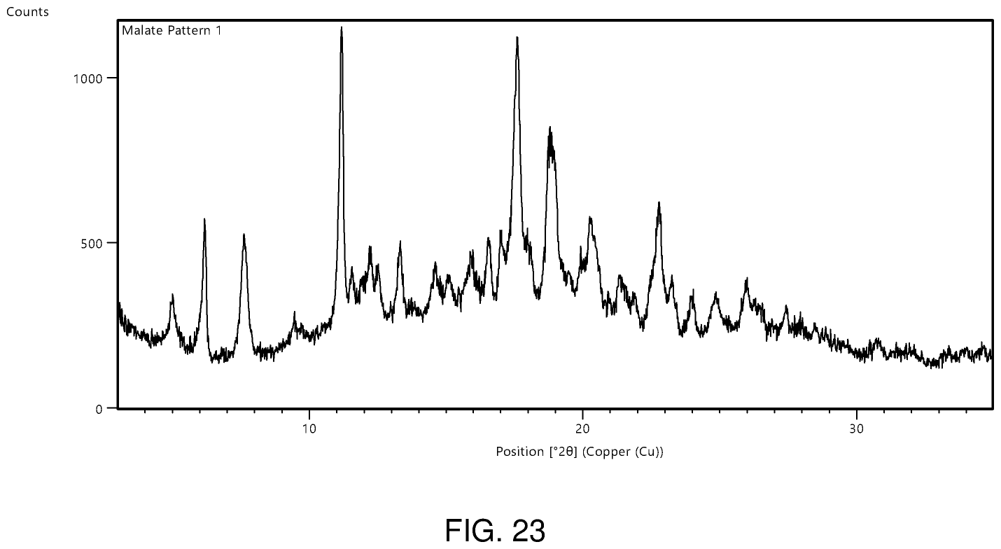

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide L-malic acid (osanetant L-malic acid Form 1) characterized by an X-ray powder diffractogram comprising the following peaks: 11.2°2θ±0.2°2θ, 17.6°2θ±0.2°2θ, and 18.8°2θ±0.2°2θ. In certain embodiments, the diffractogram further comprises one or more additional peaks selected from 6.2°2θ±0.2°2θ, 19.0°2θ±0.2°2θ, and 22.8°2θ±0.2°2θ. In certain embodiments, the diffractogram is substantially as shown in FIG. 23.

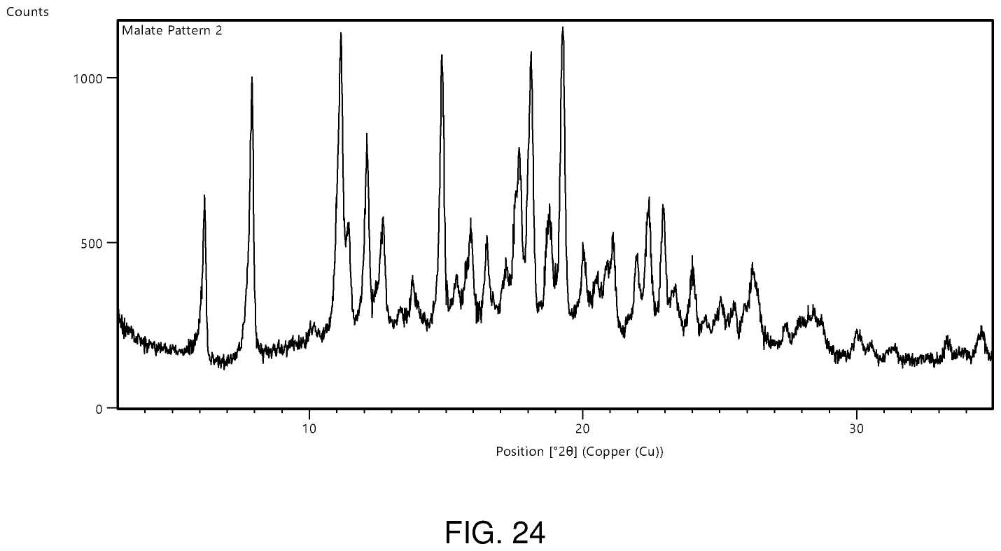

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide L-malic acid (osanetant L-malic acid Form 2) characterized by an X-ray powder diffractogram comprising the following peaks: 11.2°2θ±0.2°2θ, 14.8°2θ±0.2°2θ, and 19.3°2θ±0.2°2θ. In certain embodiments, the diffractogram further comprises one or more additional peaks selected from 8.0°2θ±0.2°2θ, 12.1°2θ±0.2°2θ, and 18.1°2θ±0.2°2θ. In certain embodiments, the diffractogram is substantially as shown in FIG. 24.

In one embodiment, provided is a solid form of (R)-N-(1-(3-(1-benzoyl-3-(3,4-dichlorophenyl)piperidin-3-yl)propyl)-4-phenylpiperidin-4-yl)-N-methylacetamide benzoic acid (osanetant benzoic acid). In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide benzoic acid (osanetant benzoic acid).

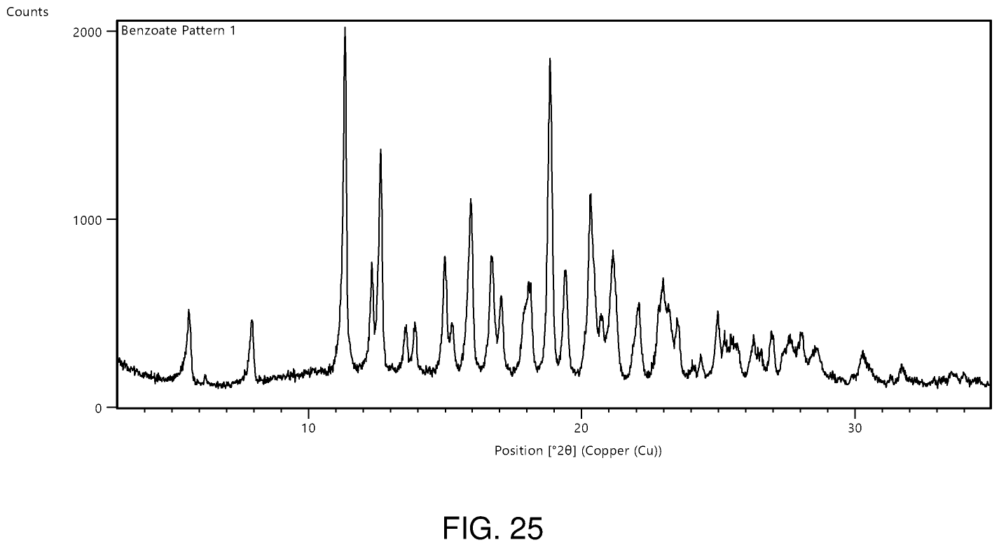

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide benzoic acid (osanetant benzoic acid Form 1) characterized by an X-ray powder diffractogram comprising the following peaks: 11.3°2θ±0.2°2θ, 12.7°2θ±0.2°2θ, and 18.8°2θ±0.2°2θ. In certain embodiments, the diffractogram further comprises one or more additional peaks selected from 15.9°2θ±0.2°2θ, 20.3°2θ±0.2°2θ, and 21.2°2θ±0.2°2θ. In certain embodiments, the diffractogram is substantially as shown in FIG. 25.

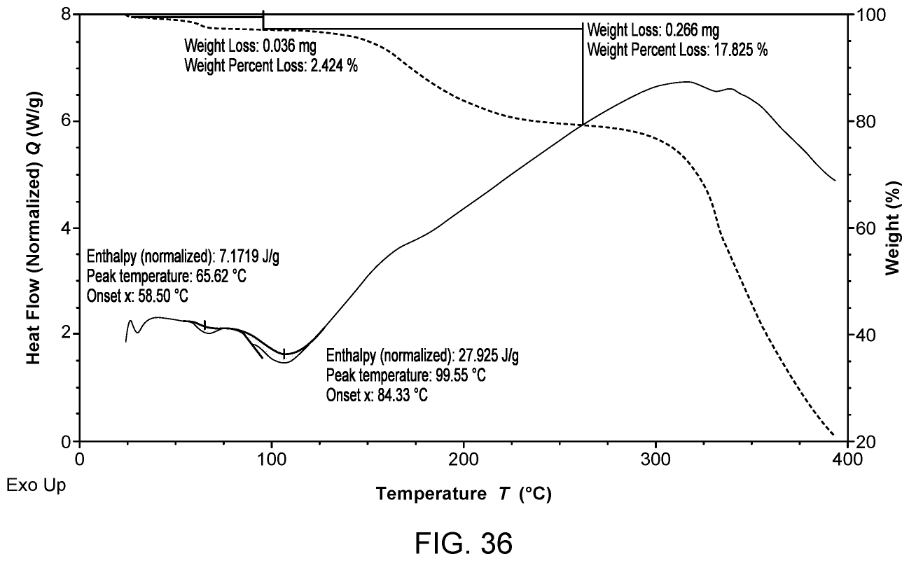

In certain embodiments, osanetant benzoic acid Form 1 is characterized by a differential scanning calorimetry (DSC) curve that comprises a small endothermic event with a peak at about 65.6° C. and a broad overlapping endothermic events with a peak at about 105° C. In certain embodiments, the DSC curve is substantially as shown in FIG. 36.

In one embodiment, provided is a solid form of (R)-N-(1-(3-(1-benzoyl-3-(3,4-dichlorophenyl)piperidin-3-yl)propyl)-4-phenylpiperidin-4-yl)-N-methylacetamide acetic acid (osanetant acetic acid). In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide acetic acid (osanetant acetic acid).

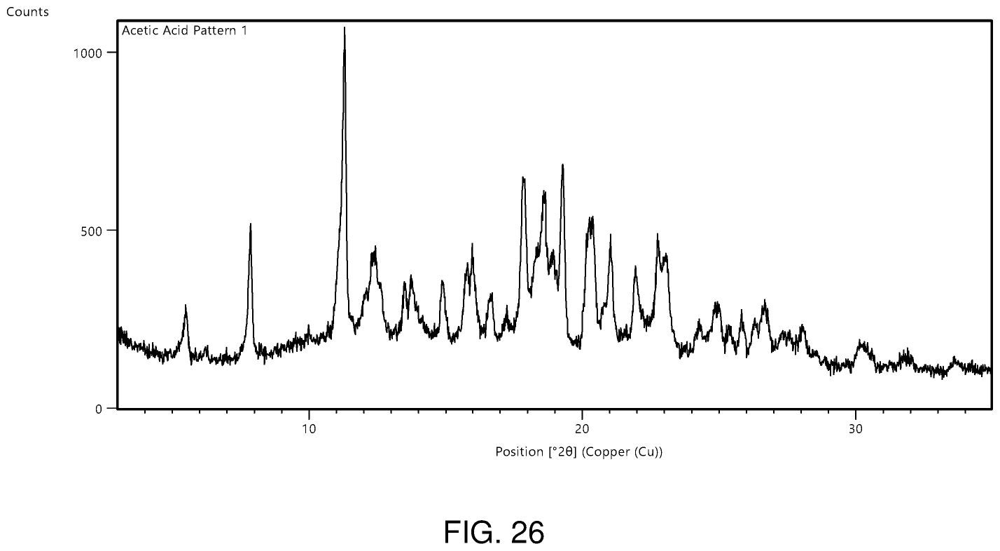

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide acetic acid (osanetant acetic acid Form 1) characterized by an X-ray powder diffractogram comprising the following peaks: 11.3°2θ±0.2°2θ, 17.8°2θ±0.2°2θ, and 19.3°2θ±0.2°2θ. In certain embodiments, the diffractogram further comprises one or more additional peaks selected from 7.9°2θ±0.2°2θ, 18.6°2θ±0.2°2θ, and 20.4°2θ±0.2°2θ. In certain embodiments, the diffractogram is substantially as shown in FIG. 26.

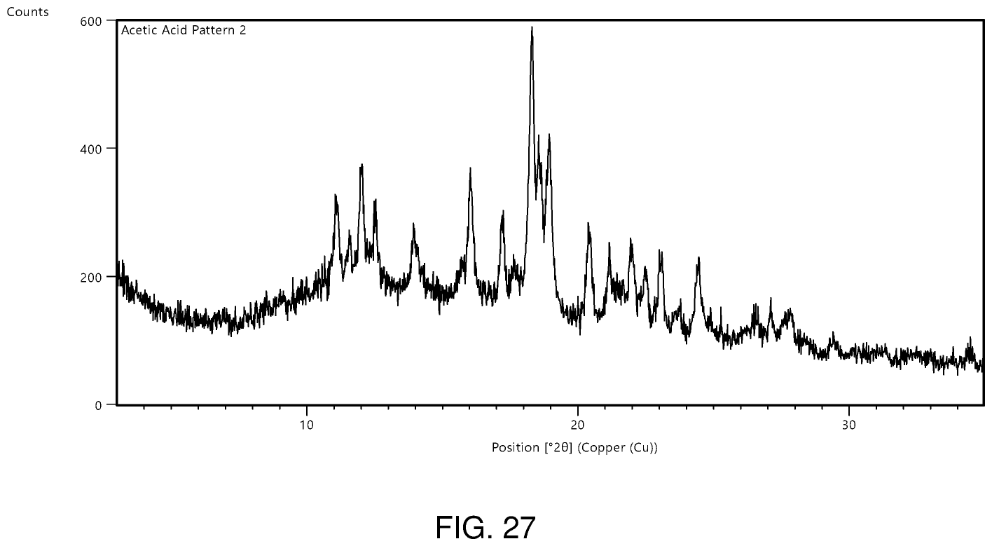

In one embodiment, provided is crystalline (R)-N-(1-(3-(1-benzoyl-3-(3,4-dichlorophenyl)piperidin-3-yl)propyl)-4-phenylpiperidin-4-yl)-N-methylacetamide acetic acid (osanetant acetic acid Form 2) characterized by an X-ray powder diffractogram comprising the following peaks: 12.0°2θ±0.2°2θ, 18.3°2θ±0.2°2θ, and 19.0°2θ±0.2°2θ. In certain embodiments, the diffractogram further comprises one or more additional peaks selected from 11.1°2θ±0.2°2θ, 16.0°2θ±0.2°2θ, and 17.2°2θ±0.2°2θ. In certain embodiments, the diffractogram is substantially as shown in FIG. 27.

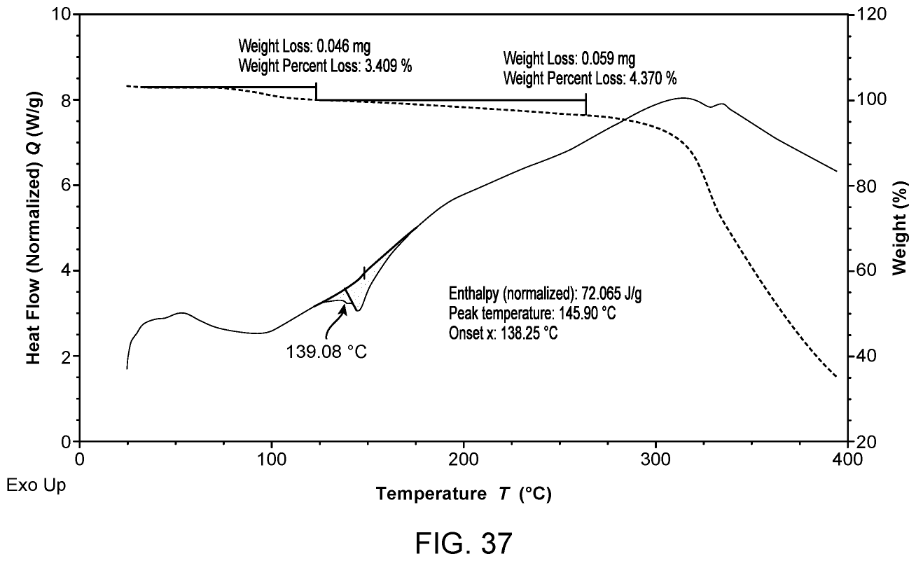

In certain embodiments, osanetant acetic acid Form 2 is characterized by a differential scanning calorimetry (DSC) curve that comprises an endothermic event with an onset at about 138.3° C. (peak at about 145.9° C.). In certain embodiments, the DSC curve is substantially as shown in FIG. 37.

### Methods and Uses

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes. including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

In one embodiment, provided is a method for treating, pretreating, or delaying onset or progression of a condition mediated at least in part by neurokinin 3 receptor antagonism, comprising administering to a patient in need thereof, a therapeutically effective amount of a crystalline salt form described herein, such as, but not limited to, osanetant methanesulfonic acid Form 1, osanetant methanesulfonic acid Form 2, osanetant sulfuric acid Form 1, osanetant sulfuric acid Form 2, osanetant ethanesulfonic acid Form 1, osanetant ethanesulfonic acid Form 2, osanetant ethane-1,2-disulfonic acid Form 1, osanetant p-toluenesulfonic acid Form 1, osanetant p-toluenesulfonic acid Form 2, osanetant p-toluenesulfonic acid Form 3, osanetant p-toluenesulfonic acid Form 4, osanetant p-toluenesulfonic acid Form 6, osanetant naphthalene-2-sulfonic acid Form 1, osanetant naphthalene-2-sulfonic acid Form 2, osanetant naphthalene-2-sulfonic acid Form 3, osanetant naphthalene-2-sulfonic acid Form 4, osanetant benzenesulfonic acid Form 1, osanetant benzenesulfonic acid Form 2, osanetant benzenesulfonic acid Form 3, osanetant benzenesulfonic acid Form 4, osanetant phosphoric acid Form 1, osanetant phosphoric acid Form 2, or osanetant phosphoric acid Form 3, or a composition comprising the same. In one embodiment, provided is a method for treating, pretreating, or delaying onset or progression of a condition mediated at least in part by neurokinin 3 receptor antagonism, comprising administering to a patient in need thereof, a therapeutically effective amount of a crystalline salt form described herein, such as, but not limited to, osanetant p-toluenesulfonic acid Form 1, osanetant p-toluenesulfonic acid Form 2, osanetant p-toluenesulfonic acid Form 3, osanetant p-toluenesulfonic acid Form 4, osanetant p-toluenesulfonic acid Form 6, osanetant benzenesulfonic acid Form 1, osanetant benzenesulfonic acid Form 2, osanetant benzenesulfonic acid Form 3, or osanetant benzenesulfonic acid Form 4, or a composition comprising the same.

In certain embodiments, the condition mediated at least in part by neurokinin 3 receptor antagonism is selected from the group consisting of a disease associated with a dysfunction of the dopaminergic systems, a disease associated with a dysfunction of the dopaminergic system, vigilance disorders, an epileptic disease, a neurodegenerative disease, a peripheral disease, cardiovascular disorders, rhythm disorders, respiratory disorders, disorders of the gastrointestinal system, stress-related disorders, disorders of the urinary system, acute or chronic inflammation, and diseases of the immune system.

In certain embodiments, the condition mediated at least in part by neurokinin 3 receptor antagonism is selected from the group consisting of schizophrenia, Parkinson's disease, anxiety, Grand Mal, dementia, pain, migraine, hypertension, cardiac insufficiency, asthma, rhinitis, cough, bronchitis, allergies, hypersensitivity, esophageal ulcer, colitis, irritable bowel syndrome (IBS), inflammatory bowel diseases (IBD), acidic secretion, incontinence, neurogenic bladder, and rheumatoid arthritis.

In certain embodiments, the condition mediated at least in part by neurokinin 3 receptor antagonism is selected from the group consisting of altered bowel function, androgen dependent acne, androgen-producing tumors, anxiety, atherosclerosis, atresia, anovulation, bladder dysfunction, benign prostatic hyperplasia (BPH), cancer, chronic traumatic encephalopathy, coronary artery disease, dysarthria, dysfunction of the dopaminergic systems, dysfunctional uterine bleeding, dysmenorrhea, dysphagia, eye movement difficulties, follicular maturation arrest, gastrointestinal dysfunction, HAIR-AN syndrome, hirsutism, hot flashes, hyperandrogenism, infertility, inflammation, inflammatory response, insomnia, loss of muscle coordination, loss of muscle function, macular degeneration, male pattern baldness, metastatic prostatic carcinoma, muscle wasting, nausea or vomiting, oculomotor gaze palsy, ovarian hyperthecosis, Parkinson's disease, peripheral vascular disease, polycystic ovary syndrome (PCOS), precocious puberty in boys, pre-eclampsia, PTSD, reduced dopaminergic neuron function, reducing intracranial pressure, reperfusion injury, respiratory depression, respiratory disorders, rheumatoid arthritis, supranuclear gaze palsy, testicular cancer, treating benign prostatic hyperplasia, treating mood disorders, treatment of excess body weight and/or excess body fat, varicose veins, vasomotor symptoms, virilization, and visceral pain. In certain embodiments, the osanetant solid forms described herein are used to treat any condition described herein by use of assays and/or methods described in, for example, WO2001095904, WO2002089802, WO2014089019, WO2015200594, WO2000043008. U.S. Pat. Nos. 20,060,281670, 7,947,458, 20,060,281670, which disclosure is incorporated herein by reference. In some embodiments, one or more of the salts described herein may have improved physical properties as compared to the earlier known salts, such as bioavailability, stability, purity, dissolution, low hygrscopicity and the like.

In some embodiments, the disclosure is related to treatment of conditions described herein provided that the condition is not anxiety and/or depression. Anxiety is a generalized response to an unknown threat or internal conflict. Anxiety may or may not be associated with specific cues. However, conditioned fear is focused on a known external danger and is cue-dependent. For example, the symptoms of conditioned fear include re-experiencing memories of traumatic events through intrusive thoughts, flashbacks, and nightmares. Persistent highly aversive memories related to the trauma, potentially over-consolidated memories, and the inability of these memories to be extinguished are all frequent characteristics of this disorder. Specifically relevant to conditioned fear is the memory consolidation phase following emotional learning (fear conditioning) since it is required to stabilize the initial fear memory trace.

In certain embodiments, the disclosure relates to a method of treating or preventing conditioned fear comprising administering an effective amount of a crystalline salt form described herein to a patient in need thereof. In certain embodiments, the disclosure relates to a method of treating or preventing acute stress disorder (ASD), comprising administering an effective amount of a crystalline salt form described herein to a patient in need thereof. Acute stress disorder refers to the initial traumatic symptoms that arise immediately after a traumatic event. Posttraumatic stress disorder (PTSD) generally refers to a time period in the aftermath of trauma. PTSD can follow after ASD, but it can also occur even when ASD does not develop. In certain embodiments, the patient is diagnosed with post-traumatic stress disorder. In some of such embodiments, the patient does not have a diagnosis of depression and/or anxiety.

In certain embodiments, the disclosure relates to a method for treating, preventing, or delaying onset or progression of post-traumatic stress disorder (PTSD), comprising administering to a patient in need thereof, a therapeutically effective amount of a crystalline salt form described herein or composition comprising the same. Post-traumatic stress disorder (PTSD) may develop following exposure to a traumatic event, and is characterized by (1) re-experiencing the traumatic event, such as recurrent nightmares, intrusive recollections of the event, flashbacks, physiological and psychological responses to internal or external cues relating to the event, etc.; (2) persistent avoidance of thoughts, people or places associated with the event; (3) numbing of general responsiveness such as emotional detachment, restricted affect or loss of interest in activities; and (4) persistence of increased arousal such as exaggerated startle response, hypervigilance, irritability, difficulty sleeping, etc. In certain embodiments, the disclosure contemplates a patient with conditioned fear as characterized by re-experiencing the traumatic event; e.g., recurrent nightmares, intrusive recollections of the event, flashbacks, recurring daily, bi-daily, or weekly; and one, two, or all of symptoms (2)-(4). In certain embodiments, the patient is at high-risk of PTSD such as a combat veteran, a victim of criminal mayhem, or a rape victim. In some of such embodiments, the patient does not have a diagnosis of depression and/or anxiety.

In certain embodiments, the disclosure relates to a method of treating conditioned fear induced by witnessing or experiencing a traumatic event, comprising administering to a patient in need thereof, a therapeutically effective amount of a crystalline salt form described herein or composition comprising the same. In certain embodiments, the patient exhibits one or more, or more than one, characteristic of the conditioned fear selected from re-experiencing the traumatic event; flashbacks; intrusive recollections; avoidance of thoughts, people or places associated with the event; numbing of general responsiveness; and increased arousal. In certain embodiments, the patient does not have a diagnosis of depression and/or anxiety.

In some embodiments, the therapeutically effective amount of a crystalline salt form described herein or composition comprising the same is provided prior to an event which may induce conditioned fear. In some embodiments, prior to comprises within 24 hours, 12 hours, three hours, two hours, or one hour prior to an event which may induce conditioned fear. In some embodiments, the therapeutically effective amount of a crystalline salt form described herein or a composition comprising the same is provided after an event which may or does induce a conditioned fear response. In some embodiments. after comprises within 24 hours, 12 hours, three hours, two hours, or one hour after an event which may or does induce a conditioned fear response.

In certain embodiments, the crystalline salt form described herein is administered within an hour of or within a day of experiencing a traumatic event.

In certain embodiments, the crystalline salt form described herein is administered within (before and/or after) an hour, two hours, three hours, 1 day, 2 days, or a week of experiencing a traumatic event. In certain embodiments, the traumatic event is that subject viewed the death of a human. In certain embodiments, the traumatic event is that subject viewed the body of a human in which at least one limb of the patient was separated from the body of a human.

In certain embodiments, the crystalline salt form described herein is administered at the time of or within (before and/or after) an hour, two hours, three hours, 1 day, 2 days, or a week of psychotherapy, cognitive behavioral therapy, exposure therapy, cognitive restructuring, or stress inoculation training.

In certain embodiments, the crystalline salt forms described herein are used in methods for treating a disorder selected from bone loss, post-traumatic stress disorder (PTSD), acute stress disorder, schizophrenia, bipolar disorder, schizoaffective disorder, premenstrual dysphoric disorder (PMDD), anxiety, depression, prostate cancer, polycystic ovarian syndrome (PCOS), and vasomotor symptoms. In some embodiments, the disorder is selected from post-traumatic stress disorder (PTSD), acute stress disorder, schizophrenia, anxiety, and depression. In some embodiments, the disorder is categorized as a DSM-5 disorder.

“DSM5 disorder” refers to any disorder described in the Diagnostic and Statistical Manual of Mental Disorders Fifth Edition, including and not limited to neurodevelopmental disorders, schizophrenia spectrum and other psychotic disorders, bipolar and related disorders, depressive disorders, anxiety disorders, obsessive-compulsive and related disorders, trauma-and stressor-related disorders, dissociative disorders, somatic symptom and related disorders, feeding and eating disorders, elimination disorders, sleep-wake disorders, sexual dysfunctions, gender dysphoria, disruptive, impulse-control, and conduct disorders, substance-related and addictive disorders, neurocognitive disorders, personality disorders, personality disorders, paraphilic disorders, other mental disorders, medication-induced movement disorders and/or other adverse effects of medication.

In some embodiments, a DSM5 disorder is schizoaffective disorder, bipolar type or depressive type with or without catatonia. In some embodiments, a DSM5 disorder is major depressive or bipolar disorder with psychotic or catatonic features. In some embodiments, a DSM5 disorder is schizophreniform disorder and brief psychotic disorder. In some embodiments, a DSM5 disorder is delusional disorder, schizotypal personality disorder, obsessive-compulsive disorder and body dysmorphic disorder, posttraumatic stress disorder, autism spectrum disorder or communication disorders, or other mental disorders associated with a psychotic episode. In some embodiments, a DSM5 disorder is substance/medication-induced psychotic disorder with delusions and/or hallucinations. In some embodiments, a DSM5 disorder is a manic episode, a hypomanic episode, or a major depressive episode. In some embodiments, a DSM5 disorder is generalized anxiety disorder, panic disorder, posttraumatic stress disorder, or other anxiety disorder. In some embodiments, a DSM5 disorder is substance/medication-induced bipolar disorder, or Attention-deficit/hyperactivity disorder. In some embodiments, a DSM5 disorder is acute anxiety, or medication induced manic or depressive or manic disorder.

In some embodiments, a DSM5 disorder is trauma-and stressor-related disorders including and not limited to disorders in which exposure to a traumatic or stressful event is implicated, reactive attachment disorder, disinhibited social engagement disorder, posttraumatic stress disorder (PTSD), PTSD with dissociative symptoms, acute stress disorder, and adjustment disorders.

In some embodiments a DSM5 disorder is a mood disorder. In some embodiments a DSM5 disorder is a mood disorder with peripartum onset. In some embodiments, a DSM5 disorder is a depressive disorder including and not limited to disruptive mood dysregulation disorder, major depressive disorder (including major depressive episode), persistent depressive disorder (dysthymia). premenstrual dysphoric disorder (PMDD), substance/medication-induced depressive disorder, depressive disorder due to another medical condition, other specified depressive disorder, and unspecified depressive disorder. In some embodiments, a DSM5 disorder is recurrent brief depression, short-duration depressive episode, or peripartum/postpartum depression. In some embodiments a DSM5 disorder is seasonal affective disorder (SAD), atypical depression, psychotic depression, situational depression or treatment resistant depression. In some embodiments, a DSM5 disorder is generalized anxiety disorder, social anxiety disorder, or panic disorder. In some embodiments, a DSM5 disorder is anxiety disorder due to another medical condition, substance/medication-induced anxiety disorder, anxiety or depression due to other medical condition (e.g., eating disorders). In some embodiments, a DSM5 disorder is obsessive compulsive disorder, or body dysmorphic disorder.

The methods of the disclosure encompass the use of any type of psychotherapy that is suitable and may be conducted in one or more sessions. Suitable methods of psychotherapy include behavior psychotherapy such as exposure-based psychotherapy, cognitive psychotherapy including cognitive training and psychodynamically oriented psychotherapy (see, for example, Foa (2000) J. Clin. Psych. 61 (suppl. 5): 43-38).

“Psychotherapy” refers broadly to forms of psychiatric treatment which employ specialized communication techniques practiced by a properly trained physician, counselor, or clinician for the purpose of curing or reducing or alleviating a behavioral disorder of a patient and improving the patient's emotional, social, and/or mental health.

Provided herein is a method of preventing, including blocking, attenuating, or limiting, the development of one or more vasomotor symptoms (VMS) in a patient. In some embodiments, blocking, attenuating, or limiting the development of one or more vasomotor symptoms comprises treating one or more of flushing of the skin, sweating, palpitations, racing heart rate, shivering, hot flashes, chills, irritability, anxiety, mood disorders, or depression. In some embodiments, the one or more vasomotor symptoms is hot flashes.

One of the most broadly experienced symptoms in women and men who have undergone hormone deprivation therapy, and/or medical or surgical interventions relating to cancer or prevention of cancer is vasomotor symptoms (VMS). In some instances, this is referred to as induced VMS or iVMS. VMS, such as hot flashes and night sweats, is characterized by a sudden feeling of warmth, usually localized in the face, neck, and chest, and is accompanied by sweating and flushing of the skin. The discomfort associated with these symptoms can negatively impact sufferers' ability to achieve restful sleep, and generally diminish quality of life. In certain embodiments, the patient will be undergoing hormone deprivation therapy, a medical and/or surgical procedure that may cause VMS, comprising administering an effective amount of a crystalline salt form described herein, for a time period prior to, or concurrently with, the hormone deprivation therapy, a medical and/or surgical procedure. It is contemplated that, in certain embodiments, treating with a crystalline salt form of osanetant as described herein prior to the surgery or hormone deprivation therapy will prevent the hypertrophy of the KNDy neurons, thereby leading to a lower effective dose of a crystalline salt form of osanetant as described herein compared to post-menopausal VMS.

In certain embodiments, the condition to be treated is induced Vasomotor Symptoms (iVMS). In certain embodiments, the induced Vasomotor Symptoms (iVMS) is a result of one or more of hormone deprivation, surgery, or from treatment with tamoxifen or leuprolide.

In certain embodiments, the patient has cancer, has had cancer, or has an increased risk for cancer. In some embodiments, the cancer is breast cancer, ovarian cancer, uterine cancer, testicular, or prostate cancer. In some embodiments, the cancer is breast cancer. In some embodiments, the cancer is metastatic breast cancer. In some embodiments, the cancer is ovarian cancer. In some embodiments, the cancer is uterine cancer. In some embodiments, the cancer is prostate cancer. In some embodiments, the cancer is hormone receptor-positive cancer, e.g., breast cancer or prostate cancer.

In some embodiments, the patient has suffered from cancer but is in remission. In some embodiments, the patient has an increased risk for cancer. In some embodiments, such a risk may be from a genetic mutation implicated in cancer. In some embodiments, the patient has tested positive for a BRCA1, BRCA2, or PALB2 mutation. In some embodiments, the patient has tested positive for a BRCA1 mutation. In some embodiments, the patient has tested positive for a BRCA2 mutation. In some embodiments, the patient has tested positive for a PALB2 mutation. In some embodiments, the patient is menopausal. In some embodiments, the patient is perimenopausal.

Inherited mutations to certain genes can lead to the development of specific types of cancers. BRCA1 and BRCA2 are genes that code for tumor suppressor proteins, which assist in repairing damage to DNA. When an individual has a mutated version of either gene, the individual will not produce a functional form of the related tumor suppressor protein, so DNA damage will accumulate, which can lead to the development of cancer. Mutations in BRCA1 and BRCA2 have been shown to increase the risk for the development of ovarian and fallopian tube cancer in women, prostate cancer in men, and, most notably, breast cancer in both women and men. In addition to BRCA1 and 2 mutations, patients having a mutation in PALB2 are also at risk for increase of cancer. In some embodiments provided herein, the genetic predisposition to developing certain mutation-linked or hormone-dependent cancers is having tested positive for a BRCA1 mutation. In some embodiments provided herein, the genetic predisposition to developing certain mutation-linked or hormone-dependent cancers is having tested positive for a BRCA2 mutation. In some embodiments provided herein, the genetic predisposition to developing certain mutation-linked or hormone-dependent cancers is having tested positive for a PALB2 mutation.

Given the significant increase in risk in developing these specific cancers associated with possessing a mutant BRCA1, BRCA2 or PALB2 gene, many women who are carriers opt for the prophylactic removal of breast tissue (mastectomy), fallopian tubes (salpingectomy), uterus (hysterectomy) and/or ovaries (oophorectomy), to stem the possibility of developing an associated cancer in these organs, as well as to limit the presence of the endogenous sex hormones that cause the proliferation of hormone-dependent cancers. In certain embodiments, the patient to be treated possess the BRCA1, BRCA2 or PALB2 gene and are having their breast tissue (mastectomy), fallopian tubes (salpingectomy), uterus (hysterectomy) and/or ovaries (oophorectomy) removed.

For men suffering from prostate cancer, surgical resection of the prostate (prostatectomy), seminal vesicles, and neighboring lymph nodes, as well as androgen suppression or deprivation therapy are common treatments for the disease. In situations of advanced prostate cancer, removal of the testes (orchiectomy), the primary source of endogenous testosterone in the male body, can be undertaken to further limit the growth and spread of the hormone-dependent prostate cancer.

For hormone-dependent cancers, such as breast, ovarian, uterine, prostate, and testicular cancers, the proliferation of cells is driven by hormone-receptor interactions on cell surfaces. In the presence of these sex hormones, namely estrogen, progesterone, and testosterone, the hormone-dependent cells replicate more frequently, increasing the opportunity for genetic errors to occur and accumulate, potentially leading to cancer. Pharmaceutical interventions, e.g. hormone deprivation therapy, for the treatment or prevention of hormone-dependent cancers include compounds that inhibit the synthesis of these sex hormones, such as gonadotropin-releasing hormone (GnRH) agonists and antagonists, compounds which block receptor sites on hormone-dependent cancer cell surfaces, such as selective estrogen-receptor modulators (SERMs) or nonsteroidal antiandrogens (NSAAs), and selective estrogen receptor degraders (SERDS). In certain embodiments, the patient will be undergoing hormone deprivation therapy. For instance, in the case of prostate cancer, the patient may be undergoing androgen deprivation therapy.

Many patients opt for medical or surgical interventions to remove the cancer and surrounding tissues. Many patients also elect for the removal of sex hormone producing organs, namely the ovaries or testes. In certain embodiments, the patient to be treated will be undergoing removal of ovaries or testes. In certain embodiments, the patient will already be receiving a GnRH agonist or antagonist, e.g. leuprolide. In certain embodiments, the patient will already be receiving a SERM, e.g. tamoxifen.

In some embodiments, the hormone deprivation therapy, and/or medical or surgical procedure that may cause VMS is the prophylactic removal of breast tissue (mastectomy). In some embodiments, the hormone deprivation therapy, and/or medical or surgical procedure that may cause VMS is the prophylactic removal of the fallopian tubes (salpingectomy). In some embodiments, the hormone deprivation therapy, and/or medical or surgical procedure that may cause VMS is the prophylactic removal of the ovaries (oophorectomy). In some embodiments, the hormone deprivation therapy, and/or medical or surgical procedure that may cause VMS is the prophylactic removal of one or more of breast tissue, fallopian tubes, and/or ovaries.

In some embodiments, the hormone deprivation therapy, and/or medical or surgical procedure that may cause VMS is the removal of the prostate (prostatectomy). In some embodiments, the hormone deprivation therapy, and/or medical or surgical procedure that may cause VMS is the removal of the seminal vesicles. In some embodiments, the hormone deprivation therapy, and/or medical or surgical procedure that may cause VMS is the removal of the testes (orchiectomy). In some embodiments, the hormone deprivation therapy, and/or medical or surgical procedure that may cause VMS is the administration of antiandrogen drugs. In some embodiments, the hormone deprivation therapy, and/or medical or surgical procedure that may cause VMS is the removal of the prostate, seminal vesicles, one or more testes, and/or the administration of antiandrogen drugs.

For decades, hormone replacement therapy (HRT), in which patients are given estrogen or an estrogen-progestin combination, has been prescribed to women to help ease their menopausal symptoms, including VMS. However, studies showing women who were treated with HRT for their menopausal symptoms, including VMS, had a higher incidence of certain hormone-dependent cancers, which has led medical professionals to reevaluate the risks associated with the practice. In patients who are already at an increased genetic risk for developing certain cancers, such as those with BRCA1 or BRCA2 mutations who may have undergone prophylactic surgeries to avoid the development of cancer in the previous mentioned organs, the risks associated with HRT treatment for VMS heavily outweigh the potential benefits. As such, in certain embodiments, HRT is contra-indicated.

In some embodiments provided herein, the hormone therapy is estrogen therapy. In some embodiments provided herein, the hormone therapy is an estrogen and progestin combination therapy. In some embodiments provided herein, the hormone therapy is tibolone therapy.

In some embodiments, for any method described herein, hormone therapy for the patient is contraindicated.

In some embodiments, the hormone therapy is estrogen therapy. In some embodiments, the hormone deprivation therapy is treatment with a selective estrogen receptor modulator (SERM). In some embodiments, the SERM is tamoxifen.

In some embodiments, the patient is a female patient. In some embodiments, the patient is a post-menopausal female patient.

In some embodiments, the hormone deprivation therapy is treatment with a gonadotropin-releasing hormone (GnRH) agonist or antagonist. In some embodiments, the patient is a male patient. In some embodiments, the GnRH agonist is leuprolide.

In some embodiments, the hormone deprivation therapy is treatment with a selective estrogen receptor degrader (SERD).

In some embodiments, the cancer is breast cancer, ovarian cancer, uterine cancer, or prostate cancer. In some embodiments, the cancer is hormone receptor-positive cancer. In some embodiments, the cancer is breast cancer. In some embodiments, the cancer is prostate cancer. In some embodiments, the patient has tested positive for a BRCA1, BRCA2 or PALB2 mutation.

In some embodiments, provided herein is a method for reducing the frequency and severity of hormone deprivation therapy-induced vasomotor symptoms or surgery-induced vasomotor symptoms in a cancer patient, the method comprising administering a combination of a hormone antagonist and a crystalline salt form of osanetant as described herein to the cancer patient in need thereof.

In some embodiments, provided herein is a method for reducing the frequency and severity of tamoxifen-induced vasomotor symptoms or surgery-induced vasomotor symptoms in a cancer patient, the method comprising administering a combination of tamoxifen and a crystalline salt form of osanetant as described herein to the cancer patient in need thereof.

Provided herein is a method for reducing leuprolide-induced vasomotor symptoms or surgery-induced vasomotor symptoms in a cancer patient, the method comprising administering a combination of leuprolide and a crystalline salt form of osanetant as described herein to the cancer patient in need thereof.

In some embodiments, the crystalline salt form of osanetant as described herein is administered to a patient for a time period prior to the hormone deprivation therapy, and/or medical or surgical procedure as described herein. In some embodiments, the crystalline salt form of osanetant as described herein is administered concurrently with hormone deprivation therapy, a medical and/or surgical procedure. In some embodiments, the patient continues to receive a crystalline salt form of osanetant as described herein after the hormone deprivation therapy, a medical and/or surgical procedure. In some embodiments, the patient receives a crystalline salt form of osanetant as described herein after short-term (e.g., 1 to 6 months, 1 to 3 months) hormone deprivation therapy. Any combination of these therapeutic regimens is contemplated within the scope of embodiments presented herein.

In some embodiments, the administration of a neurokinin receptor antagonist confers an additional benefit and reduces or eliminates social isolation stress (SIS) in a cancer patient, thereby improving prognosis (e.g., lifespan, regression of the cancer, and/or quality of life) for cancer patients. Accordingly in any embodiment of any method described above and herein, the method further provides for alleviation of social isolation stress (SIS) in the cancer patient.

### Pharmaceutical Compositions

In some embodiments, provided is a composition comprising osanetant, wherein at least about 85% of the osanetant present in the composition is a single crystalline salt form as disclosed herein. The osanetant present in the composition can be any one or more solid forms of osanetant, such as, but not limited to, another crystalline salt form of osanetant, a crystalline form of osanetant free base, an amorphous form of osanetant free base or salt thereof, etc. In some embodiments, provided is a composition comprising osanetant, wherein at least about 85% of the osanetant present in the composition is a crystalline salt form selected from the group consisting of osanetant methanesulfonic acid Form 1, osanetant methanesulfonic acid Form 2, osanetant sulfuric acid Form 1, osanetant sulfuric acid Form 2, osanetant ethanesulfonic acid Form 1, osanetant ethanesulfonic acid Form 2, osanetant p-toluenesulfonic acid Form 1, osanetant p-toluenesulfonic acid Form 2, osanetant p-toluenesulfonic acid Form 3, osanetant p-toluenesulfonic acid Form 4, osanetant p-toluenesulfonic acid Form 6, osanetant naphthalene-2-sulfonic acid Form 1, osanetant naphthalene-2-sulfonic acid Form 2, osanetant naphthalene-2-sulfonic acid Form 3, osanetant naphthalene-2-sulfonic acid Form 4, osanetant benzenesulfonic acid Form 1, osanetant benzenesulfonic acid Form 2, osanetant benzenesulfonic acid Form 3, osanetant benzenesulfonic acid Form 4, osanetant phosphoric acid Form 1, osanetant phosphoric acid Form 2, osanetant phosphoric acid Form 3, osanetant phosphoric acid Form 4, osanetant L-malic acid Form 1, osanetant L-malic acid Form 2, osanetant benzoic acid Form 1, osanetant acetic acid Form 1, and osanetant free base Form 2.

In some embodiments, provided is a composition comprising osanetant, wherein at least about 85% of the osanetant present in the composition is a single crystalline salt form as disclosed herein. In some embodiments, provided is a composition comprising osanetant, wherein at least about 90% of the osanetant present in the composition is a single crystalline salt form as disclosed herein. In some embodiments, provided is a composition comprising osanetant, wherein at least about 95% of the osanetant present in the composition is a single crystalline salt form as disclosed herein. In some embodiments, provided is a composition comprising osanetant, wherein at least about 97% of the osanetant present in the composition is a single crystalline salt form as disclosed herein. In some embodiments, provided is a composition comprising osanetant, wherein at least about 98% of the osanetant present in the composition is a single crystalline salt form as disclosed herein. In some embodiments, provided is a composition comprising osanetant, wherein at least about 99% of the osanetant present in the composition is a single crystalline salt form as disclosed herein. In some embodiments, provided is a composition comprising osanetant, wherein at least about 99.5% of the osanetant present in the composition is a single crystalline salt form as disclosed herein. In some embodiments, provided is a composition comprising osanetant, wherein at least about 99.9% of the osanetant present in the composition is a single crystalline salt form as disclosed herein. In some embodiments, provided is a composition comprising osanetant, wherein at least about 99.9% of the osanetant present in the composition is a single crystalline salt form as disclosed herein.

In certain embodiments, provided is a composition comprising osanetant, wherein at least about 85% of the osanetant present in the composition is osanetant naphthalene-2-sulfonic acid Form 3. In certain embodiments, provided is a composition comprising osanetant, wherein at least about 90% of the osanetant present in the composition is osanetant naphthalene-2-sulfonic acid Form 3. In certain embodiments, provided is a composition comprising osanetant, wherein at least about 95% of the osanetant present in the composition is osanetant naphthalene-2-sulfonic acid Form 3. In certain embodiments, provided is a composition comprising osanetant, wherein at least about 97% of the osanetant present in the composition is osanetant naphthalene-2-sulfonic acid Form 3. In certain embodiments, provided is a composition comprising osanetant, wherein at least about 98% of the osanetant present in the composition is osanetant naphthalene-2-sulfonic acid Form 3. In certain embodiments, provided is a composition comprising osanetant, wherein at least about 99% of the osanetant present in the composition is osanetant naphthalene-2-sulfonic acid Form 3. In certain embodiments, provided is a composition comprising osanetant, wherein at least about 99.5% of the osanetant present in the composition is osanetant naphthalene-2-sulfonic acid Form 3. In certain embodiments, provided is a composition comprising osanetant, wherein at least about 99.9% of the osanetant present in the composition is osanetant naphthalene-2-sulfonic acid Form 3. In certain embodiments, provided is a composition comprising osanetant, wherein at least about 99.99% of the osanetant present in the composition is osanetant naphthalene-2-sulfonic acid Form 3.

In certain embodiments, provided is a composition comprising osanetant, wherein at least about 85% of the osanetant present in the composition is osanetant benzenesulfonic acid Form 1. In certain embodiments, provided is a composition comprising osanetant, wherein at least about 90% of the osanetant present in the composition is osanetant benzenesulfonic acid Form 1. In certain embodiments, provided is a composition comprising osanetant, wherein at least about 95% of the osanetant present in the composition is osanetant benzenesulfonic acid Form 1. In certain embodiments, provided is a composition comprising osanetant, wherein at least about 97% of the osanetant present in the composition is osanetant benzenesulfonic acid Form 1. In certain embodiments, provided is a composition comprising osanetant, wherein at least about 98% of the osanetant present in the composition is osanetant benzenesulfonic acid Form 1. In certain embodiments, provided is a composition comprising osanetant, wherein at least about 99% of the osanetant present in the composition is osanetant benzenesulfonic acid Form 1. In certain embodiments, provided is a composition comprising osanetant, wherein at least about 99.5% of the osanetant present in the composition is osanetant benzenesulfonic acid Form 1. In certain embodiments, provided is a composition comprising osanetant, wherein at least about 99.9% of the osanetant present in the composition is osanetant benzenesulfonic acid Form 1. In certain embodiments, provided is a composition comprising osanetant, wherein at least about 99.99% of the osanetant present in the composition is osanetant benzenesulfonic acid Form 1. In certain embodiments, the composition comprising osanetant benzenesulfonic acid Form 1 does not comprise 4-methyl-2-pentanone or a osanetant benzenesulfonic acid 4-methyl-2-pentanone solvate.

In certain embodiments, provided is a composition comprising osanetant, wherein at least about 85% of the osanetant present in the composition is osanetant benzenesulfonic acid Form 3. In certain embodiments, provided is a composition comprising osanetant, wherein at least about 90% of the osanetant present in the composition is osanetant benzenesulfonic acid Form 3. In certain embodiments, provided is a composition comprising osanetant, wherein at least about 95% of the osanetant present in the composition is osanetant benzenesulfonic acid Form 3. In certain embodiments, provided is a composition comprising osanetant, wherein at least about 97% of the osanetant present in the composition is osanetant benzenesulfonic acid Form 3. In certain embodiments, provided is a composition comprising osanetant, wherein at least about 98% of the osanetant present in the composition is osanetant benzenesulfonic acid Form 3. In certain embodiments, provided is a composition comprising osanetant, wherein at least about 99% of the osanetant present in the composition is osanetant benzenesulfonic acid Form 3. In certain embodiments, provided is a composition comprising osanetant, wherein at least about 99.5% of the osanetant present in the composition is osanetant benzenesulfonic acid Form 3. In certain embodiments, provided is a composition comprising osanetant, wherein at least about 99.9% of the osanetant present in the composition is osanetant benzenesulfonic acid Form 3. In certain embodiments, provided is a composition comprising osanetant, wherein at least about 99.99% of the osanetant present in the composition is osanetant benzenesulfonic acid Form 3. In certain embodiments, the composition comprising osanetant benzenesulfonic acid Form 3 does not comprise 4-methyl-2-pentanone or a osanetant benzenesulfonic acid 4-methyl-2-pentanone solvate.

In certain embodiments, provided is a composition comprising osanetant benzenesulfonic acid Form 1 and osanetant benzenesulfonic acid Form 3. In certain embodiments, at least about 85% of the osanetant present in the composition is osanetant benzenesulfonic acid Form 3. In certain embodiments, at least about 90% of the osanetant present in the composition is osanetant benzenesulfonic acid Form 3. In certain embodiments, at least about 95% of the osanetant present in the composition is osanetant benzenesulfonic acid Form 3. In certain embodiments, at least about 97% of the osanetant present in the composition is osanetant benzenesulfonic acid Form 3. In certain embodiments, at least about 98% of the osanetant present in the composition is osanetant benzenesulfonic acid Form 3. In certain embodiments, at least about 99% of the osanetant present in the composition is osanetant benzenesulfonic acid Form 3. In certain embodiments, at least about 99.5% of the osanetant present in the composition is osanetant benzenesulfonic acid Form 3. In certain embodiments, at least about 99.9% of the osanetant present in the composition is osanetant benzenesulfonic acid Form 3. In certain embodiments, at least about 99.99% of the osanetant present in the composition is osanetant benzenesulfonic acid Form 3. In certain embodiments, the composition does not comprise 4-methyl-2-pentanone or an osanetant benzenesulfonic acid 4-methyl-2-pentanone solvate.

In certain embodiments, provided is a composition comprising osanetant, wherein at least about 85% of the osanetant present in the composition is osanetant p-toluenesulfonic acid Form 1. In certain embodiments, provided is a composition comprising osanetant, wherein at least about 90% of the osanetant present in the composition is osanetant p-toluenesulfonic acid Form 1. In certain embodiments, provided is a composition comprising osanetant, wherein at least about 95% of the osanetant present in the composition is osanetant p-toluenesulfonic acid Form 1. In certain embodiments, provided is a composition comprising osanetant, wherein at least about 97% of the osanetant present in the composition is osanetant p-toluenesulfonic acid Form 1. In certain embodiments, provided is a composition comprising osanetant, wherein at least about 98% of the osanetant present in the composition is osanetant p-toluenesulfonic acid Form 1. In certain embodiments, provided is a composition comprising osanetant, wherein at least about 99% of the osanetant present in the composition is osanetant p-toluenesulfonic acid Form 1. In certain embodiments, provided is a composition comprising osanetant, wherein at least about 99.5% of the osanetant present in the composition is osanetant p-toluenesulfonic acid Form 1. In certain embodiments, provided is a composition comprising osanetant, wherein at least about 99.9% of the osanetant present in the composition is osanetant p-toluenesulfonic acid Form 1. In certain embodiments, provided is a composition comprising osanetant, wherein at least about 99.99% of the osanetant present in the composition is osanetant p-toluenesulfonic acid Form 1.

In certain embodiments, provided is a composition comprising osanetant, wherein at least about 85% of the osanetant present in the composition is osanetant p-toluenesulfonic acid Form 6. In certain embodiments, provided is a composition comprising osanetant, wherein at least about 90% of the osanetant present in the composition is osanetant p-toluenesulfonic acid Form 6. In certain embodiments, provided is a composition comprising osanetant, wherein at least about 95% of the osanetant present in the composition is osanetant p-toluenesulfonic acid Form 6. In certain embodiments, provided is a composition comprising osanetant, wherein at least about 97% of the osanetant present in the composition is osanetant p-toluenesulfonic acid Form 6. In certain embodiments, provided is a composition comprising osanetant, wherein at least about 98% of the osanetant present in the composition is osanetant p-toluenesulfonic acid Form 6. In certain embodiments, provided is a composition comprising osanetant, wherein at least about 99% of the osanetant present in the composition is osanetant p-toluenesulfonic acid Form 6. In certain embodiments, provided is a composition comprising osanetant, wherein at least about 99.5% of the osanetant present in the composition is osanetant p-toluenesulfonic acid Form 6. In certain embodiments, provided is a composition comprising osanetant, wherein at least about 99.9% of the osanetant present in the composition is osanetant p-toluenesulfonic acid Form 6. In certain embodiments, provided is a composition comprising osanetant, wherein at least about 99.99% of the osanetant present in the composition is osanetant p-toluenesulfonic acid Form 6.

Also provided herein, in some embodiments, are pharmaceutical compositions that comprise a crystalline salt form of osanetant, and one or more pharmaceutically acceptable vehicles selected from carrier, adjuvants, and excipients.

Suitable pharmaceutically acceptable vehicles may include, for example, inert solid diluents and fillers, diluents, including sterile aqueous solutions and various organic solvents, permeation enhancers, solubilizers, and adjuvants. Such compositions are prepared in a manner well known in the pharmaceutical art. See, e.g., Remington's Pharmaceutical Sciences, Mace Publishing Co., Philadelphia. Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal, intravenous, subcutaneous, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

The pharmaceutical composition may be in the form of a sterile injectable aqueous or oleaginous suspension. This suspension may be formulated according to the known art using suitable dispersing or wetting agents and suspending agents. The sterile injectable preparation may also be sterile injectable solution or suspension in a non-toxic parentally acceptable vehicle, for example as a solution in 1,3-butanediol. Among the acceptable vehicles that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid can be useful in the preparation of injectables. Such solutions may be formulated as 0.01%-10% isotonic solutions, pH 5-7, with appropriate salts.

The crystalline salt forms of osanetant described herein may be administered parenterally in a sterile medium. Parenteral administration includes subcutaneous injections, intravenous, intramuscular, intrathecal injection or infusion techniques. The crystalline salt forms of osanetant described herein, depending on the vehicle and concentration used, can either be suspended or dissolved in the vehicle. Advantageously, adjuvants such as local anesthetics, preservatives and buffering agents can be dissolved in the vehicle. In many pharmaceutical compositions for parenteral administration the carrier comprises at least 90% by weight of the total composition. In some embodiments, the carrier for parenteral administration is chosen from propylene glycol, ethyl oleate, pyrrolidone, ethanol, and sesame oil.

A pharmaceutical composition, for example, for injection, may comprise a cyclodextrin. The cyclodextrin may be, for example, a hydroxypropyl cyclodextrin or a sulfobutylether cyclodextrin. The cyclodextrin may be, for example, an α-cyclodextrin, a β-cyclodextrin, or a γ-cyclodextrin.

A compound described herein may also be administered via microspheres, liposomes, other microparticulate delivery systems or sustained release formulations placed in certain tissues including blood. Suitable examples of sustained release carriers include semi-permeable polymer matrices in the form of shared articles, e.g., suppositories or microcapsules. Examples can be found, e.g., in Remington's Pharmaceutical Sciences, 18th edition, Gennaro, A. R., Lippincott Williams & Wilkins; 20th edition (Dec. 15, 2000) ISBN 0-912734-043 and Pharmaceutical Dosage Forms and Drug Delivery Systems; Ansel, N. C. et al. 7th Edition ISBN 0-683305-72-7, the entire disclosures of which are herein incorporated by reference.

Oral administration may be another route for administration of the crystalline salt forms of osanetant described herein. Administration may be via, for example, capsule or enteric coated tablets. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the crystalline salt forms of osanetant described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein. When referring to these preformulation compositions as homogeneous, the active ingredient may be dispersed evenly throughout the composition so that the composition may be readily subdivided into equally effective unit dosage forms such as tablets, pills and capsules.

The tablets or pills of the crystalline salt forms of osanetant described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The crystalline salt forms of osanetant described herein can be incorporated into oral liquid preparations such as aqueous or oily suspensions, solutions, emulsions, syrups, or elixirs, for example. Furthermore, pharmaceutical compositions containing the crystalline salt forms of osanetant described herein can be presented as a dry product for constitution with water or other suitable vehicle before use. Such liquid preparations can contain conventional additives, such as suspending agents (e.g., sorbitol syrup, methyl cellulose, glucose/sugar, syrup, gelatin, hydroxyethyl cellulose, carboxymethyl cellulose, aluminum stearate gel, and hydrogenated edible fats), emulsifying agents (e.g., lecithin, sorbitan monooleate, or acacia), non-aqueous vehicles, which can include edible oils (e.g., almond oil, fractionated coconut oil, silyl esters, propylene glycol and ethyl alcohol), and preservatives (e.g., methyl or propyl p-hydroxybenzoate and sorbic acid).

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, orally or nasally, from devices that deliver the formulation in an appropriate manner.

Buccal administration, where the pharmaceutical composition is placed between the gum and check and diffuses through the oral mucosa, may be another route for administration of the crystalline salt forms of osanetant described herein. The forms in which the pharmaceutical compositions described herein may be incorporated for buccal administration include, for example, quick-dissolving tablets, buccal mucoadhesive tablets, lozenges, powders, sprays, mucoadhesive buccal patches and films, ointments, gels, or liquid suspensions. Formulations for the pharmaceutical compositions for buccal administration that include at least one crystalline salt form described herein may also include mucoadhesive agents, to maintain prolonged contact of the formulation with the oral mucus membrane, penetration enhancers, to improve drug permeation across the oral mucus membrane, enzyme inhibitors, to protect the active ingredient from enzymatic degradation, and solubility modifiers. The active ingredient is also usually diluted by an excipient.

Some examples of suitable mucoadhesive agents include agarose, chitosan, trimethylated chitosan, chitosan-EDTA, gelatin, hyaluronic acid, guar gum, hakea gum, xanthan gum, gellan gum, carrageenan, pectin, sodium alginate, cellulose derivatives, CMC, thiolated CMC, sodium CMC, HEC, HPC. HPMC, MC, poly (acrylic acid)-based polymers, CP, PC, PAA, copolymers of acrylic acid and PEG, PVA, PVP, CP, aminodextran, dimethylaminoethyl-dextran, hydroxyethyl starch, poly (ethylene oxide), scleroglucan, cyanoacrylate, hydroxylated methacrylate, and poly (methacrylic acid). Some examples of suitable penetration enhancers include sodium lauryl sulfate, cetyl pyridinium chloride, Poloxamer, Brij. Span, Myrj. Tween, sodium glycocholate, sodium tauro deoxycholate, sodium tauro cholate, oleic acid, caprylic acid, lauric acid, lyso phosphatidyl choline, phosphatidyl choline, α-, β-, and γ-cyclodextrin, methylated β-cyclodextrin, EDTA, citric acid, sodium salicylate, methoxy salicylate, chitosan, trimethyl chitosan, poly-L-arginine, and L-lysine. Some examples of suitable enzyme inhibitors include aprotinin, bestatin, and puromycin.

### Combination Therapy

A crystalline salt form of osanetant as described herein may be administered in combination with one or more additional active agents. When used in combination with one or more additional active agents, a crystalline salt form of osanetant as described herein may be administered prior to, concurrently with, or following administration of the additional active agents. As such, the one or more additional active agents can be administered to the patient in a separate dose or dosage form, before, after, or concurrently with the crystalline salt form of osanetant as described herein, or in a combination therapy therewith. The administration can be by the same route or by different routes. Any additional active agent or treatment schedule will depend on both the disease or disorder being treated and the patient.

Thus, for the methods described herein which include treating one or more vasomotor symptoms, including, but not limited to, in a patient for which hormone therapy is contraindicated, such as wherein the patient will be undergoing a hormone deprivation therapy, and/or medical or surgical procedure that may cause VMS, the second active agent may be a SERM, SERD, GnRH or NSAA.

In certain embodiments, the methods described herein comprise administering, simultaneously or sequentially, an effective amount of a crystalline salt form of osanetant as described herein and one or more additional active agent(s), for a time period prior to, or concurrently with, the hormone deprivation therapy, and/or medical or surgical procedure. In certain embodiments of the administration of a combination therapy including a crystalline salt form of osanetant as described herein, the patient has cancer, has had cancer, or is at an increased risk for cancer.

As used herein, “selective estrogen-receptor modulators” or “SERMs” are a class of drug which have varied estrogenic and antiestrogenic effects on estrogen receptors, depending on the tissue in which the receptors are located. This allows for selective estrogen receptor modulation in certain tissue types by choosing the appropriate SERM.

As used herein, “gonadotropin-releasing hormone agonists and antagonists” or “GnRH agonists and antagonists” are classes of drugs which prevent the GnRH-mediated release of sex hormones. GnRH is a peptide hormone produced by GnRH neurons in the hypothalamus, responsible for the release of follicle-stimulating hormone and luteinizing hormone from the pituitary gland, beginning the hypothalamic-pituitary-gonadal axis synthesis and release of sex hormones.

As used herein, “nonsteroidal antiandrogens” or “NSAAs” are a class of drug that are antagonists of androgen receptors, blocking the action of testosterone and dihydrotestosterone in tissue.

As used herein, “selective estrogen receptor degraders” or “SERDs” are a class of drug that binds to the estrogen receptor (ER) and, in the process of doing so, causes the ER to be degraded and thus downregulated.

In some embodiments, the second active agent may be a selective estrogen receptor modulator (SERM), including, but not limited to, anordrin (+mifepristone (Zi Yun)), bazedoxifene (+conjugated estrogens (Duavee)), broparestrol (Acnestrol), clomifene (Clomid), cyclofenil (Sexovid), lasofoxifene (Fablyn), ormeloxifene (Centron, Novex, Novex-DS, Sevista), ospemifene (Osphena; deaminohydroxytoremifene), raloxifene (Evista), tamoxifen (Nolvadex), toremifene (Fareston; 4-chlorotamoxifen), acolbifene, afimoxifene (4-hydroxytamoxifen; metabolite of tamoxifen), elacestrant, enclomifene ((E)-clomifene), endoxifen (4-hydroxy-N-desmethyltamoxifen; metabolite of tamoxifen), zuclomifene ((Z)-clomifene), arzoxifene, brilanestrant, clomifenoxide (clomiphene N-oxide; metabolite of clomifene), droloxifene (3-hydroxytamoxifen), etacstil, fispemifene, GW-7604 (4-hydroxyetacstil; metabolite of etacstil), idoxifene (pyrrolidino-4-iodotamoxifen), levormeloxifene ((L)-ormeloxifene), miproxifene, nafoxidine, nitromifene (CI-628), NNC 45-0095, panomifene, pipendoxifene (ERA-923), trioxifene, or zindoxifene (D-16726). In some embodiments, the second active agent may be a gonadotropin-releasing hormone agonist, including, but not limited to, buserelin, deslorelin, fertirelin, gonadorelin, goserelin, histrelin, lecirelin, leuprorelin, nafarelin, peforelin, triptorelin, abarelix, cetrorelix, degarelix, ganirelix, elagolix, or relugolix. In some embodiments, the second active agent may be a nonsteroidal antiandrogen (NSAA), including, but not limited to, flutamide, nilutamide, bicalutamide, topilutamide, apalutamide, enzalutamide, darolutamide, cimetidine, proxalutamide, seviteronel, cioteronel, inocoterone acetate, or RU-58841.

In some embodiments, the second active agent may be a selective estrogen receptor degrader (SERD), including, but not limited to, fulvestrant, brilanestrant, elacestrant, SERD '859, GDC-9545, and AZD-9833.

In some embodiments, the second active agent may be a selective androgen receptor degrader (SARD), including, but not limited to, dimethylcurcumin.

In certain embodiments where the disease or disorder is phycological in nature, a crystalline salt form of osanetant as described herein can be administered with a second active agent or therapy to treat a psychiatric disorder.

One method of psychotherapy specifically contemplated is the use of virtual reality (VR) exposure therapy to treat a psychiatric disorder using the combination therapy protocol of the disclosure. VR therapy to treat certain conditions such as PTSD in, for example, Vietnam veterans (Rothbaum et al. 30 (1999) J. Trauma Stress 12 (2): 263-71) or rape victims (Rothbaum et al. (2001) J. Trauma Stress 14 (2): 283-93), one embodiment of the present disclosure specifically contemplates the use of such VR exposure psychotherapy in combination with a compound as described elsewhere herein to treat a psychological condition.

In certain embodiments, the crystalline salt form described herein is administered in combination with a second active agent such as an anti-depressant. In certain embodiments, the agent is sertraline, paroxetine, fluoxetine, citalopram, baclofen, modafinil, eszopiclone, hydrocortisone, varenicline, dexamethasone or combinations thereof.

In one embodiment, a crystalline salt form of osanetant as described herein is administered with a compound that inhibits kisspeptin/neurokinin B/dynorphin (KNDy) neurons. It is contemplated that any suitable compound that can either block KNDy neuronal activity or interfere with the ability of KNDy neurons to communicate with other neurons can be used. In non-limiting embodiments, the compounds may be kappa agonists, including but not restricted to those whose action is predominantly “peripheral” or outside of the confines the blood-brain barrier, so-called peripherally-restricted agents, such as peripherally restricted kappa agonists (PRKAs), neurokinin type 3 receptor (NK3R) antagonists, or neurokinin type 1 receptors (NK1R) antagonists, combinations thereof, and salts thereof, all of which can act in the hypothalamus to inhibit KNDy neuron activity (and potentially VMS related to thermal instability). In a preferred embodiment, the compound's action is predominantly “peripheral” or outside of the confines the blood-brain barrier, and the compounds are referred to as peripherally-restricted agents.

In one embodiment, a crystalline salt form of osanetant as described herein is administered with a kappa opioid agonist. In certain embodiments the kappa opioid receptor agonist is selected from alazocine, bremazocine, 8-carboxamidocyclazocine, cyclazocine, ketazocine, metazocine, pentazocine, phenazocine, 6′-guanidinonaltrindole (6′-GNTI), butorphan, butorphanol, cyclorphan, diprenorphine, etorphine, levallorphan, levomethorphan, levorphanol, morphine, nalbuphine, nalfurafine, nalmefene, nalodeine, nalorphine, norbuprenorphine, norbuprenorphine-3-glucuronide, oxilorphan, oxycodone, proxorphan, samidorphan, xorphanol, asimadoline, BRL-52537, eluxadoline, enadoline, GR-89696, ICI-204,448, ICI-199,441, LPK-26, MB-1C-OH, niravoline, N-MPPP, spiradoline, U-50,488, U-54,494A, U-69,593, CR665, difelikefalin (CR845), dynorphins (dynorphin A, dynorphin B, big dynorphin), collybolide, erinacine E, menthol, RB-64, salvinorin A, 2-methoxymethyl salvinorin B (and its ethoxymethyl and fluoroethoxymethyl homologues), apadoline, HS665, HZ-2, ibogaine, ketamine, noribogaine, tifluadom, and combinations thereof.

In a one embodiment, the kappa agonist is a peripherally restricted kappa agonist (PRKA), such as, but not limited to, one or more of ICI-204,448, Asimadoline, FE 200665, Fedotozine, any compound disclosed in U.S. Pat. No. 7,713,937 (including, but not limited to, Cara Therapeutics compound CR854), a peptide disclosed in U.S. Pat. No. 5,965,701, or a combination thereof or a pharmaceutically acceptable salt thereof. The effects of a peripherally restricted agent (be it a kappa agonist, NKB/NK3R antagonist, or a substance P/NKIR antagonist) is that it would have only limited access to the higher centers of the brain—areas controlling cognition, mood, affect, movement, and integrative sensory function (which are within the blood-brain barrier), but still have full access to parts of the brain that are outside of the blood brain barrier, including those areas of the hypothalamus that generate the VMS. This approach could avoid one or more side effects of centrally active agents (that cross the blood brain barrier), such as dysphoria and euphoria, mental confusion, drowsiness, diminished attentiveness, depression, and the like. Since efficacious kappa agonists produce compliance-limiting dysphoric responses when absorbed into brain, agents that do not efficiently penetrate the blood-brain barrier would produce the desired effect (e.g., reduction of hot-flash symptoms) without adverse cognitive or mood disturbance.

Also provided is a pharmaceutical composition comprising a crystalline salt form of osanetant as described herein and a second active agent.

### Dosing

The specific dose level of the one or more crystalline salt forms of osanetant as disclosed herein for any particular subject will depend upon a variety of factors including the activity of the specific crystalline salt form of osanetant employed, the age, body weight, general health, sex, diet, time of administration, route of administration, and rate of excretion, drug combination and the severity of the particular disease in the subject undergoing therapy. For example, a dosage may be expressed as a number of milligrams of osanetant administered, irrespective of the crystalline salt form, per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of osanetant administered per dose or per day. Daily dosage of osanetant may be between about 1 mg and 1,000 mg, between about 1 to 500 mg/day, between about 1 to 400 mg/day, between about 1 to 300 mg/day, between about 1 to 250 mg/day, between about 1 to 200 mg/day, between about 50 mg and 1,000 mg, between about 50 to 500 mg/day, between about 50 to 400 mg/day, between about 50 to 300 mg/day, between about 50 to 250 mg/day, between about 50 to 200 mg/day, between about 1 to 150 mg/day, between about 1 to 100 mg/day, between about 1 to 70 mg/day, between about 1 to 50 mg/day, between about 5 to 50 mg/day, between about 5 to 40 mg/day, between about 10 to 50 mg/day, between about 10 to 40 mg/day, between about 20 to 50 mg/day, between about 20 to 40 mg/day, or about 25 mg/day, or about 50 mg/day, or about 200 mg/day.

When administered orally, the total daily dosage for a human patient may be between 1 mg and 3000 mg, between 1 mg and 1,000 mg, between about 1-500 mg/day, between about 1-100 mg/day, between about 1-50 mg/day, between about 10-40 mg/day, or about 250 mg per day, or about 200 mg per day, or about 150 mg per day, or about 100 mg per day, or about 75 mg per day, or about 50 mg per day, or about 25 mg per day. In certain embodiments, when administered orally, the total daily dosage for a human patient is 50 mg/day, or 25 mg bis in die (twice a day, BID). In certain embodiments, when administered orally, the total daily dosage for a human patient is 100 mg/day, or 50 mg BID. In certain embodiments, when administered orally, the total daily dosage for a human patient is 200 mg/day, or 100 mg BID. In some embodiments, the total daily dosage for a human patient may be between 25 mg/day to 300 mg/day BID, 25 mg/day to 250 mg/day BID, 25 mg/day to 200 mg/day BID, 25 mg/day to 150 mg/day BID, 25 mg/day to 100 mg/day BID, 25 mg/day to 50 mg/day BID, 50 mg/day to 300 mg/day BID, 50 mg/day to 250 mg/day BID, 50 mg/day to 200 mg/day BID, 50 mg/day to 150 mg/day BID, 50 mg/day to 100 mg/day BID, 100 mg/day to 300 mg/day BID, 100 mg/day to 250 mg/day BID, 100 mg/day to 200 mg/day BID, 100 mg/day to 150 mg/day BID, 150 mg/day to 300 mg/day BID, 150 mg/day to 250 mg/day BID, 150 mg/day to 200 mg/day BID, 200 mg/day to 300 mg/day BID, 200 mg/day to 250 mg/day BID, or 250 mg/day to 300 mg/day BID.

The one or more crystalline salt forms of osanetant as disclosed herein, or a composition comprising the same, may be administered once, twice, three, or four times daily, using any suitable mode described above. Also, administration or treatment with a crystalline salt form of osanetant as disclosed herein may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

In a particular embodiment, the method comprises administering to the subject an initial daily dose of about 1 to 800 mg of a crystalline salt form of osanetant as disclosed herein and increasing the dose by increments until clinical efficacy is achieved. Increments of about 5, 10, 25, 50, or 100 mg can be used to increase the dose. The dosage can be increased daily, every other day, twice per week, or once per week.

### Processes

Also provided are processes for preparing osanetant, intermediates for use in the same, and crystalline salt forms of osanetant provided therefrom. In certain embodiments, the composition of osanetant provided herein is substantially free (e.g., contains less than 2%, less than 1.9%, less than 1.8%, less than 1.7%, less than 1.6%, less than 1.5%, less than 1.4%, less than 1.3%, less than 1.2%, less than 1.1%, less than 1.0%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, less than 0.01%, or less than 0%) of one or more of:
(i) osanetant hydrochloride;
(ii) (S)-N-(1-(3-(1-benzoyl-3-(3,4-dichlorophenyl)piperidin-3-yl)propyl)-4-phenylpiperidin-4-yl) acetamide, having the structure:

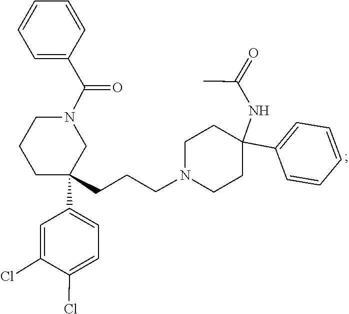 [SMILES: CC(=O)NC1(c2ccccc2)CCN(CCC[C@@]2(c3ccc(Cl)c(Cl)c3)CCCN(C(=O)c3ccccc3)C2)CC1]

(iii) (S)-(3-(3,4-dichloro-phenyl)-3-[3-(4-phenyl-piperidin-1-yl)-propyl]-piperidin-1-yl)-phenyl-methanone hydrochloride, having the structure:

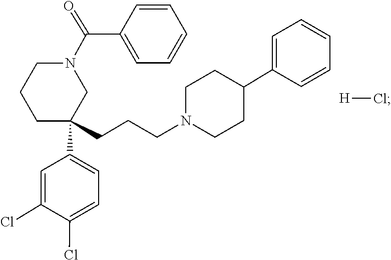

(iv) N-(4-phenylpiperidin-4-yl) acetamide, having the structure:

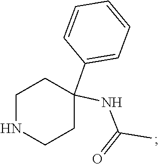 [SMILES: CC(=O)NC1(c2ccccc2)CCNCC1]

(v) (S)-{3-(3,4-dichloro-phenyl)-3-[3-(4-phenyl-piperidin-1-yl)-propyl]-piperidin-1-yl}-phenyl-methanone hydrochloride, having the structure:

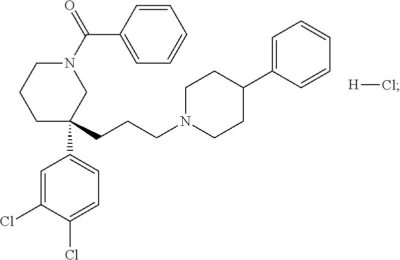

(vi) {3-(3,4-dichloro-phenyl)-3-[3-(4-phenyl-3,6-dihydro-2H-pyridin-1-yl)-propyl]-piperidin-1-yl}-phenyl-methanone, having the structure:

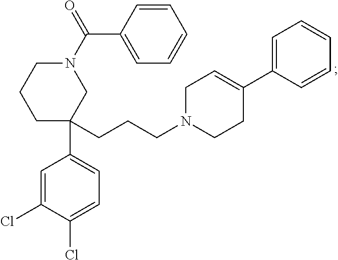 [SMILES: O=C(c1ccccc1)N1CCCC(CCCN2CC=C(c3ccccc3)CC2)(c2ccc(Cl)c(Cl)c2)C1]

or
(vii) a compound of formula D:

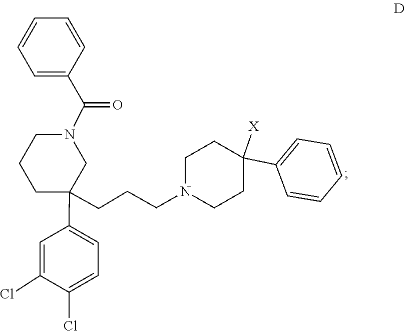

wherein X is other than —NHCH₃.

In certain embodiments, provided is a composition comprising osanetant, wherein the composition comprises at least one of the following:
(i) (R)-{3-(3,4-dichloro-phenyl)-3-[3-(4-methylamino-4-phenyl-piperidin-1-yl)-propyl]-piperidin-1-yl}-phenyl-methanone, having the structure:

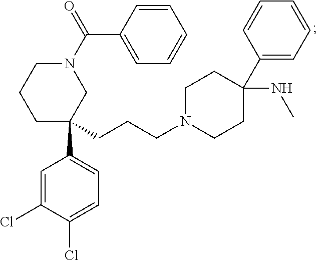 [SMILES: CNC1(c2ccccc2)CCN(CCC[C@]2(c3ccc(Cl)c(Cl)c3)CCCN(C(=O)c3ccccc3)C2)CC1]

(ii) benzenesulfonic acid-N-[1-{3- [(3R)-1-benzoyl-3-(3,4-dichlorophenyl)piperidin-3-yl] propyl}-4-([1,1′-biphenyl]-4-yl)piperidin-4-yl]-N-methylacetamide (1/1), having the structure:

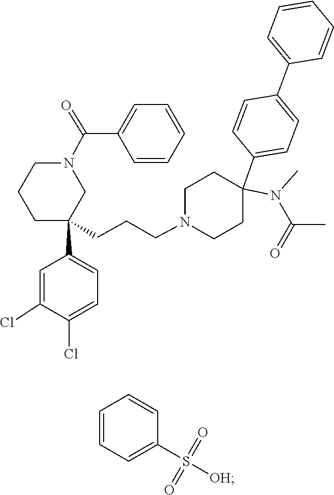 [SMILES: CC(=O)N(C)C1(c2ccc(-c3ccccc3)cc2)CCN(CCC[C@]2(c3ccc(Cl)c(Cl)c3)CCCN(C(=O)c3ccccc3)C2)CC1 ; O=S(=O)(O)c1ccccc1]

(iii) (S)-{3-(3,4-dichloro-phenyl)-3-[3-(4-phenyl-piperidin-1-yl)-propyl]-piperidin-1-yl}-phenyl-methanone hydrochloride, having the structure:

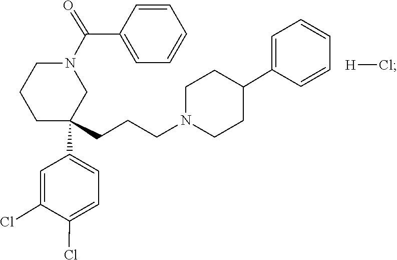

or
(iv) {3-(3,4-dichloro-phenyl)-3-[3-(4-phenyl-3,6-dihydro-2H-pyridin-1-yl)-propyl]-piperidin-1-yl}-phenyl-methanone, having the structure:

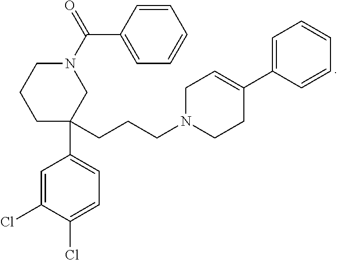 [SMILES: O=C(c1ccccc1)N1CCCC(CCCN2CC=C(c3ccccc3)CC2)(c2ccc(Cl)c(Cl)c2)C1]

In certain embodiments, the composition comprises not more than 0.1% of any one of the compounds of steps (i)-(iv).

In certain embodiments, the composition comprises not more than 1.50% of the sum of the compounds of steps (i)-(iv).

In certain embodiments, provided is a composition comprising osanetant, wherein the composition comprises (R)-{3-(3,4-dichloro-phenyl)-3-[3-(4-methylamino-4-phenyl-piperidin-1-yl)-propyl]-piperidin-1-yl}-phenyl-methanone, having the structure:

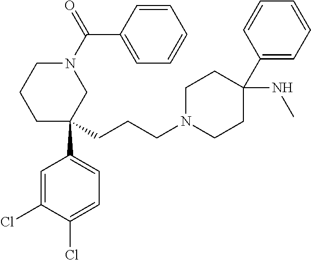 [SMILES: CNC1(c2ccccc2)CCN(CCC[C@]2(c3ccc(Cl)c(Cl)c3)CCCN(C(=O)c3ccccc3)C2)CC1]

in an amount not exceeding 0.2%.

In certain embodiments, the composition comprises not more than 0.1% of (R) {3-(3,4-dichloro-phenyl)-3-[3-(4-methylamino-4-phenyl-piperidin-1-yl)-propyl]-piperidin-1-yl}-phenyl-methanone.

In certain embodiments, provided is a process for preparing osanetant, or a salt or hydrate thereof, comprising contacting a compound of formula A, or a salt thereof:

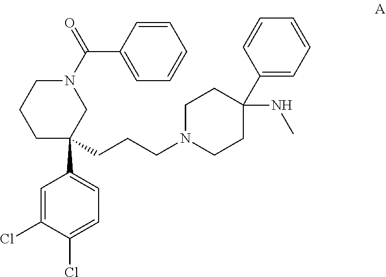 [SMILES: CNC1(c2ccccc2)CCN(CCC[C@]2(c3ccc(Cl)c(Cl)c3)CCCN(C(=O)c3ccccc3)C2)CC1]

with an acetylating agent under conditions sufficient to provide osanetant, or a salt thereof.

In certain embodiments, provided is a process for preparing osanetant, or salt thereof, comprising:
(i) contacting a compound of formula B, or salt thereof, with a compound of formula C, or salt or hydrate thereof:

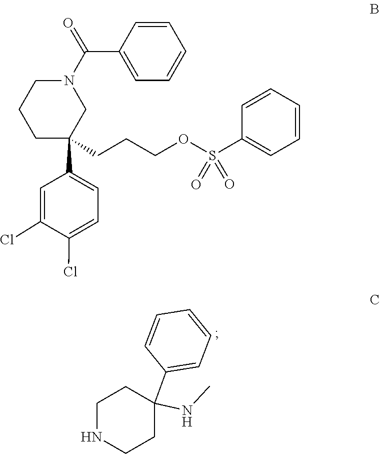 [SMILES: O=C(c1ccccc1)N1CCC[C@](CCCOS(=O)(=O)c2ccccc2)(c2ccc(Cl)c(Cl)c2)C1 ; CNC1(c2ccccc2)CCNCC1]

under conditions sufficient to provide a compound of formula A, or salt thereof:

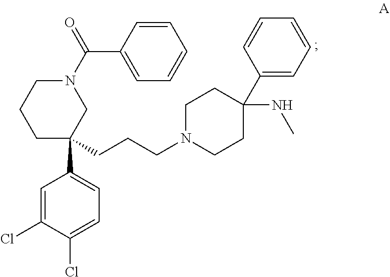 [SMILES: CNC1(c2ccccc2)CCN(CCC[C@]2(c3ccc(Cl)c(Cl)c3)CCCN(C(=O)c3ccccc3)C2)CC1]

and
(ii) contacting a compound of formula A, or salt thereof, with an acylating agent (e.g., an acyl halide, acetic anhydride, etc.), under conditions sufficient to provide osanetant, or salt thereof.

In certain embodiments, the acetylating agent is acetic anhydride.

In certain embodiments, the process further comprises contacting the osanetant, or a salt thereof, with a base to provide osanetant as a free base. In certain embodiments, the base is sodium hydroxide. In certain embodiments, the process further comprises isolating osanetant as a free base.

In certain embodiments, provided is a process for preparing a compound of formula A, or salt thereof:

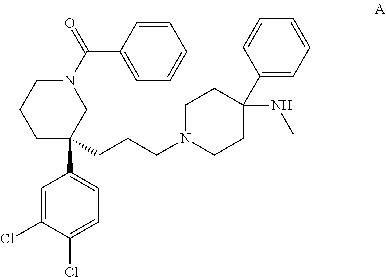 [SMILES: CNC1(c2ccccc2)CCN(CCC[C@]2(c3ccc(Cl)c(Cl)c3)CCCN(C(=O)c3ccccc3)C2)CC1]

comprising contacting a compound of formula B, or salt thereof, with a compound of formula C, or salt or hydrate thereof:

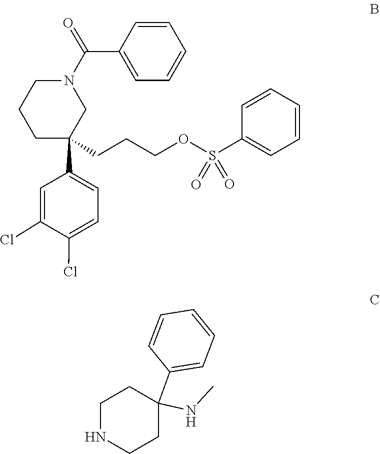 [SMILES: O=C(c1ccccc1)N1CCC[C@](CCCOS(=O)(=O)c2ccccc2)(c2ccc(Cl)c(Cl)c2)C1 ; CNC1(c2ccccc2)CCNCC1]

under conditions sufficient to provide of formula A, or salt thereof.

In certain embodiments, provided is a compound of formula A, or salt or hydrate thereof:

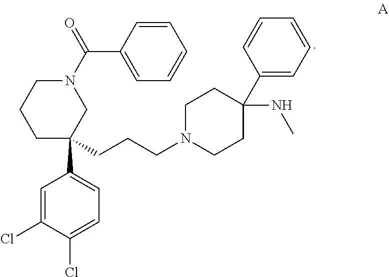 [SMILES: CNC1(c2ccccc2)CCN(CCC[C@]2(c3ccc(Cl)c(Cl)c3)CCCN(C(=O)c3ccccc3)C2)CC1]

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Synthesis of Osanetant

(R)-N-(1-(3-(1-benzoyl-3-(3,4-dichlorophenyl)piperidin-3-yl)propyl)-4-phenylpiperidin-4-yl)-N-methylacetamide naphthalene-2-sulfonic acid (osanetant) was prepared via the following method.

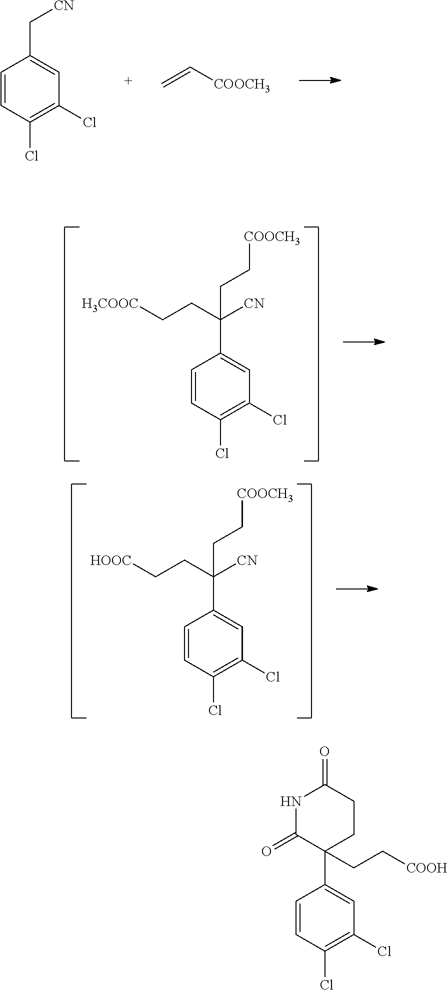

Step 1-Synthesis of 3-(3-(3,4-dichlorophenyl)-2,6-dioxopiperidin-3-yl) Propanoic Acid

To a glass-lined reactor containing a refluxing and stirring solution of about 18.7 kg of 3,4-dichlorophenylacetonitrile and 0.2 kg TRITON B in about 20 L of THF, was progressively added about 19 kg of methyl acrylate. The reaction progress was monitored by TLC, and upon completion, about 80 L of acetic acid were added, and the THF and volatile by-products were removed by distillation, until the temperature reached 115° C. In a separate vessel, about 2.5 kg of concentrated sulfuric acid and 5 kg of water were dissolved in about 20 L of acetic acid. The resulting solution was then added to the reaction mixture, which was then maintained under reflux for at least 30 minutes. The volatile by-products were again distilled until the temperate reached 110° C., and the reaction mixture was maintained at this temperature for at least 2 hours. The reaction mixture was then cooled to room temperature and the resulting product was collected by filtration. The filter cake was then washed with about 20 L of acetic acid and about 100 L of tert-butyl methyl ether, and then dried in a vacuum oven at about 70° C., to provide 3-(3-(3,4-dichlorophenyl)-2,6-dioxopiperidin-3-yl) propanoic acid.

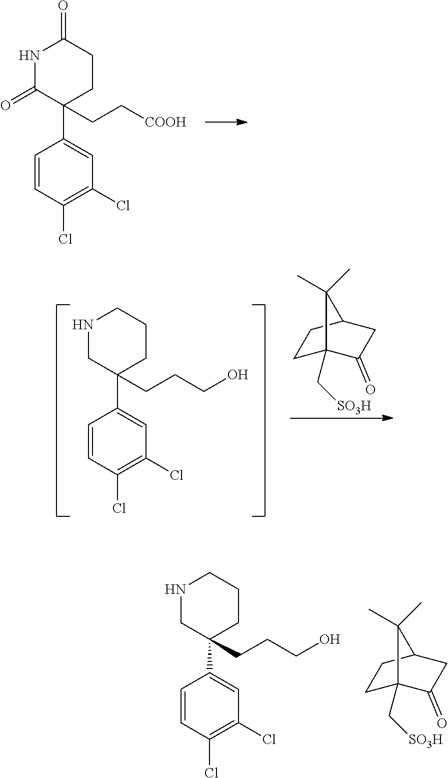

Step 2-Synthesis of(S)-3-(3-(3,4-dichlorophenyl)piperidin-3-yl) propan-1-ol(S)-camphorsulfonic Acid

To a stainless steel reactor under nitrogen was added about 12.5 kg of 3-(3-(3,4-dichlorophenyl)-2,6-dioxopiperidin-3-yl) propanoic acid and about 37 L of dry THF. To the resulting mixture was then added about 136 kg of 1M borane-tetrahydrofuran in THF in three parts: about 12 kg added at about 0° C., then about 40 kg added at 20-50° C., and finally about 84 kg added at reflux. The reaction mixture was maintained under reflux for at least 2 hours. Reaction progress was monitored by TLC, and upon completion, excess borane-tetrahydrofuran was quenched with about 22 L of methanol. Approximately 160 L of solvent was removed by distillation, and then a sulfuric acid solution of about 5 L of concentrated sulfuric acid in about 36 L of water was added, and the resulting mixture was heated to a boil until the solvent was completely evaporated. Reflux was then continued for at least 1 hour. The resulting mixture was then cooled to about 10° C. and a 30% sodium hydroxide solution was added to obtain a pH of 14. The product was extracted with about 47 L of butan-1-ol, and the organic layer was washed twice with about 20 kg of a sodium chloride solution of 2 kg of sodium chloride in 18 kg of water. Approximately 36 L of solvent were removed by distillation, followed by the addition of 36 L of propan-2-ol. The resulting solution was brought to reflux, and then a solution of about 9.6 kg of(S)-camphorsulfonic acid in about 75 L of propan-2-ol was added. The solution was then cooled below 20° C., and the crude product was collected by filtration. The filter cake was then washed with propan-2-ol and purified by recrystallization. 25 kg of the crude product was then suspended in about 325 L of propan-2-ol in a glass-lined reactor under nitrogen. The crude product was dissolved under reflux, and the solution was then slowly cooled to about 20° C. Pure(S)-3-(3-(3,4-dichlorophenyl)piperidin-3-yl)propan-1-ol(S)-camphorsulfonic acid then precipitated from the solution and was collected by filtration. The product was then washed with propan-2-ol and dried in a vacuum oven at about 50° C.

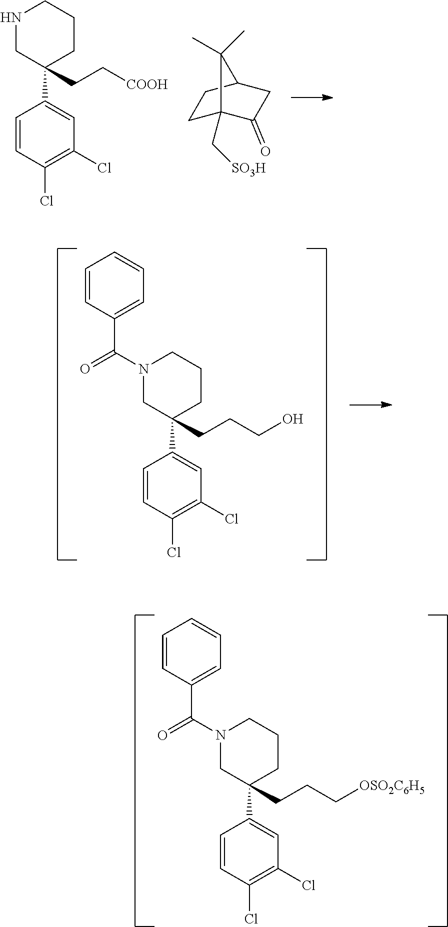

Step 3-Synthesis of(S)-3-(1-benzoyl-3-(3,4-dichlorophenyl)piperidin-3-yl)propyl benzenesulfonate

To a glass-lined reactor was added about 32 L of water, about 8 L of 30% sodium hydroxide, about 75 L of toluene, and about 17.2 kg of(S)-3-(3-(3,4-dichlorophenyl)piperidin-3-yl) propan-1-ol(S)-camphorsulfonic acid, and the resulting mixture was stirred for at least 30 minutes. Then 4.7 kg of benzoyl chloride was added between about 20-40° C. Reaction progress was monitored with TLC and HPLC, and then the aqueous layer was discarded and the remaining organic layer was washed successively with about 20 L of water, 10 L of water, 0.15 L of concentrated sulfuric acid, and then 20 L of water. To the remaining organic layer was then added a solution of about 12 kg of sodium hydroxide in about 12 kg of water, about 0.4 kg of benzyltriethyl-ammonium chloride, and then, under vigorous stirring at between 35-45° C., about 11.7 kg of benzenesulfonyl chloride. Reaction progress was monitored with TLC and HPLC, and additional benzenesulfonyl chloride was added as necessary. The mixture was stirred until the benzenesulfonyl chloride conversion was completed, then about 75 L of water was added. The aqueous layer was then discarded and the remaining organic layer was washed with about 25 L of water. The washing process was repeated until pH=8. The resulting toluenic solution was dried azeotropically under vacuum and then used directly in a subsequent step.

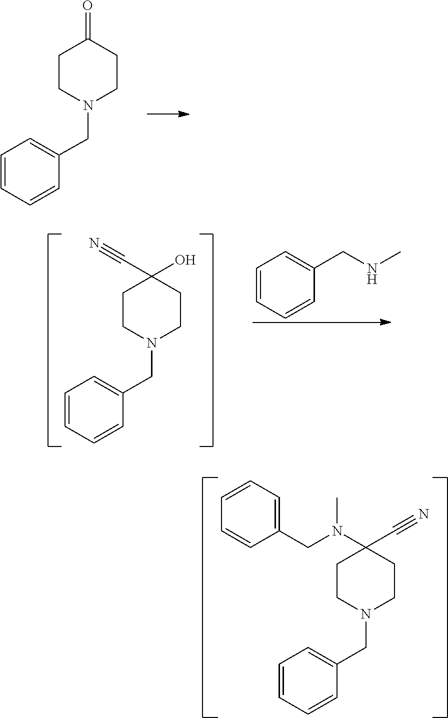

Step 4-Synthesis of 1-benzyl-4-(benzyl (methyl) amino)piperidine-4-carbonitrile

In a glass-lined reactor under nitrogen was added 7.3 kg of potassium cyanide, about 8.6 kg of water, and 56.5 kg of toluene, and the resulting solution was stirred. The solution was then cooled to between 15-20° C., and an aqueous solution of about 19.2 kg of 1-benzyl-4-piperidone and 10.2 kg of concentrated hydrochloric acid in about 10.4 kg of water was added over 3 hours. Reaction progress was monitored by NMR, and, upon completion, the aqueous layer was discarded while the reaction mixture was at a maintained temperature of 45° C. The remaining stirred organic phase was the cooled to 20° C., and about 24.8 kg of magnesium sulfate was added. The resulting mixture was then heated to 45° C., and about 13.3 kg of N-benzylmethylamine was added. Reaction progress was again monitored by NMR. Upon completion, about 65 kg of water was added at 40° C. The resulting mixture was stirred, then the aqueous layer was discarded. The remaining organic layer was cooled to 15° C. and then washed once with about 1.5 kg of acetic acid and 30 kg of water, then twice with about 26 kg of water, then once with about 21 kg of a 1.5% sodium hydroxide solution, and finally twice with about 20 kg of water, until the washing were at a pH of less than 9. The resulting toluenic solution was then azeotropically dried and then used directly in the next step.

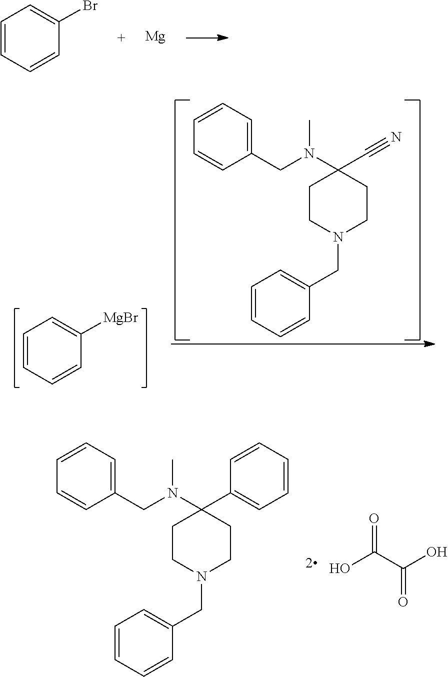

Step 5-Synthesis of N,1-dibenzyl-N-methyl-4-phenylpiperidin-4-amine Dioxalic Acid

In a glass-lined reactor under nitrogen was added about 2.2 kg of magnesium and about 7 kg of tert-butyl methyl ether dried on molecular sieves, and the resulting suspension was stirred. The suspension was brought to reflux, and then a mixture of about 14.1 kg of bromobenzene, 17.2 kg of toluene, and 16.3 kg of tert-butyl methyl ether dried on molecular sieves was added, and the resulting mixture was refluxed for at least 1 hour. The mixture was then cooled to between 10-15° C., and then about 9.5 kg of 1-benzyl-4-(benzyl (methyl) amino)piperidine-4-carbonitrile was added. Reaction progress was monitored by TLC until completion. In a separate vessel, about 45 kg of water and 3.4 kg of a 30% solution of hydrogen peroxide in water were stirred. The solution was then cooled to between 0-10° C., and the completed reaction mixture was added. The aqueous layer was then discarded, and the remaining organic layer, at a maintained temperature of 15° C., was washed once with about 22 kg of water and 2.7 kg of acetic acid, and once with about 15 kg of water. Then about 36 kg of water and 9 kg of concentrated hydrochloric acid were added, and the resulting mixture was stirred for at least 3 hours, until no unreacted starting material remained. About 12.6 kg of a 30% sodium hydroxide solution was added, and then the aqueous layer was discarded. The remaining organic layer was then washed with about 15 kg of water, and the aqueous layer was again discarded. The product was extracted with about 22 kg of water and 6.6 kg of concentrated hydrochloric acid. The aqueous layer was then basified with 8.7 kg of a 30% sodium hydroxide solution, and then extracted with about 35.6 kg of butan-1-ol. The aqueous layer was then discarded, and the remaining organic phase was washed with about 22 kg of water, until the washing were at a pH of less than 11. The resulting organic solution was azeotropically dried at 65° C., and then about 5.3 kg of oxalic acid in 67.8 kg of ethanol was added. The resulting mixture was then cooled to 20° C. overnight, then filtered and washed with about 17.2 kg of ethanol. The product was dried in a vacuum oven at about 60° C. to provide N, 1-dibenzyl-N-methyl-4-phenylpiperidin-4-amine dioxalic acid.

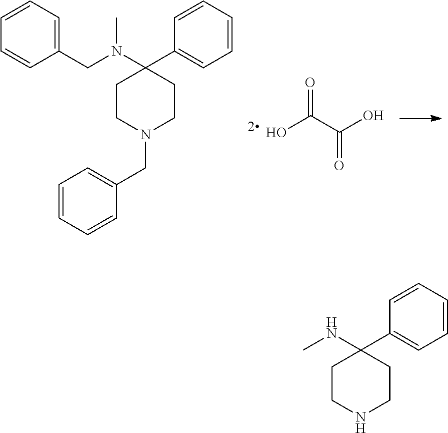 [SMILES: CN(Cc1ccccc1)C1(c2ccccc2)CCN(Cc2ccccc2)CC1 ; O=C(O)C(=O)O ; CNC1(c2ccccc2)CCNCC1]

Step 6-Synthesis of N-methyl-4-phenylpiperidin-4-amine

To an autoclave under nitrogen was added about 19 kg of N, 1-dibenzyl-N-methyl-4-phenylpiperidin-4-amine dioxalic acid and 120 kg of methanol. To this suspension was added about 1 kg of 5% palladium on charcoal in 20 kg of methanol. The resulting mixture was heated to about 40-45° C. under an atmosphere of hydrogen at about 3-7 PSI, until reaction completion as determined by TLC. Under a nitrogen atmosphere, about 30.5 kg of water was added to the reaction, and the resulting mixture was heated to about 60-65° C. The catalyst was then filtered off and washed with about 38 kg of water. Solvent was then distilled off to obtain a concentrated solution. About 86.4 kg of butan-1-ol was then added at about 93° C.+3° C. The resulting mixture was heated to reflux for at least 1 hour, then was cooled to 20° C.+3° C. The mixture was then filtered, and the filter cake was washed with a mixture of about 1.2 kg of water and 8.6 kg of butan-1-ol. The filter cake was then dried in a vacuum oven at about 60° C. to provide N-methyl-4-phenylpiperidin-4-amine.

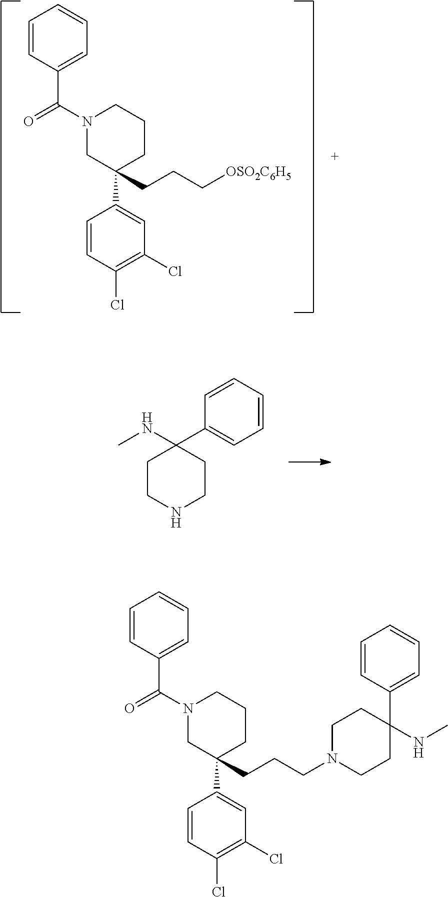

Step 7-Synthesis of (R)-(3-(3,4-dichlorophenyl)-3-(3-(4-(methylamino)-4-phenylpiperidin-1-yl)propyl)piperidin-1-yl) (Phenyl) Methanone

Into a glass-lined reactor was added about 15 kg of the toluenic product solution of(S)-3-(1 -benzoyl-3-(3,4-dichlorophenyl)piperidin-3-yl)propyl benzenesulfonate from step 3. The solution was stirred under vacuum, then heated to distill the toluene. About 74 kg of 4-methyl-2-pentanone was then added. The resulting mixture was then distilled to completely remove the toluene and 4-methyl-2-pentanone as an azeotropic mixture. In a second glass-lined reactor, about 9.4 kg of N-methyl-4-phenylpiperidin-4-amine was dissolved in 12.6 kg of water, under nitrogen. The pH of the resulting solution was adjusted to 14 with about 6.2 kg of potassium hydroxide and about 18.8 kg of water. The solution was extracted with about 30.4 kg of 4-methyl-2-pentanone. The aqueous layer was then re-extracted with about 11.3 kg of 4-methyl-2-pentanone, and the organic layers were combined. About 11.7 kg of a 50% potassium carbonate solution was then added. The resulting mixture was then heated to 85° C.±3° C., and the solution of about 45.8 kg of(S)-3-(1-benzoyl-3-(3,4-dichlorophenyl)piperidin-3yl)propyl benzenesulfonate was progressively added. The reaction conditions were maintained until completion, as determined by TLC and HPLC. The reaction mixture was then cooled to 20° C. and extracted with about 35.2 kg of a 14.5% acetic acid solution. The organic layer was then re-extracted with about 17.6 kg of a 14.5% acetic acid solution and the collected aqueous layers were combined and washed with about 18.1 kg of 4-methyl-2-pentanone. The organic layer was then isolated and discarded. The aqueous layer was then basified with about 17 kg of a 30% sodium hydroxide solution, and then extracted at 85-89° C. with about 84.3 kg of 4-methyl-2-pentanone. The organic layer was then washed three times with about 30.1 kg of water. The organic layer was then dried azeotropically and concentrated. The concentrated organic solution was crystallized at 70° C. for at least 1 hour, then cooled to about 0° C. The solids were filtered off and washed with about 7 kg of 4-methyl-2-pentanone, then dried in a vacuum oven at about 80° C. to provide (R)-(3-(3,4-dichlorophenyl)-3-(3-(4-(methylamino)-4-phenylpiperidin-1-yl)propyl)piperidin-1-yl) (phenyl) methanone.

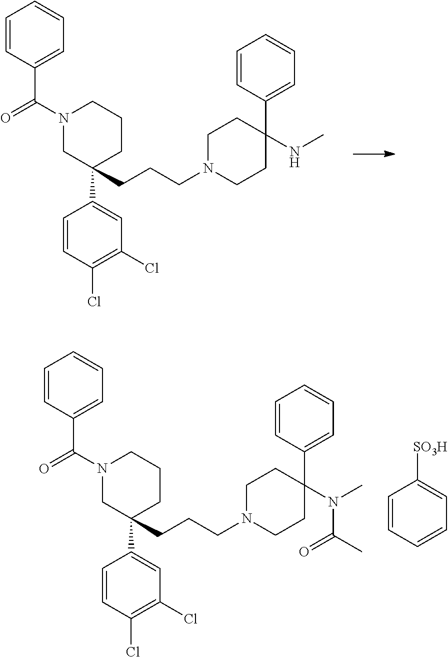

Step 8-Synthesis of (R)-N-(1-(3-(1-benzoyl-3-(3,4-dichlorophenyl)piperidin-3-yl)propyl)-4-phenylpiperidin-4-yl)-N-methylacetamide Benzenesulfonic Acid

To a glass-lined reactor was added about 12.5 kg of (R)-(3-(3,4-dichlorophenyl)-3-(3-(4 -(methylamino)-4-phenylpiperidin-1-yl)propyl)piperidin-1-yl) (phenyl) methanone and 70 kg of 4-methyl-2-pentanone, and the resulting suspension was stirred under nitrogen. To the suspension was then added about 4.5 kg of acetic anhydride at 65° C. The mixture was stirred at 65° C. for at least 2.5 hours, until reaction completion was determined by HPLC. The reaction mixture was cooled to 20° C., and then it was added at 65° C.+3° C. to a solution of about 10.1 kg of potassium carbonate in 25 kg of water. The resulting mixture was heated under reflux for at least 30 minutes, then the aqueous layer was isolated and discarded. The remaining organic layer was then washed twice with about 25 kg of water at 87° C.±2° C., then azeotropically dried and concentrated. The concentrated organic layer was then cooled to 25° C.±2° C., and about 3.6 kg of benzenesulfonic acid in about 9.2 kg of 4-methyl-2-pentanone was added. The mixture was kept at 25° C.±2° C. with stirring for at least 15 hours. Then the mixture was filtered and the filter cake was washed twice with about 10.5 kg of 4-methyl-2-pentanone. The resulting product was then dried on the filter with a flow of nitrogen at about 25° C. to provide (R)-N-(1-(3-(1-benzoyl-3-(3,4 -dichlorophenyl)piperidin-3-yl)propyl)-4-phenylpiperidin-4-yl)-N-methylacetamide benzenesulfonic acid.

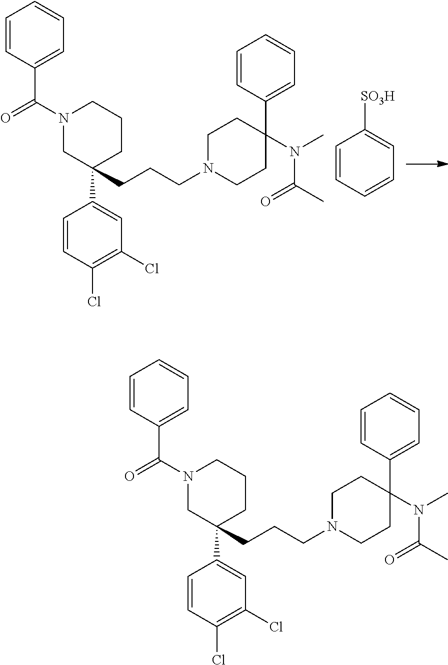

Step 9-Crystallization of (R)-N-(1-(3-(1-benzoyl-3-(3,4-dichlorophenyl)piperidin-3-yl)propyl)-4-phenylpiperidin-4-yl)-N-methylacetamide Form I

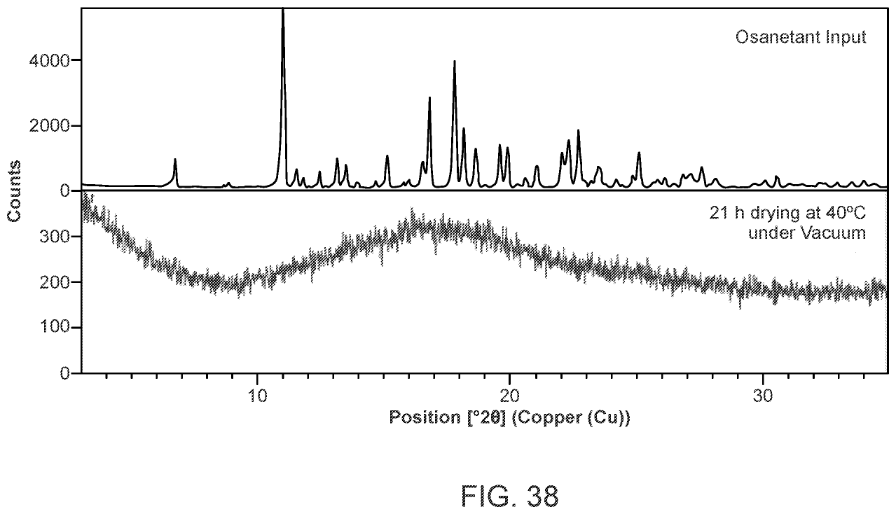

To a glass-lined reactor was added about 16.4 kg of (R)-N-(1-(3-(1-benzoyl-3-(3,4 -dichlorophenyl)piperidin-3-yl)propyl)-4-phenylpiperidin-4-yl)-N-methylacetamide benzenesulfonic acid, about 65.2 kg of methylene chloride, and 38 kg of water, and the resulting mixture was stirred under nitrogen. To the mixture was then added about 3 kg of a 30% sodium hydroxide solution at 20° C. The resulting mixture was then stirred for at least 15 minutes, then the organic layer was separated. The aqueous layer was then re-extracted with about 11 kg of methylene chloride, and the organic layers were combined. The combined organic layers were then washed twice with about 32.7 kg of water, then the methylene chloride was distilled. About 43 kg of ethanol was then added. The methylene chloride and ethanol were then distilled as an azeotropic mixture, until the methylene chloride was completely removed. About 32.2 kg of water was then added to the solution at about 70-75° C. The mixture was then cooled to about 20-25° C. and stirred for at least 15 hours. The mixture was then heated to 48° C.±2° C. for at least 3 hours, then cooled to 0° C.±2° C. for at least 15 hours. The mixture was then filtered and the collected solids washed with a mixture of about 6.8 kg of ethanol and 5.7 kg of water at 0° C.±2° C. The solids were then dried in a vacuum oven at about 80° C. to provide (R)-N-(1-(3-(1-benzoyl-3-(3,4-dichlorophenyl)piperidin-3-yl)propyl)-4-phenylpiperidin-4-yl)-N-methylacetamide (crystalline free base; top spectra of FIG. 38).

### Example 2

### Amorphous Lyophilized Osanetant

Amorphous lyophilized osanetant was prepared as follows. 1.52 g of osanetant was dissolved in 129 mL of 1,4-dioxane with gentle heating. The solution was split between 74 vials (ca. 20 mg per vial) and frozen, then lyophilized. After lyophilization, the solids were dried at 40° C. under vacuum for 21 h. After drying, the material from one of the vials was analyzed by XRPD to ensure that the material was amorphous and ¹H NMR to assess residual 1,4-dioxane content. XRPD analysis of the material after 21 h of drying at 40° C. under vacuum showed that the material was amorphous (FIG. 38, bottom spectra).

The osanetant used to prepare the amorphous lyophilized osanetant via the method described above was obtained according to Example 2. However, it should be appreciated that osanetant for use in the processes described herein may also be obtained from commercial sources as well as via alternative procedures, including those known in the art.

### Example 3

### Process for Preparing Crystalline Salt Forms of Osanetant

To approximately 20 mg of amorphous lyophilized osanetant was added 0.3 mL of solvent for liquid counterion experiments. 0.2 mL of solvent was employed for solid counterion experiments. The resulting mixture was stirred at 25° C.

To the stirring mixture was then added 1.05 mol eq. of the appropriate counterion. Where solid counterions were used, the vial which contained the counterion was washed in to the osanetant containing vial with 0.1 mL of the appropriate solvent. Additional solvent was added where necessary.

The experiments were thermally cycled as follows: heated from 25 to 40° C. at a rate of 0.1° C./min; 1 h at 40° C.; Cool to 5° C. at a rate of 0.1° C.; 1 h at 5° C.; 40° C. at a rate of 0.1° C./min; Cycle between 40 and 5° C.

After 2-3 days, anti-solvent addition was carried out on any clear solutions. 100 μL aliquots of the appropriate anti-solvent was added dropwise to the experiments, up to 1 mL total. The solvents and anti-solvents used in the formation of osanetant salts are shown in Table 1. The crystallization experiments were thermally cycled overnight, then slurries were isolated by centrifugation.

TABLE 1

| Number | Solvent | Anti-Solvent |
|---|---|---|
| 1 | 2-Propanol:Water 75:25% v/v | Water |
| 2 | Acetone | Heptane |
| 3 | Acetonitrile | Water |
| 4 | Ethanol | Heptane |
| 5 | Ethyl Acetate | Heptane |
| 6 | Tetrahydrofuran | Heptane |

XRPD analysis was carried out on any isolated solids or gums. The XRPD plate, and the isolated solids, were then dried at 40° C. for about 24 h, and XRPD analysis was repeated. The XRPD plate was then dried at 40° C./75% RH for about 24 h and XRPD analysis was again repeated. All other samples were allowed to evaporated at ambient temperature and pressure.

The following counterions were tested in the formation of osanetant salts via Method A: hydrochloric acid, 1,5-naphthalenedisulfonic acid, sulfuric acid, ethane-1,2-disulfonic acid, ethanesulfonic acid, p-toluenesulfonic acid, methanesulfonic acid, naphthalene-2-sulfonic acid, benzenesulfonic acid, maleic acid, phosphoric acid, glutamic acid, 1-hydroxy-2-napthoic acid, malonic acid, gentisic acid, (+)-L-tartaric acid, fumaric acid, citric acid, L-malic acid, hippuric acid, benzoic acid, succinic acid, adipic acid, and acetic acid. Results are discussed below and shown in the appended Figures.

### Hydrochloric Acid

The following observations were made using hydrochloric acid as the counterion in Method A. Clear solutions were observed on acid addition. White material was observed in ethyl acetate initially, however dissolution was then observed. Clear solutions were observed after thermal cycling for 2 days. After anti-solvent addition, slurries were observed in three of the experiments. Amorphous material was obtained from three of the solvent systems. The amorphous material did not crystallize on drying at 40° C. or 40° C./75% RH. Results are summarized in Table 2.

TABLE 2

| | | | Anti-Solvent Addition | | | Observation | XRPD | | |
|---|---|---|---|---|---|---|---|---|---|
| | Observation | | Anti- | Volume/ | | 3 Day | | | 40° C./ |
| Solvent | Initial | 2 Day | Solvent | mL | Observation | (total) | Wet | 40° C. | 75% RH |
| 2- | Clear | Clear | Water | 1 | Clear | Thin Slurry | | | |
| Propanol:Water | Solution | Solution | | | Solution | | | | |
| 75:25% v/v | | | | | | | | | |
| Acetone | Clear | Clear | Heptane | 0.6 | Slurry | Clear | N/A | N/A | N/A |
| | Solution | Solution | | | | Solution | | | |
| Acetonitrile | Clear | Clear | Water | 1 | Clear | Clear | N/A | N/A | N/A |
| | Solution | Solution | | | Solution | Solution | | | |
| Ethanol | Clear | Clear | Heptane | 1 | Clear | Clear | N/A | N/A | N/A |
| | Solution | Solution | | | Solution | Solution | | | |
| Ethyl Acetate | Clear | Clear | Heptane | 0.2 | Slurry | Gum | | | |
| | Solution | Solution | | | | | | | |
| Tetrahydrofuran | Clear | Clear | Heptane | 0.2 | Slurry | Gum | | | |
| | Solution | Solution | | | | | | | |

### Methanesulfonic acid

The following observations were made using methanesulfonic acid as the counterion in Method A. Clear solutions were observed on acid addition. An additional 200 μL of solvent was added to the ethyl acetate experiment as a thick slurry was observed. Clear solutions were observed in 4 of the systems after thermal cycling for 2 days. After anti-solvent addition, clear solutions remained in 3 of the 4 experiments which required anti-solvent addition. Crystalline material was observed from 4 of the solvent systems. Two XRPD patterns were obtained. After drying at 40° C./75% RH, crystallinity was lost. Results are summarized in Table 3.

TABLE 3

| | | | Anti-Solvent Addition | | | Observation | XRPD | | |
|---|---|---|---|---|---|---|---|---|---|
| 0 | Observation | | Anti- | Volume/ | | 3 Day | | | 40° C./ |
| Solvent | Initial | 2 Day | Solvent | mL | Observation | (total) | Wet | 40° C. | 75% RH |
| 2- | Clear | Clear | Water | 1 | Clear | Clear | N/A | N/A | |
| Propanol:Water | Solution | Solution | | | Solution | Solution | | | |
| 75:25% v/v | | | | | | | | | |
| Acetone | Clear | Clear | Heptane | 0.4 | Slurry | Slurry | Form 1 | Form 1 | Amorphous |
| | Solution | Solution | | | | | | | |
| Acetonitrile | Clear | Clear | Water | 1 | Clear | Clear | N/A | N/A | |
| | Solution | Solution | | | Solution | Solution | | | |
| Ethanol | Clear | Clear | Heptane | 1 | Clear | Slurry | Form 2 | Form 2 | Amorphous |
| | Solution | Solution | | | Solution | | | | |
| Ethyl Acetate | Clear | Slurry | N/A | N/A | N/A | Slurry | Form 1 | Form 1 | Amorphous |
| | Solution - | | | | | | | Tr 2 | |
| | Slurry | | | | | | | | |
| Tetrahydrofuran | Clear | Slurry | N/A | N/A | N/A | Slurry | Form 1 | Form 1 | Amorphous |
| | Solution | | | | | | | Tr 2 | |

Tr = Trace of

The osanetant methanesulfonic acid Form 1 solids consisted of needle-like crystals. The X-ray powder diffraction (XRPD) pattern for osanetant methanesulfonic acid Form 1 is shown in FIG. 1. Select peaks which can be used to characterize osanetant methanesulfonic acid Form 1 include one or more of 12.6°2θ±0.2°2θ, 14.5°2θ±0.2°2θ, 16.8°2θ±0.2°2θ, 18.0°2θ±0.2°2θ, 18.2°2θ±0.2°2θ, 18.6°2θ±0.2°2θ, or 22.5°2θ±0.2°2θ.

The osanetant methanesulfonic acid Form 2 solids consisted of aggregated crystals with an undefined morphology. The X-ray powder diffraction (XRPD) pattern for osanetant methanesulfonic acid Form 2 is shown in FIG. 2. Select peaks which can be used to characterize osanetant methanesulfonic acid Form 2 include one or more of 8.0°2θ±0.2°2θ, 11.3°2θ±0.2°2θ, 15.0°2θ±0.2°2θ, 16.1°2θ±0.2°2θ, 18.3°2θ±0.2°2θ, or 19.5°2θ±0.2°2θ.

Further data on osanetant methanesulfonic acid Form 1 and osanetant methanesulfonic acid Form 2 is shown in Table 4.

TABLE 4

| | | | Simultaneous TG/DSC | | |
|---|---|---|---|---|---|
| XRPD | ¹H NMR | | TG - Mass | | |
| Pattern | Solvent | Counterion | Losses | DSC - Thermal Events | PLM |
| Form 1 | 0.03 eq. Acetone and | 1.1 eq. | 1.7 wt. % up to | Broad Endothermic event | Aggregated |
| | residual Heptane | | 75° C. | peak at 153.3° C. | Needles |
| | | | 14.0 wt. % up | | |
| | | | to 270° C. | | |
| Form 2 | No | 1 eq. | IS | IS | Small |
| | | | | | Aggregated |

### Sulfuric Acid

The following observations were made using sulfuric acid as the counterion in Method A. Clear solutions were observed on acid addition. An additional 200 μL of solvent was added to the ethyl acetate experiment as a thick slurry was observed. Clear solutions were observed in all of the solvent systems after thermal cycling for 2 days. After anti-solvent addition, slurries were observed in five of the experiments, however only one remained a slurry after further thermal cycling. A unique XRPD pattern was observed from ethanol, which changed on drying at 40° C. There was a loss in crystallinity on drying at 40° C./75% RH. Amorphous material was obtained from four of the experiments. Results are summarized in Table 5.

TABLE 5

| | | | Anti-Solvent Addition | | | Observation | XRPD | | |
|---|---|---|---|---|---|---|---|---|---|
| | Observation | | Anti- | Volume/ | | 3 Day | | | 40° C./ |
| Solvent | Initial | 2 Day | Solvent | mL | Observation | (total) | Wet | 40° C. | 75% RH |
| 2- | Clear | Clear | Water | 0.8 | Slurry | Gum | Amorphous | Amorphous | Amorphous |
| Propanol:Water | Solution | Solution | | | | | | | |
| 75:25% v/v | | | | | | | | | |
| Acetone | Clear | Clear | Heptane | 0.2 | Slurry | Gum | Amorphous | Amorphous | Amorphous |
| | Solution | Solution | | | | | | | |
| Acetonitrile | Clear | Clear | Water | 1 | Clear | Clear | N/A | N/A | N/A |
| | Solution | Solution | | | Solution | Solution | | | |
| Ethanol | Clear | Clear | Heptane | 1 | Slurry | Slurry | Form 1 | Form 2 | Form 3* |
| | Solution | Solution | | | | | | | |
| Ethyl Acetate | Clear | Clear | Heptane | 0.2 | Slurry | Gum | Amorphous | Amorphous | Amorphous |
| | Solution | Solution | | | | | | | |
| Tetrahydrofuran | Clear | Clear | Heptane | 0.2 | Slurry | Gum | Amorphous | Amorphous | Amorphous |
| | Solution | Solution | | | | | | | |

*Poorly Crystalline

The X-ray powder diffraction (XRPD) pattern for osanetant sulfuric acid Form 1 is shown in FIG. 3. Select peaks which can be used to characterize osanetant sulfuric acid Form 1 include one or more of 6.3°2θ±0.2°2θ, 11.4°2θ±0.2°2θ, 12.7°2θ±0.2°2θ, 14.3°2θ±0.2°2θ, 17.0°2θ±0.2°2θ, 19.0°2θ±0.2°2θ, or 19.4°2θ±0.2°2θ.

The osanetant sulfuric acid Form 2 solids consisted of aggregated crystals with an undefined morphology. The X-ray powder diffraction (XRPD) pattern for osanetant sulfuric acid Form 2 is shown in FIG. 4. Select peaks which can be used to characterize osanetant sulfuric acid Form 2 include one or more of 6.3°2θ±0.2°2θ, 14.3°2θ±0.2°2θ, 15.4°2θ±0.2°2θ, 17.1°2θ±0.2°2θ, 19.8°2θ±0.2°2θ, or 22.6°2θ±0.2°2θ.

Further data on osanetant sulfuric acid Form 2 is shown in Table 6.

TABLE 6

| | | | Simultaneous TG/DSC | | |
|---|---|---|---|---|---|
| XRPD | ¹H NMR | | TG - Mass | DSC - Thermal | |
| Pattern | Solvent | Counterion | Losses | Events | PLM |
| Form 2 | 0.2 eq. Ethanol | Shifts in comparison | 1.2 wt. % up to | Endothermic event | Small |
| | | to FB | 150° C. | with peak at 151.6° C., | Aggregated |
| | | | 13.9 wt. % up | Endothermic event | |
| | | | to 250° C. | onset 156.6° C. (peak at | |
| | | | | 165.9° C.) | |

### Ethanesulfonic Acid

The following observations were made using ethanesulfonic acid as the counterion in Method A. Clear solutions were observed on acid addition in all of the systems. An additional 200 μL of solvent was added to the ethyl acetate experiment as a thick slurry was observed. After anti-solvent addition, slurries were observed in two of the experiments, however only one remained a slurry after further thermal cycling. One XRPD pattern was observed from ethyl acetate, which changed on drying at 40° C. On drying at 40° C./75% RH, crystallinity was lost. Amorphous material was obtained from acetone. Results are summarized in Table 7.

TABLE 7

| | | | Anti-Solvent Addition | | | Observation | XRPD | | |
|---|---|---|---|---|---|---|---|---|---|
| | Observation | | Anti- | Volume/ | | 3 Day | | | 40° C./ |
| Solvent | Initial | 2 Day | Solvent | mL | Observation | (total) | Wet | 40° C. | 75% RH |
| 2-Propanol:Water | Clear | Clear | Water | 1 | Clear | Clear | N/A | N/A | |
| 75:25 % v/v | Solution | Solution | | | Solution | Solution | | | |
| Acetone | Clear | Clear | Heptane | 0.4 | Slurry | Slurry | Amorphous | Amorphous | Amorphous |
| | Solution | Solution | | | | | | | |
| Acetonitrile | Clear | Clear | Water | 1 | Clear | Clear | N/A | N/A | |
| | Solution | Solution | | | Solution | Solution | | | |
| Ethanol | Clear | Clear | Heptane | 0.2 | Slurry | Clear | N/A | N/A | |
| | Solution | Solution | | | | Solution | | | |
| Ethyl Acetate | Clear | Slurry | N/A | N/A | N/A | Slurry | Form 1 | Form 2 | Amorphous |
| | Solution | | | | | | | | |
| Tetrahydrofuran | Clear | Slurry | N/A | N/A | N/A | Slurry - | N/A | N/A | |
| | Solution | | | | | Gel Like | | | |

The X-ray powder diffraction (XRPD) pattern for osanetant ethanesulfonic acid Form 1 is shown in FIG. 5. Select peaks which can be used to characterize osanetant ethanesulfonic acid Form 1 includes one or more of 9.6°2θ±0.2°2θ, 11.1°2θ±0.2°2θ, 15.5°2θ±0.2°2θ, 17.8°2θ±0.2°2θ, 18.2°2θ±0.2°2θ, or 18.4°2θ±0.2°2θ.

The osanetant ethanesulfonic acid Form 2 solids consisted of aggregated crystals with an undefined morphology. The X-ray powder diffraction (XRPD) pattern for osanetant ethanesulfonic acid Form 2 is shown in FIG. 6. Select peaks which can be used to characterize osanetant ethanesulfonic acid Form 2 includes one or more of 6.4°2θ±0.2°2θ, 10.0°2θ±0.2°2θ, 13.5°2θ±0.2°2θ, 15.2°2θ±0.2°2θ, 17.0°2θ±0.2°2θ, or 20.4°2θ±0.2°2θ.

Further data on osanetant ethanesulfonic acid Form 2 is shown in Table 8.

TABLE 8

| | | | Simultaneous TG/DSC | | |
|---|---|---|---|---|---|
| XRPD | ¹H NMR | | TG - Mass | DSC - Thermal | |
| Pattern | Solvent | Counterion | Losses | Events | PLM |
| Form 2 | Trace | 1.1 eq. | Insufficient Solids for Analysis | | Small aggregated |
| | Solvent | | | | crystals |

### Naphthalene-1,5-disulfonic Acid

The following observations were made using naphthalene-1,5-disulfonic acid as the counterion in Method A. Dissolution of the counterion was not observed in all systems on addition. Amorphous/poorly crystalline (PC) material was obtained from all of the experiments, except from ethyl acetate: heptane, where unreacted contention was obtained. No crystallization was observed after drying at 40° C. and 40° C./75% RH. Results are summarized in Table 9.

TABLE 9

| | | | Anti-Solvent Addition | | | Observation | XRPD | | |
|---|---|---|---|---|---|---|---|---|---|
| | Observation | | Anti- | Volume/ | | 4 Day | | | 40° C./ |
| Solvent | Initial | 3 Day | Solvent | mL | Observation | (total) | Wet | 40° C. | 75% RH |
| 2-Propanol:Water | Clear | Clear | Water | 1 | Slurry | Gum | Amorphous/ | Amorphous/ | Amorphous/ |
| 75:25 % v/v | Solution | Solution | | | | | PC | PC | PC |
| Acetone | Gum | Clear | Heptane | 1 | Clear | Brown | Amorphous/ | Amorphous/ | Amorphous/ |
| | | Solution + | | | Solution | Gum | PC | PC | PC |
| | | Orange | | | | | | | |
| | | Gum | | | | | | | |
| Acetonitrile | Clear | Clear | Water | 1 | Thin | Gum | Amorphous/ | Amorphous/ | Amorphous/ |
| | Solution + | Solution | | | Slurry | | PC | PC | PC |
| | Counterion | | | | | | | | |
| Ethanol | Clear | Clear | Heptane | 1 | Hazy | Gum | Amorphous/ | Amorphous/ | Amorphous/ |
| | Solution | Solution | | | | | PC | PC | PC |
| Ethyl Acetate | Clear | Clear | Heptane | 1 | Clear | Slurry | Counterion | Counterion | Amorphous/ |
| | Solution + | Solution + | | | Solution + | | | | PC |
| | Counterion | Solids | | | Solids | | | | |
| Tetrahydrofuran | Clear | Clear | Heptane | 1 | Clear | Slurry | Amorphous/ | Amorphous/ | Amorphous/ |
| | Solution + | Solution | | | Solution | | PC | PC | PC |
| | Counterion | | | | | | | | |

### Ethane-1,2-disulfonic Acid

The following observations were made using ethane-1,2-disulfonic acid as the counterion in Method A. After 3 days of thermal cycling, clear solutions were observed in all of the solvent systems. After anti-solvent addition and further thermal cycling, amorphous gums were recovered from four of the systems. A partially crystalline XRPD pattern was obtained from ethanol: heptane. The material lost crystallinity after drying at 40° C./75% RH. Results are summarized in Table 10.

TABLE 10

| | | | Anti-Solvent Addition | | | Observation | XRPD | | |
|---|---|---|---|---|---|---|---|---|---|
| | Observation | | Anti- | Volume/ | | 4 Day | | | 40° C./ |
| Solvent | Initial | 3 Day | Solvent | mL | Observation | (total) | Wet | 40° C. | 75% RH |
| 2-Propanol:Water | Clear | Clear | Water | 1 | Hazy | Gum | Amorphous | Amorphous | Amorphous |
| 75:25 % v/v | Solution | Solution | | | | | | | |
| Acetone | Gum | Clear | Heptane | 1 | Clear | Gum | Amorphous | Amorphous | Amorphous |
| | | Solution | | | Solution | | | | |
| Acetonitrile | Clear | Clear | Water | 1 | Hazy | Clear | N/A | N/A | N/A |
| | Solution + | Solution | | | | Solution | | | |
| | Counterion | | | | | | | | |
| Ethanol | Clear | Clear | Heptane | 1 | Hazy | Slurry | Partially | Partially | Amorphous |
| | Solution | Solution | | | | | Crystalline | Crystalline | |
| Ethyl Acetate | Clear | Clear | Heptane | 1 | Clear | Gum | Amorphous | Amorphous | Amorphous |
| | Solution + | Solution | | | Solution + | | | | |
| | Counterion | | | | Solids | | | | |
| Tetrahydrofuran | Clear | Clear | Heptane | 1 | Hazy | Gum | Amorphous | Amorphous | Amorphous |
| | Solution + | Solution | | | | | | | |
| | Counterion | | | | | | | | |

### p-Toluenesulfonic Acid

The following observations were made using p-toluenesulfonic acid as the counterion in Method A. After 3 days of thermal cycling, clear solutions were observed in four of the solvent systems, and slurries in two. After anti-solvent addition and further thermal cycling, slurries were observed. Crystalline material was observed from all of the solvent systems. Four XRPD patterns were obtained. After drying at 40° C., Patterns 1 and 2 were retained, and Pattern 3 converted to Pattern 4 on drying. After drying at 40° C./75% RH, Pattern 1 was retained, Pattern 2 converted to Pattern 1. Pattern 4 lost crystallinity. Patterns 1-4 were assigned Forms 1-4, respectively. Results are summarized in Table 11.

TABLE 11

| | | | Anti-Solvent Addition | | | Observation | XRPD | | |
|---|---|---|---|---|---|---|---|---|---|
| | Observation | | Anti- | Volume/ | | 4 Day | | | 40° C./ |
| Solvent | Initial | 3 Day | Solvent | mL | Observation | (total) | Wet | 40° C. | 75% RH |
| 2-Propanol:Water | Clear | Clear | Water | 0.5 | Slurry | Slurry | Form 1 | Form 1 | Form 1 |
| 75:25 % v/v | Solution | Solution | | | | | | | |
| Acetone | Clear | Clear | Heptane | 1 | Clear | Slurry | Form 1 | Form 1 | Form 1 |
| | Solution | Solution | | | Solution | | | | |
| Acetonitrile | Clear | Clear | Water | 1 | Slurry | Slurry | Form 1 | Form 1 | Form 1 |
| | Solution | Solution | | | | | | | |
| Ethanol | Clear | Clear | Heptane | 1 | Clear | Slurry | Form 2 | Form 2 | Form 1 |
| | Solution | Solution | | | Solution | | | | |
| Ethyl Acetate | Clear | Thick | Heptane | N/A | N/A | Slurry | Form 1 | Form 1 | Form 1 |
| | Solution | Slurry | | | | | | | |
| Tetrahydrofuran | Clear | Thick | Heptane | N/A | N/A | Slurry | Form 3 | Form 4 | Amorphous |
| | Solution | Slurry | | | | | | | |

The osanetant p-toluenesulfonic acid Form 1 solids consisted of aggregated crystals with an undefined morphology. The X-ray powder diffraction (XRPD) pattern for osanetant p-toluenesulfonic acid Form 1 is shown in FIG. 7. Select peaks which can be used to characterize osanetant p-toluenesulfonic acid Form 1 include one or more of 11.4°2θ±0.2°2θ, 18.0°2θ±0.2°2θ, 19.0°2θ±0.2°2θ, 19.3°2θ±0.2°2θ, 19.8°2θ±0.2°2θ, or 21.1°2θ±0.2°2θ. In some embodiments, osanetant p-toluenesulfonic acid Form 1 is a monohydrate. Form 1 (monohydrate) was physically stable in slurry at various water activity and pH, and at 40° C./75% RH and 80° C. Further, the hydrate did not lose the water on dynamic vapor sorption (DVS) analysis.

The osanetant p-toluenesulfonic acid Form 2 solids consisted of aggregated crystals with an undefined morphology. The X-ray powder diffraction (XRPD) pattern for osanetant p-toluenesulfonic acid Form 2 is shown in FIG. 8. Select peaks which can be used to characterize osanetant p-toluenesulfonic acid Form 2 include one or more of 11.2°2θ±0.2°2θ, 11.4°2θ±0.2°2θ, 12.5°2θ±0.2°2θ, 16.5°2θ±0.2°2θ, 19.2°2θ±0.2°2θ, 22.6°2θ±0.2°2θ, or 22.9°2θ±0.2°2θ.

The X-ray powder diffraction (XRPD) pattern for osanetant p-toluenesulfonic acid Form 3 is shown in FIG. 9. Select peaks which can be used to characterize osanetant p-toluenesulfonic acid Form 3 include one or more of 5.9°2θ±0.2°2θ, 6.1°274 ±0.2°2θ, 15.5°2θ±0.2°2θ, 18.4°2θ±0.2°2θ, 19.2°2θ±0.2°2θ, or 21.0°2θ±0.2°2θ.

The osanetant p-toluenesulfonic acid Form 4 solids consisted of aggregated crystals with an undefined morphology. The X-ray powder diffraction (XRPD) pattern for osanetant p-toluenesulfonic acid Form 4 is shown in FIG. 10. Select peaks which can be used to characterize osanetant p-toluenesulfonic acid Form 4 include one or more of 5.9°2θ±0.2°2θ, 13.3°2θ±0.2°2θ, 13.5°2θ±0.2°2θ, 16.3°2θ±0.2°2θ, 18.4°2θ±0.2°2θ, 19.5°2θ±0.2°2θ, or 22.7°2θ±0.2°2θ.

TG analysis of osanetant p-toluenesulfonic acid Form 1 indicated that there was a loss of 2.4 wt. % on heating to 120° C., and a further loss of 10.8 wt. % up to 260° C. DSC analysis indicated that there was a broad endothermic event (peak at 97.4° C.), and an endothermic event onset 156.3° C. (peak at 163.4° C.). The simultaneous TG/DSC of osanetant p-toluenesulfonic acid Form 1 is shown in FIG. 28.

TG analysis of osanetant p-toluenesulfonic acid Form 4 indicated that there was a loss of 4.2 wt. % on heating to 140° C., and a further loss of 10.0 wt. % up to 260° C. DSC analysis indicated that there was a small exothermic event with onset 137.4° C. (peak at 143.2° C.), and an endothermic event onset 188.4° C. (peak at 193.4° C.). The simultaneous TG/DSC of osanetant p-toluenesulfonic acid Form 4 is shown in FIG. 29.

Two anhydrous forms of the osanetant p-toluenesulfonic acid were synthesized. Form 5 was obtained from de-hydration of Form 1, however was unstable and Form 5 re-hydrated to Form 1 under ambient conditions. Form 6, described below, was a stable anhydrous form, but hydrated in water and aqueous solvent mixtures.

The osanetant p-toluenesulfonic acid Form 6 solids consisted of crystals with laths. The X-ray powder diffraction (XRPD) pattern for osanetant p-toluenesulfonic acid Form 6 is shown in FIG. 39. Select peaks which can be used to characterize osanetant p-toluenesulfonic acid Form 6 include one or more of 14.9°2θ±0.2°2θ, 16.6°2θ±0.2°2θ, 18.2°2θ±0.2°2θ, 18.4°2θ±0.2°2θ, 21.2°2θ±0.2°2θ, and 21.8°2θ±0.2°2θ. Osanetant p-toluenesulfonic acid Form 6 is anhydrous. DSC analysis indicated that there was an endothermic event onset at 193.2° C. (peak at 196.3° C.). The simultaneous TG/DSC of osanetant p-toluenesulfonic acid Form 6 is shown in FIG. 40.

Further data on osanetant p-toluenesulfonic acid Form 1, osanetant p-toluenesulfonic acid Form 2, osanetant p-toluenesulfonic acid Form 4 and osanetant p-toluenesulfonic acid Form 6 is shown in Table 12.

Advantageously, Form 1 (hydrate) and Form 6 (anhydrous) were stable on drying. Form 1 re-hydrated at room temperature and pressure after KF analysis, when it was heated in an oven to remove the water.

TABLE 12

| | | | Simultaneous TG/DSC | | |
|---|---|---|---|---|---|
| XRPD | ¹H NMR | | TG - Mass | | |
| Pattern | Solvent | Counterion | Losses | DSC - Thermal Events | PLM |
| Form 1 | 0.05 eq. | Approx. 1 | 2.4 wt. % up | Broad Endothermic event (peak at 97.4° C.), | Small |
| | IPA | eq. | to 120° C. | Endothermic event onset 156.3° C. (Peak at | Aggregated |
| | | | 10.8 wt. % up | 163.4° C.) | |
| | | | to 260° C. | | |
| Form 2 | 0.04 eq. | Approx. 1 | 2.7 wt. % up | Broad Endothermic event (peak at 90.0° C.), | Small |
| | Ethanol | eq. | to 125° C. | Endothermic event onset 151.8° C. (Peak at | Aggregated |
| | | | 11.0 wt. % up | 158.5° C.) | |
| | | | to 260° C. | | |
| Form 4 | 0.25 eq. | Approx. 1 | 4.2 wt. % up | Small Exothermic event Onset 137.4° C. (Peak | Small |
| | THF | eq. | to 140° C. | at 143.2° C.), Endothermic event onset | Aggregated |
| | | | 10.0 wt. % up | 188.4° C. (Peak at 193.4° C.) | |
| | | | to 260° C. | | |
| Form 6 | Anhydrous | Approx. 1 | 0.3 wt. % up | Endothermic peak onset at 193.2° C. (peak at | Laths |
| | | eq. | to 140° C. | 196.3° C.). | |
| | | | 9.6 wt. % up | | |
| | | | to 260° C. | | |

### Naphthalene-2-sulfonic Acid

The following observations were made using naphthalene-2-sulfonic acid as the counterion in Method A. After 3 days of thermal cycling, clear solutions were observed in two of the solvent systems, and slurries in four. After anti-solvent addition and further thermal cycling, slurries were observed. Crystalline material was observed from four of the solvent systems. Patterns 1, 2, 3 and 4 were retained on drying at 40° C. and 40° C./75% RH. Patterns 1-4 were assigned Forms 1-4, respectively. Results are summarized in Table 13.

TABLE 13

| | | | Anti-Solvent Addition | | | Observation | XRPD | | |
|---|---|---|---|---|---|---|---|---|---|
| | Observation | | Anti- | Volume/ | | 4 Day | | | 40° C./ |
| Solvent | Initial | 3 Day | Solvent | mL | Observation | (total) | Wet | 40° C. | 75% RH |
| 2-Propanol:Water | Clear | Clear | Water | 0.3 | Slurry | Gum | Amorphous | Amorphous | Amorphous |
| 75:25 % v/v | Solution | Solution | | | | | | | |
| Acetone | Clear | Clear | Heptane | 1 | Clear | Gum | Amorphous | Amorphous | Amorphous |
| | Solution | Solution | | | Solution | | | | |
| Acetonitrile | Clear | Slurry | N/A | N/A | N/A | Slurry | Form 1 | Form 1 | Form 1 |
| | Solution | | | | | | | | |
| Ethanol | Clear | Slurry | N/A | N/A | N/A | Slurry | Form 2* | Form 2* | Form 2* |
| | Solution | | | | | | | | |
| Ethyl Acetate | Clear | Thick | N/A | N/A | N/A | Slurry | Form 3 | Form 3 | Form 3 |
| | Solution | Slurry | | | | | | | |
| Tetrahydrofuran | Clear | Slurry | N/A | N/A | N/A | Slurry | Form 4 | Form 4 | Form 4 |
| | Solution | | | | | | | | |

*= Poorly Crystalline

The osanetant naphthalene-2-sulfonic acid Form 1 solids consisted of small lath like crystals. The X-ray powder diffraction (XRPD) pattern for osanetant naphthalene-2-sulfonic acid Form 1 is shown in FIG. 11. Select peaks which can be used to characterize osanetant naphthalene-2-sulfonic acid Form 1 include one or more of 5.7°2θ±0.2°2θ, 14.8°2θ±0.2°2θ, 15.6°2θ±0.2°2θ, 16.5°2θ±0.2°2θ, 17.0°2θ±0.2°2θ, 19.3°θ0±0.2°2θ, or 22.0°2θ±0.2°2θ.

The osanetant naphthalene-2-sulfonic acid Form 2 solids consisted of small lath like crystals with aggregation. The X-ray powder diffraction (XRPD) pattern for osanetant naphthalene-2-sulfonic acid Form 2 is shown in FIG. 12. Select peaks which can be used to characterize osanetant naphthalene-2-sulfonic acid Form 2 include one or more of 5.6°2θ±0.2°2θ, 15.1°2θ±0.2°2θ, 16.1°2θ±0.2°2θ, 16.8°2θ±0.2°2θ, 18.9°2θ±0.2°2θ, or 19.2°2θ±0.2°2θ.

The osanetant naphthalene-2-sulfonic acid Form 3 solids consisted of small undefined aggregates. The X-ray powder diffraction (XRPD) pattern for osanetant naphthalene-2-sulfonic acid Form 3 is shown in FIG. 13. Select peaks which can be used to characterize osanetant naphthalene-2-sulfonic acid Form 3 include one or more of 6.4°2θ±0.2°2θ, 14.6°2θ±0.2°2θ, 16.3°2θ±0.2°2θ, 16.7°2θ±0.2°2θ, 18.2°2θ±0.2°2θ, or 19.2°2θ±0.2°2θ.

The osanetant naphthalene-2-sulfonic acid Form 4 solids consisted of small aggregated crystals. The X-ray powder diffraction (XRPD) pattern for osanetant naphthalene-2-sulfonic acid Form 4 is shown in FIG. 14. Select peaks which can be used to characterize osanetant naphthalene-2-sulfonic acid Form 4 include one or more of 5.8°2θ±0.2°2θ, 6.0°2θ±0.2°2θ, 15.4°2θ±0.2°2θ, 18.2°2θ±0.2°2θ, 19.0°2θ±0.2°2θ, 19.9°2θ±0.2°2θ, or 21.2°2θ±0.2°2θ.

TG analysis of osanetant naphthalene-2-sulfonic acid Form 1 indicated that there was a loss of 3.6 wt. % on heating to 155° C., and a further loss of 9.5 wt. % on heating to 260° C. DSC analysis indicated that there was overlapping endothermic events with peaks at 137.4° C. and 153.6° C. The simultaneous TG/DSC of osanetant naphthalene-2-sulfonic acid Form 1 is shown in FIG. 30.

TG analysis of osanetant naphthalene-2-sulfonic acid Form 2 indicated that there was a loss of 3.6 wt. % on heating to 140° C., and a further loss of 9.8 wt. % on heating to 260° C. DSC analysis indicated that there was an endothermic event with an onset of 143.9° C. (peak at 156.8° C.). The simultaneous TG/DSC of osanetant naphthalene-2-sulfonic acid Form 2 is shown in FIG. 31.

TG analysis of osanetant naphthalene-2-sulfonic acid Form 3 indicated that there was a loss of 0.9 wt. % on heating to 150° C., and a further loss of 9.6 wt. % on heating to 260° C. DSC analysis indicated that there was an endothermic event with an onset of 170.5° C. (peak at 175.5° C.). The simultaneous TG/DSC of osanetant naphthalene-2-sulfonic acid Form 3 is shown in FIG. 32.

TG analysis of osanetant naphthalene-2-sulfonic acid Form 4 indicated that there was a loss of 5.6 wt. % on heating to 165° C., and a further loss of 9.1 wt. % on heating to 250° C. DSC analysis showed a poor thermal profile. The simultaneous TG/DSC of osanetant naphthalene-2-sulfonic acid Form 4 is shown in FIG. 33.

Further data on osanetant naphthalene-2-sulfonic acid Form 1, osanetant naphthalene-2-sulfonic acid Form 2, osanetant naphthalene-2-sulfonic acid Form 3, and osanetant naphthalene-2-sulfonic acid Form 4 is shown in Table 14.

TABLE 14

| | ¹H NMR | | Simultaneous TG/DSC | | |
|---|---|---|---|---|---|
| XRPD Pattern | Solvent | Counterion | TG - Mass Losses | DSC - Thermal Events | PLM |
| Form 1 | 0.3 eq. ACN | 0.9 eq. | 3.6 wt. % up to | Overlapping endothermic | Small Laths |
| | | | 155° C., | events onset | |
| | | | 9.5 wt. % up to | 132.0° C. (peaks at 137.4 and | |
| | | | 260° C. | 153.6° C.) | |
| Form 2 | Trace Solvent | 0.9 eq. | 3.4 wt. % up to | Endothermic event onset | Small |
| | | | 140° C., | 143.9° C. (peak at 156.8° C.) | Laths, |
| | | | 9.8 wt. % up to | | Aggregated |
| | | | 260° C. | | |
| Form 3 | Trace Solvent | 0.9 eq. | 0.9 wt. % up to | Endothermic event onset | Small |
| | | | 150° C., | 170.5° C. (peak at 175.5° C.) | Aggregated |
| | | | 9.6 wt. % up to | | |
| | | | 260° C. | | |
| Form 4 | 0.7 eq. THF | 0.9 eq. | 5.6 wt. % up to | Poorly defined endothermic | Small |
| | | | 165° C., | events with peaks at 126.3 | Aggregated |
| | | | 9.1 wt. % up to | and 136.0° C. | |
| | | | 250° C. | | |

### Benzenesulfonic Acid

The following observations were made using benzenesulfonic acid as the counterion in Method A. After 2 days of thermal cycling, clear solutions were observed in two of the solvent systems, and slurries in four. After anti-solvent addition and further thermal cycling, slurries were observed. Crystalline material was observed form all of the solvent systems. Four different XRPD patterns were observed. After drying at 40° C., there was no change in form, however there was a reduction in crystallinity of Pattern 2. After drying at 40° C./75% RH. Pattern 1 and Pattern 3 were retained. Patterns 2 and 4 converted to Pattern 1. Patterns 1-4 were assigned Forms 1-4, respectively. Results are summarized in Table 15.

TABLE 15

| | | | Anti-Solvent Addition | | | Observation | XRPD | | |
|---|---|---|---|---|---|---|---|---|---|
| | Observation | | Anti- | Volume/ | | 3 Day | | | 40° C./ |
| Solvent | Initial | 2 Day | Solvent | mL | Observation | (total) | Wet | 40° C. | 75% RH |
| 2-Propanol:Water | Clear | Slurry | N/A | N/A | N/A | Slurry | Form 1 | Form 1 | Form 1 |
| 75:25 % v/v | Solution | | | | | | | | |
| Acetone | Clear | Slurry | N/A | N/A | N/A | Slurry | Form 1 | Form 1 | Form 1 |
| | Solution | | | | | | | | |
| Acetonitrile | Clear | Clear | Water | 1 | Slurry | Slurry | Form 1 | Form 1 | Form 1 |
| | Solution | Solution | | | | | | | |
| Ethanol | Clear | Clear | Heptane | 1 | Slurry | Slurry | Form 2 | 2* | Form 1 |
| | Solution | Solution | | | | | | | |
| Ethyl Acetate | Hazy | Slurry | N/A | N/A | N/A | Slurry | Form 3 | Form 3 | Form 3 |
| Tetrahydrofuran | Clear | Slurry | N/A | N/A | N/A | Slurry | Form 4 | Form 4 | Form 1** |
| | Solution | | | | | | | | |

*= Poorly Crystalline;
**= Some Minor Differences

The osanetant benzenesulfonic acid Form 1 solids consisted of fractured crystals with a variation in size. There were some large crystals. The X-ray powder diffraction (XRPD) pattern for osanetant benzenesulfonic acid Form 1 is shown in FIG. 15. Select peaks which can be used to characterize osanetant benzenesulfonic acid Form 1 include one or more of 11.3°2θ±0.2°2θ, 14.9°2θ+0.2°2θ, 16.7°2θ±0.2°2θ, 17.9°2θ±0.2°2θ, 18.6°2θ±0.2°2θ, or 19.3°2θ±0.2°2θ. The simultaneous TG/DSC of osanetant benzene sulfonic acid Form 1 is shown in FIG. 41. In some embodiments, the osanetant benzenesulfonic acid Form 1 is a monohydrate.

The osanetant benzenesulfonic acid Form 2 solids consisted of aggregated small crystals. The X-ray powder diffraction (XRPD) pattern for osanetant benzenesulfonic acid Form 2 is shown in FIG. 16. Select peaks which can be used to characterize osanetant benzenesulfonic acid Form 2 include one or more of 7.8°2θ±0.2°2θ, 12.2°2θ±0.2°2θ, 15.6°2θ±0.2°2θ, 16.3°2θ±0.2°2θ, 16.6°2θ±0.2°2θ, 20.0°2θ±0.2°2θ, or 22.5°2θ±0.2°2θ.

The osanetant benzenesulfonic acid Form 3 solids consisted of small aggregated crystals. The X-ray powder diffraction (XRPD) pattern for osanetant benzenesulfonic acid Form 3 is shown in FIG. 17. Select peaks which can be used to characterize osanetant benzenesulfonic acid Form 3 include one or more of 12.8°2θ±0.2°2θ, 16.4°2θ±0.2°2θ, 17.4°2θ±0.2°2θ, 18.3°2θ±0.2°2θ, 21.4°2θ±0.2°2θ, or 21.8°2θ±0.2°2θ. In some embodiments, osanetant benzenesulfonic acid Form 3 is anhydrous.

The osanetant benzenesulfonic acid Form 4 solids consisted of small aggregated. The X-ray powder diffraction (XRPD) pattern for osanetant benzenesulfonic acid Form 4 is shown in FIG. 18. Select peaks which can be used to characterize osanetant benzenesulfonic acid Form 4 include one or more of 5.0°2θ±0.2°2θ, 15.1°2θ±0.2°2θ, 16.2°2θ±0.2°2θ, 18.2°2θ±0.2°2θ, 20.0°2θ±0.2°2θ, 20.5°2θ±0.2°2θ, or 21.1°2θ±0.2°2θ.

TG analysis osanetant benzenesulfonic acid Form 3 indicated that there was a loss of 0.6 wt. % on heating to 155° C., and a further loss of 11.3 wt. % on heating to 265° C. DSC analysis indicated that there was an endothermic event with an onset of 176.8° C. (peak at 181.0° C.). The simultaneous TG/DSC of osanetant benzenesulfonic acid Form 3 is shown in FIG. 34.

Further data on osanetant benzenesulfonic acid Form 1, osanetant benzenesulfonic acid Form 2, osanetant benzenesulfonic acid Form 3, and osanetant benzenesulfonic acid Form 4 is shown in Table 16. Hydrate and anhydrous benzene sulfonic acid forms were stable on drying. Benzenesulfonic acid forms showed higher solubilities at pH 1.2 and 4.5 than the p-toluenesulfonic acid forms.

TABLE 16

| XRPD | ¹H NMR | | Simultaneous TG/DSC | | |
|---|---|---|---|---|---|
| Pattern | Solvent | Counterion | TG - Mass Losses | DSC - Thermal Events | PLM |
| Form 1 | 0.05 eq. | Ca. 1 eq. | 2.6 wt. % up to | Endothermic event onset | Variation in size |
| | IPA | | 145° C., | 149.9° C. (peak | |
| | | | 11.9 wt. % up to | at 159.7° C.) | |
| | | | 260° C. | | |
| Form 1 | Trace | Ca. 1.2 eq | 2.4 wt. % up to | Endothermic event onset at | Aggregated small |
| | Solvent | | 145° C., | 147.6° C. (peak | laths |
| | | | 7.9 wt. % up to | at 156.3° C.) | |
| | | | 260° C. | | |
| Form 2 | Trace | Ca. 1 eq | 1.3 wt. % up to | Small Exothermic event Onset | Small Aggregated |
| | Solvent | | 150° C., | 118.5° C. (peak at 124.9° C.), | |
| | | | 10.9 wt. % up to | Endothermic event onset 139.1° C. | |
| | | | 265° C. | (peak at 149.0° C.) | |
| Form 3 | Trace | Ca. 1 eq. | 0.6 wt. % up to | Endothermic event onset | Small Aggregated |
| | Solvent | | 155° C., | 176.8° C. (peak | |
| | | | 11.3 wt. % up to | at 181.0° C.) | |
| | | | 265° C. | | |
| Form 4 | 0.4 eq. | Ca. 1 eq | 4.8 wt. % up to | Poorly defined Endothermic | Small Aggregated |
| | THF | | 150° C., | events with peaks at | |
| | | | 12.6 wt. % up to | 114.1 and 147.7° C. | |
| | | | 260° C. | | |

### Maleic Acid

The following observations were made using maleic acid as the counterion in Method A. After 2 days of thermal cycling, clear solutions were observed in all of the solvent systems. After anti-solvent addition and further thermal cycling, gums and thin slurries were observed. Results are summarized in Table 17.

TABLE 17

| | | | Anti-Solvent Addition | | | Observation |
|---|---|---|---|---|---|---|
| | Observation | | Anti- | Volume/ | | 3 Day |
| Solvent | Initial | 2 Day | Solvent | mL | Observation | (total) |
| 2-Propanol:Water | Clear | Clear | Water | 0.6 | Slurry | Thin |
| 75:25 % v/v | Solution | Solution | | | | Slurry |
| Acetone | Clear | Clear | Heptane | 0.8 | Slurry | Gum |
| | Solution | Solution | | | | |
| Acetonitrile | Clear | Clear | Water | 1 | Slurry | Thin |
| | Solution | Solution | | | | Slurry |
| Ethanol | Clear | Clear | Heptane | 0.8 | Slurry | Gum |
| | Solution | Solution | | | | |
| Ethyl Acetate | Clear | Clear | Heptane | 0.4 | Slurry | Gum |
| | Solution | Solution | | | | |
| Tetrahydrofuran | Clear | Clear | Heptane | 0.4 | Slurry | Gum |
| | Solution | Solution | | | | |

### Phosphoric Acid

The following observations were made using phosphoric acid as the counterion in Method A. After 2 days of thermal cycling, clear solutions were observed in two of the solvent systems. Three different XRPD patterns were observed form the wet solids. Pattern 1 and 2 were very similar, however there were some differences in peak position. All of the patterns were retained on drying at 40° C. After drying at 40° C./75% RH, Pattern 3 was retained, and Patterns 1 and 2 converted to Pattern 4. Pattern 4 was very similar to Patterns 1 and 2. Patterns 1-4 were assigned Forms 1-4, respectively. Results are summarized in Table 18. Pattern 4 was found to be difficult to reproduce, and an additional Pattern 7 was obtained. Phosphate Pattern 7 was a hydrated form. Extensive polymorphism was observed. Good solid chemical stability observed. Phosphate Pattern 7 had very high solubility in water and buffered systems at pH 1.2 and 4.5. Disproportionation was observed in pH 6.8 buffer. Pattern 7 was assigned Form 7.

Select XRPD peaks which can be used to characterize osanetant phosphate Form 7 include one or more of 11.4°2θ±0.2°2θ, 15.1°2θ±0.2°2θ, 19.1°2θ±0.2°2θ, 19.5°2θ±0.2°2θ, 22.8°2θ±0.2°2θ, and 23.1°2θ±0.2°2θ.

TABLE 18

| | | | Anti-Solvent Addition | | | Observation | XRPD | | |
|---|---|---|---|---|---|---|---|---|---|
| | Observation | | Anti- | Volume/ | | 3 Day | | | 40° C./ |
| Solvent | Initial | 2 Day | Solvent | mL | Observation | (total) | Wet | 40° C. | 75% RH |
| 2-Propanol:Water | Clear | Clear | Water | 1 | Clear | Clear | N/A | N/A | N/A |
| 75:25 % v/v | Solution | Solution | | | Solution | Solution | | | |
| Acetone | Gum | Slurry | N/A | N/A | N/A | Slurry | Form 1 | Form 1 | Form 4 |
| Acetonitrile | Gum | Gum | N/A | N/A | N/A | Thin | N/A | N/A | N/A |
| | | | | | | Slurry | | | |
| Ethanol | Gum | Slurry | N/A | N/A | N/A | Slurry | Form 2 | Form 2 | Form 4* |
| Ethyl Acetate | Gum | Clear | Heptane | 0.8 | Slurry | Gum | N/A | N/A | N/A |
| | | Solution | | | | | | | |
| Tetrahydrofuran | Clear | Slurry | N/A | N/A | N/A | Slurry | Form 3 | Form 3 | Form 3 |
| | Solution | | | | | | | | |

*= Poorly Crystalline

The osanetant phosphoric acid Form 1 solids consisted of aggregated crystals with a variation in size. The X-ray powder diffraction (XRPD) pattern for osanetant phosphoric acid Form 1 is shown in FIG. 19. Select peaks which can be used to characterize osanetant phosphoric acid Form 1 include one or more of 6.0°2θ±0.2°2θ, 6.1°2θ±0.2°2θ, 11.3°2θ±0.2°2θ, 17.9°2θ±0.2°2θ, 20.0°2θ±0.2°2θ, or 21.0°2θ±0.2°2θ.

The osanetant phosphoric acid Form 2 solids consisted of small aggregated crystals. The X-ray powder diffraction (XRPD) pattern for osanetant phosphoric acid Form 2 is shown in FIG. 20. Select peaks which can be used to characterize osanetant phosphoric acid Form 2 include one or more of 5.9°2θ±0.2°2θ, 6.2°2θ±0.2°2θ, 11.4°2θ±0.2°2θ, 17.9°2θ±0.2°2θ, 19.0°2θ±0.2°2θ, or 19.5°2θ±0.2°2θ.

The osanetant phosphoric acid Form 3 solids consisted of small aggregated. The X-ray powder diffraction (XRPD) pattern for osanetant phosphoric acid Form 3 is shown in FIG. 21. Select peaks which can be used to characterize osanetant phosphoric acid Form 3 include one or more of 6.1°2θ±0.2°2θ, 11.2°2θ±0.2°2θ, 15.5°2θ±0.2°2θ, 17.5°2θ±0.2°2θ, 18.4°2θ±0.2°2θ, 19.8°2θ±0.2°2θ, or 20.7°2θ±0.2°2θ.

The X-ray powder diffraction (XRPD) pattern for osanetant phosphoric acid Form 4 is shown in FIG. 22. Select peaks which can be used to characterize osanetant phosphoric acid Form 4 include one or more of 5.9°2θ±0.2°2θ, 11.5°2θ±0.2°2θ, 16.1°2θ±0.2°2θ, 17.9°2θ±0.2°2θ, 19.0°2θ±0.2°2θ, 19.5°2θ±0.2°2θ, or 21.1°2θ±0.2°2θ.

TG analysis of osanetant phosphoric acid Form 3 showed a small loss of 0.4 wt. % on heating to 100° C., and a loss of 7.8 wt. % on heating to 175° C., followed by a loss of 12.8 wt. % on heating to 250° C. DSC analysis indicated that there was a small endothermic event with onset 139.4° C. (peak at 143.9° C.). The simultaneous TG/DSC of osanetant phosphoric acid Form 3 is shown in FIG. 35.

Further data on osanetant phosphoric acid Form 1, osanetant phosphoric acid Form 2, and osanetant phosphoric acid Form 3 is shown in Table 19.

TABLE 19

| XRPD | ¹H NMR | | Simultaneous TG/DSC | | |
|---|---|---|---|---|---|
| Pattern | Solvent | Counterion | TG - Mass Losses | DSC - Thermal Events | PLM |
| Form 1 | 0.5 eq. | Shifts in comparison | 6.6 wt. % up to 175° C. | Poorly defined - | Variation |
| | Acetone | to FB | 12.0 wt. % up | Endothermic event with | in size |
| | | | to 250° C. | peaks at 147.6° C. | |
| Form 2 | 0.2 eq. | Shifts in comparison | 5.5 wt. % up to 70° C. | Broad Endothermic event | Small |
| | Ethanol | to FB | 0.8 wt. % up to 160° C. | onset 150.8° C. | Aggregated |
| | | | 14.2 wt. % up | (peak at 165.5° C.) | |
| | | | to 250° C. | | |
| Form 3 | 0.5 eq. THF | Shifts in comparison | 0.4 wt. % up to 90° C. | Poorly defined - | Small |
| | | to FB | 7.8 wt. % up to 175° C. | Endothermic event onset | Aggregated |
| | | | 12.8 wt. % up | 139.4 (peak at 143.9° C.). | |
| | | | to 250° C. | | |

### Glutamic Acid

The following observations were made using glutamic acid as the counterion in Method A. After 2 days of thermal cycling, slurries were observed in five of the solvent systems. After anti-solvent addition and further thermal cycling of the 2-propanol: water experiment, a thin slurry was observed. The solids obtained from the slurries were consistent with glutamic acid on XRPD analysis. Results are summarized in Table 20.

TABLE 20

| | | | Anti-Solvent Addition | | | Observation | |
|---|---|---|---|---|---|---|---|
| | Observation | | Anti- | Volume/ | | 3 Day | XRPD |
| Solvent | Initial | 2 Day | Solvent | mL | Observation | (total) | Wet |
| 2-Propanol:Water | Clear | Clear | Water | 0.8 | Slurry | Thin | N/A |
| 75:25 % v/v | Solution | Solution | | | | Slurry | |
| Acetone | Hazy | Slurry | N/A | N/A | N/A | Slurry | |
| Acetonitrile | Hazy | Slurry | N/A | N/A | N/A | Slurry | |
| Ethanol | Hazy | Slurry | N/A | N/A | N/A | Slurry | |
| Ethyl Acetate | Hazy | Slurry | N/A | N/A | N/A | Slurry | |
| Tetrahydrofuran | Hazy | Slurry | N/A | N/A | N/A | Slurry | |

### 1-Hydroxy-2-naphthoic Acid

The following observations were made using 1-hydroxy-2-naphthoic acid as the counterion in Method A. After 2 days of thermal cycling, clear and hazy solutions were observed. After anti-solvent addition, slurries were observed in five of the experiments, however the material oiled out. A thin faint brown slurry was observed form IPA: water. This was likely free counterion, as the 1-hydroxy-2-naphthoic acid was faint brown. Results are summarized in Table 21.

TABLE 21

| | | | Anti-Solvent Addition | | | Observation | |
|---|---|---|---|---|---|---|---|
| | Observation | | Anti- | Volume/ | | 3 Day | XRPD |
| Solvent | Initial | 2 Day | Solvent | mL | Observation | (total) | Wet |
| 2-Propanol:Water | Hazy | Hazy | Water | 0.4 | Slurry | Thin Faint | N/A |
| 75:25 % v/v | | | | | | Brown Slurry | |
| Acetone | Clear | Clear | Heptane | 1 | Hazy | Brown Oil | N/A |
| | Solution | Solution | | | | | |
| Acetonitrile | Hazy | Clear | Water | 0.8 | Slurry | Brown Oil | N/A |
| | | Solution | | | | | |
| Ethanol | Hazy | Hazy | Heptane | 1 | Slurry | Brown Oil | N/A |
| Ethyl Acetate | Clear | Clear | Heptane | 0.6 | Slurry | Brown Oil | N/A |
| | Solution | Solution | | | | | |
| Tetrahydrofuran | Clear | Clear | Heptane | 0.6 | Slurry | Brown Oil | N/A |
| | Solution | Solution | | | | | |

### Malonic Acid

The following observations were made using malonic acid as the counterion in Method A. After 2 days of thermal cycling, clear solutions were observed. Slurries were observed from five of the systems after anti-solvent addition. Solids were not obtained. Results are summarized in Table 22.

TABLE 22

| | | | Anti-Solvent Addition | | | Observation | |
|---|---|---|---|---|---|---|---|
| | Observation | | Anti- | Volume/ | | 3 Day | XRPD |
| Solvent | Initial | 2 Day | Solvent | mL | Observation | (total) | Wet |
| 2-Propanol:Water | Clear | Clear | Water | 1 | Hazy | Thin | N/A |
| 75:25 % v/v | Solution | Solution | | | | Slurry | |
| Acetone | Clear | Clear | Heptane | 0.8 | Slurry | Gum | N/A |
| | Solution | Solution | | | | | |
| Acetonitrile | Clear | Clear | Water | 1 | Clear | Clear | N/A |
| | Solution | Solution | | | Solution | Solution | |
| Ethanol | Clear | Clear | Heptane | 1 | Slurry | Gum | N/A |
| | Solution | Solution | | | | | |
| Ethyl Acetate | Clear | Clear | Heptane | 0.4 | Slurry | Gum | N/A |
| | Solution | Solution | | | | | |
| Tetrahydrofuran | Clear | Clear | Heptane | 0.4 | Slurry | Gum | N/A |
| | Solution | Solution | | | | | |

### Gentisic Acid

The following observations were made using gentisic acid as the counterion in Method A. After 2 days of thermal cycling, clear solutions were observed. Slurries were observed after anti-solvent addition. XRPD analysis indicated that the recovered solids were amorphous, with no change on drying at 40° C. and 40° C./75% RH. After evaporation form ethanol: heptane, poorly crystalline material was obtained. Results are summarized in Table 23.

TABLE 23

| | | | Anti-Solvent Addition | | | Observation | XRPD | | |
|---|---|---|---|---|---|---|---|---|---|
| | Observation | | Anti- | Volume/ | | 3 Day | | | 40° C./ |
| Solvent | Initial | 2 Day | Solvent | mL | Observation | (total) | Wet | 40° C. | 75% RH |
| 2-Propanol:Water | Clear | Clear | Water | 0.6 | Slurry | Gum | Amorphous | Amorphous | Amorphous |
| 75:25 % v/v | Solution | Solution | | | | | | | |
| Acetone | Clear | Clear | Heptane | 0.6 | Slurry | Gum | N/A | N/A | N/A |
| | Solution | Solution | | | | | | | |
| Acetonitrile | Clear | Clear | Water | 0.8 | Slurry | Thin | N/A | N/A | N/A |
| | Solution | Solution | | | | Slurry | | | |
| Ethanol | Clear | Clear | Heptane | 0.6 | Slurry | Gum | Poorly | N/A | N/A |
| | Solution | Solution | | | | | Crystalline | | |
| Ethyl Acetate | Hazy | Clear | Heptane | 0.2 | Slurry | Solids | Amorphous | Amorphous | Amorphous |
| | | Solution | | | | | | | |
| Tetrahydrofuran | Clear | Clear | Heptane | 0.4 | Slurry | Solids | Amorphous | Amorphous | Amorphous |
| | Solution | Solution | | | | | | | |

### L-Tartaric Acid

The following observations were made using L-tartaric acid as the counterion in Method A. After 2 days of thermal cycling, two slurries were observed. Anti-solvent addition produced slurries in three of the four experiments. Poorly crystalline material was observed from acetonitrile and ethanol, with two different XRPD patterns, osanetant L-tartaric acid Form 1 and osanetant L-tartaric acid Form 2, respectively. There was no change on drying at 40° C., however any crystallinity was lost at 40° C./75% RH. Amorphous material was recovered from three of the experiments. Results are summarized in Table 24.

TABLE 24

| | | | Anti-Solvent Addition | | | Observation | XRPD | | |
|---|---|---|---|---|---|---|---|---|---|
| | Observation | | Anti- | Volume/ | | 3 Day | | | 40° C./ |
| Solvent | Initial | 2 Day | Solvent | mL | Observation | (total) | Wet | 40° C. | 75% RH |
| 2-Propanol:Water | Clear | Clear | Water | 1 | Slurry | Gum | N/A | N/A | N/A |
| 75:25 % v/v | Solution | Solution | | | | | | | |
| Acetone | Clear | Clear | Heptane | 1 | Gum | Slurry | Amorphous | Amorphous | Amorphous |
| | Solution | Solution | | | | | | | |
| Acetonitrile | Clear | Slurry | N/A | N/A | N/A | Slurry | Form 1* | Form 1* | Amorphous |
| | Solution | | | | | | | | |
| Ethanol | Clear | Slurry | N/A | N/A | N/A | Slurry | Form 2* | Form 2* | Amorphous |
| | Solution | | | | | | | | |
| Ethyl Acetate | Counterion | Clear | Heptane | 0.4 | Slurry | Sticky | Amorphous | Amorphous | Amorphous |
| | | Solution | | | | Solids | | | |
| Tetrahydrofuran | Clear | Clear | Heptane | 0.4 | Slurry | Thick | Amorphous | Amorphous | Amorphous |
| | Solution | Solution | | | | Slurry | | | |

*= Poorly Crystalline

The osanetant L-tartaric acid Form 1 solids consisted of large particles. The osanetant L-tartaric acid Form 2 solids consisted of acicular rods. Further data on osanetant L-tartaric acid Form 1 and osanetant L-tartaric acid Form 2 is shown in Table 25.

TABLE 25

| | | | Simultaneous TG/DSC | | |
|---|---|---|---|---|---|
| XRPD | ¹H NMR | | TG - Mass | | |
| Pattern | Solvent | Counterion | Losses | DSC - Thermal Events | PLM |
| Form 1 | 1 eq. ACN | 0.8 eq. | 5.1 wt. % up to | Broad Endothermic event onset | Large Crystals |
| | | | 140° C., | 99.1° C. (peak at 103.0° C.), | |
| | | | 29.9 wt. % up to | Broad Endothermic event onset | |
| | | | 265° C. | 132.1° C. (peak at 143.4° C.) | |
| Form 2 | 0.8 eq. | 1 eq. | 3.1 wt. % up to | Broad Endothermic event onset | Acicular Rods |
| | Ethanol | | 150° C., | 105.0° C. (peak at 135.8° C.), | |
| | | | 29.9° C. up to | Broad Endothermic event onset | |
| | | | 275° C. | 137.0° C. (peak at 151.1° C.) | |

### Fumaric Acid

The following observations were made using fumaric acid as the counterion in Method A. Poorly crystalline material was obtained from three of the experiments. The solids all had the same XRPD pattern, Pattern 1 (referred to as Form 1). There was no change on drying at 40° C., however the crystallinity was lost on drying at 40° C./75% RH. Results are summarized in Table 26.

TABLE 26

| | | | Anti-Solvent Addition | | | Observation | XRPD | | |
|---|---|---|---|---|---|---|---|---|---|
| | Observation | | Anti- | Volume/ | | 5 Day | | | 40° C./ |
| Solvent | Initial | 4 Day | Solvent | mL | Observation | (total) | Wet | 40° C. | 75% RH |
| 2-Propanol:Water | Clear | Clear | Water | 0.6 | Slurry | Gum | N/A | N/A | N/A |
| 75:25 % v/v | Solution | Solution | | | | | | | |
| Acetone | Clear | Thin | Heptane | 0.4 | Slurry | Slurry | Form 1 | Form 1 | Amorphous |
| | Solution | Slurry | | | | | | | |
| Acetonitrile | Clear | Thin | Water | 1.0 | Slurry | Thin | N/A | N/A | N/A |
| | Solution | Slurry | | | | Slurry | | | |
| Ethanol | Clear | Clear | Heptane | 0.6 | Slurry | Gum | N/A | N/A | N/A |
| | Solution | Solution | | | | | | | |
| Ethyl Acetate | Counterion | Slurry | Heptane | 0.2 | Slurry | Slurry | Form 1 | Form 1 | Amorphous |
| Tetrahydrofuran | Clear | Clear | Heptane | 0.4 | Slurry | Slurry | Form 1 | Form 1 | Amorphous |
| | Solution | Solution | | | | | | | |

* = Poorly Crystalline

### Citric Acid

The following observations were made using citric acid as the counterion in Method A. Gums were obtained from five of the solvent systems after anti-solvent addition and thermal cycling. Amorphous material was obtained from THF: heptane. The material did not crystallize on drying at 40° C. and 40° C./75% RH. Results are summarized in Table 27.

TABLE 27

| | | | Anti-Solvent Addition | | | Observation | XRPD | | |
|---|---|---|---|---|---|---|---|---|---|
| | Observation | | Anti- | Volume/ | | 5 Day | | | 40° C./ |
| Solvent | Initial | 4 Day | Solvent | mL | Observation | (total) | Wet | 40° C. | 75% RH |
| 2-Propanol:Water | Clear | Clear | Water | 0.6 | Slurry | Gum | N/A | N/A | N/A |
| 75:25 % v/v | Solution | Solution | | | | | | | |
| Acetone | Clear | Clear | Heptane | 0.4 | Slurry | Gum | N/A | N/A | N/A |
| | Solution | Solution | | | | | | | |
| Acetonitrile | Clear | Gum | Water | 1.0 | Slurry | Gum | N/A | N/A | N/A |
| | Solution | | | | | | | | |
| Ethanol | Clear | Clear | Heptane | 0.4 | Slurry | Gum | N/A | N/A | N/A |
| | Solution | Solution | | | | | | | |
| Ethyl Acetate | Clear | Clear | Heptane | 0.4 | Slurry | Gum | N/A | N/A | N/A |
| | Solution | Crystals | | | | | | | |
| Tetrahydrofuran | Clear | Clear | Heptane | 0.4 | Thin | Solids | Amorphous | Amorphous | Amorphous |
| | Solution | Solution | | | Slurry | | | | |

### L-Malic Acid

The following observations were made using L-malic acid as the counterion in Method A. Crystalline solids were obtained from thermal cycling in ethyl acetate. There was a change in form on drying at 40° C. The material lost crystallinity on drying at 40° C./75% RH. Amorphous material was isolated from THE: heptane. There was no change on drying at 40° C. and 40° C./75% RH. Results are summarized in Table 28.

TABLE 28

| | | | Anti-Solvent Addition | | | Observation | XRPD | | |
|---|---|---|---|---|---|---|---|---|---|
| | Observation | | Anti- | Volume/ | | 5 Day | | | 40° C./ |
| Solvent | Initial | 4 Day | Solvent | mL | Observation | (total) | Wet | 40° C. | 75% RH |
| 2-Propanol:Water | Clear | Clear | Water | 1.0 | Slurry | Gum | N/A | N/A | N/A |
| 75:25 % v/v | Solution | Solution | | | | | | | |
| Acetone | Clear | Clear | Heptane | 0.4 | Slurry | Gum | N/A | N/A | N/A |
| | Solution | Solution | | | | | | | |
| Acetonitrile | Clear | Clear | Water | 1.0 | Clear | Clear | N/A | N/A | N/A |
| | Solution | Solution | | | Solution | Solution | | | |
| Ethanol | Clear | Clear | Heptane | 0.6 | Slurry | Gum | N/A | N/A | N/A |
| | Solution | Solution | | | | | | | |
| Ethyl Acetate | Clear | Thick | N/A | N/A | N/A | Slurry | Form 1 | Form 2 | Amorphous |
| | Solution | Slurry | | | | | | | |
| Tetrahydrofuran | Clear | Clear | Heptane | 0.8 | Gum | Slurry | Amorphous | Amorphous | Amorphous |
| | Solution | Solution | | | | | | | |

The X-ray powder diffraction (XRPD) pattern for osanetant L-malic acid Form 1 is shown in FIG. 23. Select peaks which can be used to characterize osanetant L-malic acid Form 1 include one or more of 6.2°2θ±0.2°2θ, 11.2°2θ±0.2°2θ, 17.6°2θ±0.2°2θ, 18.8°2θ±0.2°2θ, 19.0°2θ±0.2°2θ, or 22.8°2θ±0.2°2θ.

The osanetant L-malic acid Form 2 solids consisted of irregular crystals with a variation in size. The X-ray powder diffraction (XRPD) pattern for osanetant L-malic acid Form 2 is shown in FIG. 24. Select peaks which can be used to characterize osanetant L-malic acid Form 2 include one or more of 8.0°2θ±0.2°2θ, 11.2°2θ±0.2°2θ, 12.1°2θ±0.2°2θ, 14.8°2θ±0.2°2θ, 18.1°2θ±0.2°2θ, or 19.3°2θ±0.2°2θ.

Further data on osanetant L-tartaric acid Form 1 and osanetant L-tartaric acid Form 2 is shown in Table 29.

TABLE 29

| | | | Simultaneous TG/DSC | | |
|---|---|---|---|---|---|
| XRPD | ¹H NMR | | TG - Mass | DSC - Thermal | |
| Pattern | Solvent | Counterion | Losses | Events | PLM |

| | | | | |
|---|---|---|---|---|
| Form 2 | 0.9 eq. EtOAc | 1.2 eq. | Insufficient Solids for Analysis | Irregular - |

variation in
size

### Hippuric Acid

The following observations were made using hippuric acid as the counterion in Method A. No solids or slurries were obtained from the hippuric acid experiments. Results are summarized in Table 30.

TABLE 30

| | | | Anti-Solvent Addition | | | Observation | |
|---|---|---|---|---|---|---|---|
| | Observation | | Anti- | Volume/ | | 5 Day | XRPD |
| Solvent | Initial | 4 Day | Solvent | mL | Observation | (total) | Wet |
| 2-Propanol:Water | Clear | Clear | Water | 1.0 | Slurry | Gum | N/A |
| 75:25 % v/v | Solution | Solution | | | | | |
| Acetone | Clear | Clear | Heptane | 0.6 | Slurry | Gum | N/A |
| | Solution | Solution | | | | | |
| Acetonitrile | Clear | Clear | Water | 1.0 | Clear | Hazy | N/A |
| | Solution | Solution | | | Solution | | |
| Ethanol | Clear | Clear | Heptane | 1.0 | Clear | Clear | N/A |
| | Solution | Solution | | | Solution | Solution | |
| Ethyl Acetate | Hazy | Clear | Heptane | 0.4 | Slurry | Gum | N/A |
| | | Solution | | | | | |
| Tetrahydrofuran | Clear | Clear | Heptane | 0.4 | Slurry | Gum | N/A |
| | Solution | Solution | | | | | |

### Benzoic Acid

The following observations were made using benzoic acid as the counterion in Method A. Crystalline solids were obtained from thermal cycling and anti-solvent addition from three of the experiments. One XRPD pattern was observed (osanetant benzoic acid Form 1) which was consistent across the samples. There was no change in form after drying at 40° C. and 40° C./75% RH. Osanetant benzoic acid Form 1 was also obtained from evaporation of the IPA: water experiment, although there were some small additional peaks present. Results are summarized in Table 31.

TABLE 31

| | | | Anti-Solvent Addition | | | Observation | XRPD | | |
|---|---|---|---|---|---|---|---|---|---|
| | Observation | | Anti- | Volume/ | | 2 Day | | | 40° C./ |
| Solvent | Initial | 1 Day | Solvent | mL | Observation | (total) | Wet | 40° C. | 75% RH |
| 2-Propanol:Water | Clear | Clear | Water | 0.6 | Slurry | Hazy | Form 1 * e | N/A | N/A |
| 75:25 % v/v | Solution | Solution | | | | | | | |
| Acetone | Clear | Clear | Heptane | 1.0 | Hazy | Slurry | Form 1 | Form 1 | Form 1 |
| | Solution | Solution | | | | | | | |
| Acetonitrile | Clear | Clear | Water | 1.0 | Slurry | Hazy | N/A | N/A | N/A |
| | Solution | Solution | | | | | | | |
| Ethanol | Clear | Clear | Heptane | 1.0 | Clear | Clear | N/A | N/A | N/A |
| | Solution | Solution | | | Solution | Solution | | | |
| Ethyl Acetate | Clear | Clear | Heptane | 0.6 | Slurry | Slurry | Form 1 | Form 1 | Form 1 |
| | Solution | Solution | | | | | | | |
| Tetrahydrofuran | Clear | Clear | Heptane | 0.8 | Slurry | Slurry | Form 1 | Form 1 | Form 1 |
| | Solution | Solution | | | | | | | |

* = Additional Peaks;
e = evaporation

An X-ray powder diffraction (XRPD) pattern for osanetant benzoic acid Form 1 is shown in FIG. 25. Select peaks which can be used to characterize osanetant benzoic acid Form 1 include one or more of 11.3°2θ±0.2°2θ, 12.7°2θ±0.2°2θ, 15.9°2θ±0.2°2θ, 18.8°2θ±0.2°2θ, 20.3°2θ±0.2°2θ, or 21.2°2θ±0.2°2θ.

The osanetant benzoic acid Form 1 solids consisted of small aggregated crystals. TG analysis indicated that there was a loss of 2.4 wt. % up to 100° C., followed by a loss of 17.8 wt. % up to 250° C. DSC analysis shows a small endothermic event with a peak at 65.6° C. and a broad overlapping endothermic events with a peak at ca. 105° C. There were no further significant thermal events. The simultaneous TG/DSC of osanetant benzoic acid Form 1 is shown in FIG. 36.

Further data on osanetant benzoic acid Form 1 is shown in Table 32.

TABLE 32

| XRPD | ¹H NMR | | Simultaneous TG/DSC | | |
|---|---|---|---|---|---|
| Pattern | Solvent | Counterion | TG - Mass Losses | DSC - Thermal Events | PLM |
| Form 1 | Trace Solvent | <1 eq. - Little | 2.4 wt. % up to | Endothermic event | Small |
| | | Peak Shift | 100° C. | onset 58.5° C. | Aggregated |
| | | | 17.8 wt. % | (peak at 65.6° C.), | |
| | | | up to 250° C. | Endothermic event onset 84.3° C. | |
| | | | | (Peak at 99.6° C.) | |

### Succinic Acid

The following observations were made using succinic acid as the counterion in Method A. Amorphous material was obtained from ethyl acetate. The material was gel-like. There was no change on drying at 40° C. and 40° C./75% RH. Results are summarized in Table 33.

TABLE 33

| | | | Anti-Solvent Addition | | | Observation | XRPD | | |
|---|---|---|---|---|---|---|---|---|---|
| | Observation | | Anti- | Volume/ | | 2 Day | | | 40° C./ |
| Solvent | Initial | 1 Day | Solvent | mL | Observation | (total) | Wet | 40° C. | 75% RH |
| 2-Propanol:Water | Clear | Clear | Water | 1.0 | Slurry | Gum | N/A | N/A | N/A |
| 75:25 % v/v | Solution | Solution | | | | | | | |
| Acetone | Clear | Clear | Heptane | 0.6 | Slurry | Gum | N/A | N/A | N/A |
| | Solution | Solution | | | | | | | |
| Acetonitrile | Clear | Clear | Water | 1.0 | Clear | Clear | N/A | N/A | N/A |
| | Solution | Solution | | | Solution | Solution | | | |
| Ethanol | Clear | Clear | Heptane | 1.0 | Slurry | Clear | N/A | N/A | N/A |
| | Solution | Solution | | | | Solution | | | |
| Ethyl Acetate | Clear | Slurry | N/A | N/A | N/A | Gel Like | Amorphous | Amorphous | Amorphous |
| | Solution | | | | | | | | |
| Tetrahydrofuran | Clear | Clear | Heptane | 0.4 | Slurry | Gum | N/A | N/A | N/A |
| | Solution | Solution | | | | | | | |

### Adipic Acid

The following observations were made using adipic acid as the counterion in Method A. No solids or slurries were obtained from the adipic acid experiments. Results are summarized in Table 34.

TABLE 34

| | | | Anti-Solvent Addition | | | Observation | |
|---|---|---|---|---|---|---|---|
| | Observation | | Anti- | Volume/ | | 2 Day | XRPD |
| Solvent | Initial | 1 Day | Solvent | mL | Observation | (total) | Wet |
| 2-Propanol:Water | Clear | Clear | Water | 0.6 | Slurry | Gum | N/A |
| 75:25 % v/v | Solution | Solution | | | | | |
| Acetone | Clear | Clear | Heptane | 0.4 | Slurry | Clear | N/A |
| | Solution | Solution | | | | Solution | |
| Acetonitrile | Clear | Clear | Water | 1.0 | Clear | Clear | N/A |
| | Solution | Solution | | | Solution | Solution | |
| Ethanol | Clear | Clear | Heptane | 1.0 | Slurry | Gum | N/A |
| | Solution | Solution | | | | | |
| Ethyl Acetate | Clear | Clear | Heptane | 0.4 | Slurry | Clear | N/A |
| | Solution | Solution | | | | Solution | |
| Tetrahydrofuran | Clear | Clear | Heptane | 0.4 | Slurry | Gum | N/A |
| | Solution | Solution | | | | | |

### Acetic Acid

The following observations were made using acetic acid as the counterion in Method A. Solids were obtained from three of the experiments after anti-solvent addition and thermal cycling. A new XRPD pattern, pattern 1 was obtained from the experiments, which changed form on drying at 40° C. to Pattern 2. Pattern 2 was retained on drying at 40° C./75% RH. There were however, traces of free osanetant in the XRPD diffractograms of the material from ethyl acetate: heptane and THF: heptane. Patterns 1 and 2 were assigned Forms 1 and 2, respectively. Results are summarized in Table 35.

TABLE 35

| | | | Anti-Solvent Addition | | | Observation | XRPD | | |
|---|---|---|---|---|---|---|---|---|---|
| | Observation | | Anti- | Volume/ | | 2 Day | | | 40° C./ |
| Solvent | Initial | 1 Day | Solvent | mL | Observation | (total) | Wet | 40° C. | 75% RH |
| 2-Propanol:Water | Clear | Clear | Water | 1.0 | Clear | Clear | N/A | N/A | N/A |
| 75:25 % v/v | Solution | Solution | | | Solution | Solution | | | |
| Acetone | Clear | Clear | Heptane | 0.8 | Slurry | Slurry | Form 1 | Form 2 | Form 2 |
| | Solution | Solution | | | | | | | |
| Acetonitrile | Clear | Clear | Water | 1.0 | Clear | Clear | N/A | N/A | N/A |
| | Solution | Solution | | | Solution | Solution | | | |
| Ethanol | Clear | Clear | Heptane | 1.0 | Clear | Clear | N/A | N/A | N/A |
| | Solution | Solution | | | Solution | Solution | | | |
| Ethyl Acetate | Clear | Clear | Heptane | 0.6 | Slurry | Slurry | Form 1* | Form 2* | Form 2* |
| | Solution | Solution | | | | | | | |
| Tetrahydrofuran | Clear | Clear | Heptane | 0.8 | Slurry | Slurry | Form 1* | Form 2* | Form 2* |
| | Solution | Solution | | | | | | | |

*= Contains Freebase

The X-ray powder diffraction (XRPD) pattern for osanetant acetic acid Form 1 is shown in FIG. 26. Select peaks which can be used to characterize osanetant acetic acid Form 1 include one or more of 7.9°2θ±0.2°2θ, 11.3°2θ±0.2°2θ, 17.8°2θ±0.2°2θ, 18.6°2θ±0.2°2θ, 19.3°2θ±0.2°2θ, or 20.4°2θ±0.2°2θ.

The osanetant Form 2 was determined to be a free base, solids consisted of small aggregated crystals. The X-ray powder diffraction (XRPD) pattern for osanetant free base Form 2 is shown in FIG. 27. Select peaks which can be used to characterize osanetant free base Form 2 include one or more of 11.1°2θ±0.2°2θ, 12.0°2θ±0.2°2θ, 16.0°2θ±0.2°2θ, 17.2°2θ±0.2°2θ, 18.3°2θ±0.2°2θ, or 19.0°2θ±0.2°2θ.

TG analysis of osanetant acetic acid Form 2 indicated that there was a loss of 3.4 wt. % on heating to 125° C. and a further gradual loss of 4.4 wt. % on heating to 265° C. DSC analysis indicated that there was an endothermic event with onset 138.3° C. (peak at 145.9° C.). The simultaneous TG/DSC of osanetant acetic acid Form 2 is shown in FIG. 37.

Further data on osanetant acetic acid Form 2 is shown in Table 36.

TABLE 36

| | ¹H NMR | | Simultaneous TG/DSC | | |
|---|---|---|---|---|---|
| XRPD Pattern | Solvent | Counterion | TG - Mass Losses | DSC - Thermal Events | PLM |
| Form 2 | Trace | 1.5 eq. - Broad | 3.4 wt. % up to | Endothermic event | Small |
| | Solvent | Peak at 12 ppm | 125° C., 4.4 wt. % | onset 138.3° C. (Peak at | Aggregated |
| | | | up to 265° C. | 145.9° C.) | |
| Form 2 | <0.1 eq. | 1.3 eq. - Broad | 5.7 wt. % up to | Endothermic event | N/A |
| | THF | Peak at 12 ppm | 125° C., 5.2 wt. % | onset 140.4° C. (Peak at | |
| | | | up to 265° C. | 145.5° C.) | |

The following crystalline salt forms were stable after drying at 40° C./75% RH: osanetant naphthalene-2-sulfonic acid Forms 1, 2, 3 and 4, osanetant benzenesulfonic acid Forms 1 and 3, osanetant p-toluenesulfonic Form 1, osanetant phosphoric acid Forms 3 and 4, osanetant benzoic acid Form 1, and osanetant acetic acid Form 2.

Based on the results of the above example, the following osanetant salts were scaled up for further characterization: osanetant naphthalene-2-sulfonic acid Form 3 (non solvated, good thermal profile), osanetant benzenesulfonic acid Form 3 (non solvated, good thermal profile), and osanetant p-toluenesulfonic acid Form 1 (stable at 40° C./75% RH). Further data is shown in Table 37.

TABLE 37

| | ¹H NMR | | Simultaneous TG/DSC | |
|---|---|---|---|---|
| XRPD Pattern | Solvent | Counterion | TG - Mass Losses | DSC - Thermal Events |
| p- | 0.05 eq. | Approx. 1 eq | 2.4 wt. % up to 120° C., | Broad Endothermic event (peak |
| toluenesulfonic | IPA | | 10.8 wt. % up to 260° C. | at 97.4° C.), Endothermic event |
| Form 1 | | | | onset 156.3° C. (Peak at |
| | | | | 163.4° C.) |
| naphthalene-2- | Trace | 0.9 eq. | 0.9 wt. % up to 150° C., | Endothermic event onset |
| sulfonic acid | Solvent | | 9.6 wt. % up to 260° C. | 170.5° C. (Peak at |
| Form 3 | | | | 175.5° C.) |
| benzenesulfonic | Trace | <1 eq. | 0.6 wt. % up to 155° C., | Endothermic event onset |
| acid Form 3 | Solvent | | 11.3 wt. % up to 265° C. | 176.8° C. (Peak at |
| | | | | 181.0° C.) |

### Example 4A

Process for Preparing Osanetant p-Toluenesulfonic Acid

The p-toluene sulfonic acid salt of osanetant was prepared on a 6 g scale, to provide material as input to the polymorph screen. The following procedure was carried out for the scale up of the p-toluene sulfonic acid salt: 6 g of osanetant was suspended in 90 mL of ethyl acetate. 2.016 g of p-toluenesulfonic acid monohydrate was added directly to the osanetant suspension. A clear solution was observed. The experiment was thermally cycled as follows with agitation: 4 h at 40° C.; 4 h at ambient temperature. After ca. 16 h, a slurry was observed. The slurry was sampled (a)—ca. 0.5 mL was filtered by centrifugation. The solids were analyzed by XRPD. The slurry was returned to thermal cycling for ca. 24 h. The slurry was sampled (b)—ca. 0.5 mL was filtered by centrifugation. The solids were analyzed by XRPD. The slurry was returned to thermal cycling for ca. 24 h. The slurry was sampled (c)—ca. 0.5 mL was filtered by centrifugation. The solids were analyzed by XRPD. The slurry was agitated at 37° C. for ca. 72 h. The slurry was sampled (d)—ca. 0.5 mL was filtered by centrifugation. The solids were analyzed by XRPD. The solids were dried at 40° C. under vacuum for ca. 16 h. The dried solids were analyzed by XRPD, TG/DSC and 1H NMR. The slurry was isolated by Buchner filtration using 70 mm Ø grade 1 Whatman filter paper, and fast filtration (<30 s) was carried out. The isolated solids were dried at 40° C. under vacuum for ca. 20 h. The dried solids were analyzed by XRPD, TG/DSC, and KF. The XRPD indicated that the solids corresponded to osanetant p-toluenesulfonic acid salt Form 6 (anhydrous form).

Amorphous osanetant p-toluenesulfonic acid salt: The solubility of the p-toluene sulfonic acid salt was assessed in solvents suitable for lyophilization, then any solutions were frozen, lyophilized and the solids assessed for crystallinity. The following procedure was carried out to prepare amorphous p-toluene sulfonic acid salt: 3 g of the p-toluene sulfonic acid salt was dissolved in 300 mL of 1,4-dioxane with gentle heating (10 mg/mL). The solution was split between 60 vials (approximately 50 mg per vial). The solution was frozen, then lyophilized. After 48 h, lyophilization was complete, and the solids were analysed by XRPD.

10 mL of 1,4-dioxane was added to each of the p-toluene sulfonic acid vials—5 mg/mL. Complete dissolution was not observed, a thin shiny slurry was observed. The thin slurries were combined, and an additional ca. 200 mL of 1,4 dioxane was added. The slurry was heated gently for ca. 30 min, however dissolution was not observed. The thin slurry was Buchner filtered (90 mm Ø GF filter paper). The clear solution was split between two crystallization dishes and frozen in a freezer at ca. −20° C., then was lyophilized. XRPD analysis was carried out on the lyophilized material from each of the two crystallization dishes.

### Example 4B

Process for Preparing Osanetant p-Toluenesulfonic Acid Form 1

### Method B

The following procedure was carried out for the scale up of osanetant p-toluenesulfonic acid Form 1. Osanetant (500 mg) was dissolved in 7.5 mL of ethyl acetate at 40° C. The experiment was stirred throughout with magnetic stirring. A clear solution was observed after 20 min at 40° C. 168 mg of toluenesulfonic acid monohydrate was added directly to the osanetant solution. The acid vial was washed with 0.5 mL of ethyl acetate which was added to the experiment. A clear solution was observed. The experiment was thermally cycled as follows: 40° C. to 5° C. at a rate of 0.1° C./min; 1 h at 5° C.; 5 to 40° C. at a rate of 0.1° C./min; 1 h at 40° C.

On the second heat cycle, a slurry was observed at 20° C. The experiment was cooled to 5° C. at a rate of 0.1° C./min and stirred at 5° C. for 18 h. The slurry was isolated be Buchner filtration: 42.5 mm Ø grade 1 Whatman filter paper; fast Filtration (<30 s); the cake was washed with 1 mL of ethyl acetate; the isolated solids were dried at 40° C. under vacuum for 72 h.

XRPD analysis indicated that the wet solids were consistent with osanetant p-toluenesulfonic acid Form 1. There was no change on drying at 40° C. under vacuum. XRPD analysis indicated that osanetant p-toluenesulfonic acid Form 1 was isolated from the experiment, with no change in form on drying. An isolated yield of 87% was obtained based on a osanetant p-toluenesulfonic acid Form 1 monohydrate. The mother liquor had a concentration of 0.96 mg/mL. The theoretical yield was 99.1%. HPLC analysis indicated that the solids contained 0.07% w/w impurities. PLM analysis indicated that the solids consisted of small aggregated crystals that were poorly birefringent. The FT-IR spectrum indicated that water was present. TG analysis indicated that there was a loss of 2.3 wt. % (1 mol eq. water) on heating to 120° C. There was a further loss of 10.3 wt. % on heating to 280° C. DSC analysis indicated that there was a broad endothermic event related to the first mass loss with an onset of 87.6° C. (peak at 106.2° C.). There was an endothermic event with onset 153.4° C. (peak at 159.7° C.), which was likely the melt event, as it was before the second mass loss. ¹H NMR analysis indicated that one equivalent of toluenesulfonic acid was present. Trace ethyl acetate was present. There were signal shifts in comparison to osanetant, which was indicative of salt formation. KF analysis indicated that the material had a moisture content of 2.2 wt. %. This was consistent with TG analysis and showed that osanetant p-toluenesulfonic acid Form 1 was a monohydrate.

### Hydration Study

20 mg of osanetant p-toluenesulfonic acid Form 1 was stirred in the appropriate ethanol: water mixtures for 24 h at 25° C. Where clear solutions were observed after 1 h at 25° C., 0.5 mL of liquor was removed, and additional solid was added until a slurry was maintained. After 24 h total, the experiments were isolated by centrifugation, and the solids were analyzed by XRPD. XRPD analysis of the solids after stirring at 25° C., showed that there was no change in form. Further data is shown in Table 38.

TABLE 38

| | | Volume | | | Estimated | | |
|---|---|---|---|---|---|---|---|
| Calc | Ethanol:Water | Solvent/ | Initial | 1 h | Additional | 24 h | |
| Aw | % v/v | mL | Observation | Observation | Solid/mg | Observation | XRPD |
| 0.257 | 98.4:1.6 | 1 | White | Clear | 20 | White | Form 1 |
| | | | Slurry | Solution | | Slurry | |
| 0.507 | 92.9:7.1 | 0.4 | White | White | N/A | White | Form 1 |
| | | | Slurry | Slurry | | Slurry | |
| 0.745 | 63.6:36.4 | 0.2 | White | White | N/A | White | Form 1 |
| | | | Slurry | Slurry | | Slurry | |

### Disproportionation Study

Osanetant p-toluenesulfonic acid Form 1 (10 mg) was stirred in 1 mL of water for 24 h at 25° C. After 24 h total, a white slurry was observed and was isolated by centrifugation. The solids were analyzed by XRPD. The pH of the filtered liquor was measured at 5.63. XRPD analysis indicated that there was no change in form after stirring in water for 24 h at 25° C. The salt did not disproportionate and osanetant p-toluenesulfonic acid Form 1 was observed.

### Example 5A

### Process for Preparing Osanetant Benzenesulfonic Acid Form 3

### Method C

The following procedure was carried out for the scale up of osanetant benzenesulfonic acid Form 3. Osanetant (500 mg) was dissolved in 7.5 mL of acetone at 40° C. The experiment was stirred throughout with magnetic stirring. A clear solution was observed after 20 min at 40° C. 39.7 mg of benzenesulfonic acid was added directly to the osanetant solution. The acid vial was washed with 0.5 mL of acetone which was added to the experiment. A clear solution was observed. The experiment was thermally cycled as follows: 40° C. to 5° C. at a rate of 0.1° C./min; 1 h at 5° C.; 5 to 40° C. at a rate of 0.1° C./min; 1 h at 40° C. On the second heat cycle, a slurry was observed at 20° C. The experiment was cooled to 5° C. at a rate of 0.1° C./min and stirred at 5° C. for 18 h. The slurry was isolated be Buchner filtration: 42.5 mm Ø grade 1 Whatman filter paper; fast Filtration (<30 s); the cake was washed with 1 mL of acetone. The isolated solids were dried at 40° C. under vacuum for 72 h. XRPD analysis indicated that the wet solids were consistent with osanetant benzenesulfonic acid Form 3. There was no change on drying at 40° C. under vacuum. XRPD analysis indicated that osanetant benzenesulfonic acid Form 3 was isolated from the experiment, with no change in form on drying.

PLM analysis indicated that the solids consisted of small aggregated lath-like crystals that were poorly birefringent. The FT-IR spectrum indicated that there were shifts in the signal positions in comparison to osanetant. TG analysis indicated that there was a loss of 0.1 wt. % on heating to 150° C. There was a further loss of 10.4 wt. % on heating to 290° C. DSC analysis indicated that there was an endothermic event with an onset of 179.4° C. (peak at 184.7° C.), which occurred before the mass loss.

DSC analysis indicated that on the first heat cycle, there was an endothermic event with onset 180.2° C. (peak at 183.4° C.). On the cool cycle, there was a glass transition with a mid-point of 35.1° C. On the second heat cycle, there was a glass transition with a mid-point at 45.0° C. ¹H NMR analysis indicated that there was slight excess of benzenesulfonic acid present—1.2 mol eq. Trace acetone was present. There were signal shifts in comparison to osanetant, which was indicative of salt formation.

### Hydration Study

Osanetant benzenesulfonic acid Form 3 (20 mg) was stirred in the appropriate ethanol: water mixtures for 24 h at 25° C. Where clear solutions were observed after 1 h at 25° C., 0.5 mL of liquor was removed from the Aw 0.257 sample, and additional solid was added until a slurry was maintained. After 24 h total, the experiments were isolated by centrifugation, and the solids were analyzed by XRPD. XRPD analysis of the solids after stirring at 25° C., showed that at Aw 0.507 and above, there was conversion to osanetant benzenesulfonic acid Form 1. Analysis from Example 3 indicated that osanetant benzenesulfonic acid Form 1 was a monohydrate. XRPD analysis of the solids after stirring at 25° C., showed that at Aw 0.507 and above, there was conversion to osanetant benzenesulfonic acid Form 1. Further data is shown in Table 39.

TABLE 39

| | | Volume | | | Estimated | | |
|---|---|---|---|---|---|---|---|
| Calc | Ethanol:Water | Solvent/ | Initial | 1 h | Additional | 24 h | |
| Aw | % v/v | mL | Observation | Observation | Solid/mg | Observation | XRPD |
| 0.257 | 98.4:1.6 | 1.0 | White | Clear | 20 | White | Form 3 |
| | | | Slurry | Solution | | Slurry | |
| 0.507 | 92.9:7.1 | 0.4 | White | Clear | 30 | White | Form 1 |
| | | | Slurry | Solution | | Slurry | |
| 0.745 | 63.6:36.4 | 0.2 | White | White | N/A | White | Form 1 |
| | | | Slurry | Slurry | | Slurry | |

### Disproportionation Study

Osanetant benzenesulfonic acid Form 3 (10 mg) was stirred in 1 mL of water for 24 h at 25° C. After 24 h total, a white slurry was observed and was isolated by centrifugation. The solids were analyzed by XRPD. The pH of the filtered liquor was measured (pH=5.09). There was a change in form from osanetant benzenesulfonic acid Form 3 to osanetant benzenesulfonic acid Form 1 after stirring in water for 24 h at 25° C. XRPD analysis indicated that there the material converted to osanetant benzenesulfonic acid Form 1. Analysis from the primary salt screen indicated that osanetant benzenesulfonic acid Form 1 was a monohydrate.

Example 5B

### Process for Preparing Osanetant Benzenesulfonic Acid Form 3 and Amorphous Osanetant Benzenesulfonic Acid

### Method D

The benzene sulfonic acid salt of osanetant was prepared on a 6 g scale, to provide material as input to the polymorph screen. The following procedure was carried out for the scale up of the besylate salt: 6 g of osanetant was suspended in 90 mL of acetone. 1.676 g of benzenesulfonic acid was added directly to the osanetant suspension. A clear solution was observed. The experiment was thermally cycled as follows: 4 h at 40° C.; 4 h at ambient temperature. After ca. 16 h, a slurry was observed. The slurry was sampled (a)—ca. 0.5 mL was filtered by centrifugation. The solids were analysed by XRPD. The slurry was returned to thermal cycling for ca. 24 h. The slurry was sampled (b)—ca. 0.5 mL was filtered by centrifugation. The solids were analysed by XRPD. The slurry was isolated by Buchner filtration: 70 mm Ø grade 1 Whatman filter paper; fast filtration (<30 s) was carried out. The isolated solids were dried at 40° C. under vacuum for ca. 18 h. The dried solids were analysed by XRPD, TG/DSC, and KF. XRPD indicated the solids corresponded to osanetant benzene sulfonic acid salt Form 3 (anhydrous form).

Amorphous osanetant benzene sulfonic acid salt: The solubility of the benzene sulfonic acid salt was assessed in solvents suitable for lyophilization, then any solutions were frozen, lyophilized and the solids assessed for crystallinity. The following procedure was carried out for preparation of amorphous osanetant benzene sulfonic acid salt: 3 g of the benzene sulfonic acid salt was dissolved in 650 mL of 1,4-dioxane with gentle heating (4.6 mg/mL). The solution was split between 60 vials (approximately 50 mg per vial). The solution was frozen, then lyophilized. After 72 h, lyophilization was complete and the solids were analyzed by XRPD.

### Example 5C

### Process for Preparing Osanetant Benzenesulfonic Acid Salt Form 1

### Method E

500 mg of osanetant benzene sulfonic acid Form 3 was weighed in a scintillation vial. 10 mL of water was added, and the suspension was stirred at room temperature (ca. 20° C.) for ca 18 hours. A 0.5 mL aliquot of the slurry was taken, and the solids were isolated by centrifugation (subsample a). The solid was analyzed by XRPD and found to be a mixture of osanetant benzene sulfonic acid salt Form 1 and osanetant benzene sulfonic acid salt Form 3. An additional 10 mL of water was added, and the suspension stirred for a further 4 hours. A 0.5 mL aliquot of the slurry was taken, and the solids were isolated by centrifugation (subsample b). The solid was analyzed by XRPD. The slurry was isolated by Buchner filtration using 42.5 mm Ø grade 1 Whatman filter paper. The isolated solids were dried at 40° C. under vacuum for ca. 20 h. The dried solids were analyzed by XRPD and found to be osanetant benzene sulfonic acid salt Form 1.

### Example 6

### Comparative Studies

Osanetant p-toluenesulfonic acid Form 1, osanetant benzene sulfonic acid Form 3, osanetant naphthalene-2-sulfonic acid Form 3, and osanetant phosphoric acid Form 7 were compared. Osanetant phosphoric acid Form 7 was obtained when preparation of osanetant phosphoric acid Form 4 was repeated and found to be not reproducible, and Form 7 was determined to be a mixture of hydrates).

TABLE 40

| | | | p-Toluene sulfonic acid | | Benzene sulfonic acid | |
|---|---|---|---|---|---|---|
| | | | (pTSA) | | (Besylate) | |
| | | | pTSA | pTSA | Besylate | Besylate |
| Analysis | | | Form 1 | Form 6 | Form 3 | Form 1 |
| Input Form | | | (Hydrate) | (Anhydrous) | (Anhydrous) | (Hydrate) |
| Slurry at | Ethanol | Aw* 0.257 | pTSA | N/A | Besylate | N/A |
| 25° C. | water | | Form 1 | | Form 3 | |
| | | Aw 0.507 | pTSA | N/A | Besylate | N/A |
| | | | Form 1 | | Form 1 | |
| | | Aw 0.745 | pTSA | N/A | Besylate | N/A |
| | | | Form 1 | | Form 1 | |
| | Water | Liquor pH | 5.63 | N/A | 5.09 | N/A |
| | | XRPD | pTSA | N/A | Besylate | N/A |
| | | | Form 1 | | Form 1 | |
| 7 Day | Ambient | Observation | White | White | White | White |
| Stability | Light | | Solids | Solids | Solids | Solids |
| | | XRPD | pTSA | pTSA | Besylate | Besylate |
| | | | Form 1 | Form 6 | Form 3 | Form 1 |
| | | HPLC | 0.06 | 0.11 | 0.05 | 0.00 |
| | | Impurity | | | | |
| | | Content / | | | | |
| | | % w/w | | | | |
| | 40° C./ | Observation | White | White | White | White |
| | 75% RH | | Solids | Solids | Solids | Solids |
| | | XRPD | pTSA | pTSA Form | Besylate | Besylate |
| | | | Form 1 | 6 + pTSA | Form 3 | Form 1 |
| | | | | Form 1 | | |
| | | HPLC | 0.18 | 0.11 | 0.06 | 0.05 |
| | | Impurity | | | | |
| | | Content / | | | | |
| | | % w/w | | | | |
| | 80° C. | Observation | White | White | White | White |
| | | | Solids | Solids | Solids | Solids |
| | | XRPD | pTSA | pTSA | Besylate | Besylate |
| | | | Form 1 | Form 6 | Form 3 | Form 1 |
| | | HPLC | 1.46 | 0.88 | 0.24 | 0.06 |
| | | Impurity | | | | |
| | | Content / | | | | |
| | | % w/w | | | | |
| 24 h | pH 1.2 | Concentration / | 0.1473 | 0.0458 | 0.2163 | 0.1711 |
| Solubility | | mg/mL | | | | |
| at 37° C. | | XRPD | pTSA | pTSA | Besylate | Besylate |
| | | | Form 1 | Form 1 | Form 1 | Form 1 |
| | pH 4.5 | Concentration / | 0.1197 | 0.0630 | 0.2324 | 0.2083 |
| | | mg/mL | | | | |
| | | XRPD | pTSA | pTSA | Besylate | Besylate |
| | | | Form 1 | Form 1 | Form 1 | Form 1 |
| | pH 6.8 | Concentration / | 0.0849 | 0.0356 | 0.3637 | 0.0023 |
| | | mg/mL | | | | |
| | | XRPD | pTSA | pTSA | Besylate | Besylate |
| | | | Form 1 | Form 1 | Form 3 | Form 1 |
| | | | | | | and |
| | | | | | | Osanetant |
| | | | | | | Free base |

Aw = calculated water activity

Osanetant p-toluenesulfonic acid Form 1 was a stable monohydrate which dehydrated above 85° C., to osanetant p-toluenesulfonic acid Form 5, which hydrated at ambient conditions to Form 1. There was an increase in impurities on 7-day stability at 80° C., however the form was retained. Osanetant p-toluenesulfonic acid Form 1 was slightly hygroscopic with 0.2 wt. % gain at 80% RH. Solubility in water and pH buffers was low.

Osanetant benzene sulfonic acid Form 3 was an anhydrous form which retained form on 7-day stability. A slight increase in impurities on 7-day stability at 80° C. was found. Osanetant benzene sulfonic acid Form 3 converted to osanetant benzene sulfonic acid Form 1, a hydrated form, in solutions with high water activity. The thermodynamic solubility in pH buffers was low, and Form 3 converted to Form 1. Osanetant benzene sulfonic acid Form 1 had a gradual loss of water during TG analysis.

Osanetant phosphoric acid Form 7 was a hydrated form. Extensive polymorphism was observed, likely due to multiple hydrated forms. Good solid chemical stability was observed. Osanetant phosphoric acid Form 7 had very high solubility in water and buffered systems at pH 1.2 and 4.5. Disproportionation was observed in pH 6.8 buffer.

Osanetant naphthalene-2-sulfonic acid Form 3 was a stable solid form with similar chemical stability as the osanetant p-toluenesulfonic acid forms, with an increase in impurities on 7-day stability at 80° C. Low solubility was observed in buffered systems.

Based on the 7-day stability results, the phosphate salt had the best chemical stability, however there was a loss in crystallinity at elevated temperature, likely due to dehydration. The three sulfonic acid salts each had an increase in impurities at 80° C. on 7-day stability, but the benzene sulfonic acid salt unexpectedly had the lowest impurity increase. Although the chemical stability of the osanetant benzene sulfonic acid salt Form 3 was good, it converted to osanetant benzene sulfonic acid salt form Form 1, a hydrate, in many of the experiments. Also, surprisingly, osanetant naphthalene-2-sulfonic acid Form 3 was more hygroscopic in comparison to the p-toluene sulfonic acid and the benzene sulfonic acid salts that were tested in this experiment. Thus, osanetant benzene sulfonic acid salt Form 1 and osanetant p-toluene sulfonic acid salt Form 1 were identified as advantageous salts.

### Example 7

### Long Term Stability Studies

Osanetant p-toluenesulfonic acid Form 1 and osanetant benzenesulfonic acid Form 1 were tested for long-term stability across a number of different conditions, as detailed below.

Thermal stability of solids at 60° C.—Osanetant p-toluenesulfonic acid Form 1 and osanetant benzenesulfonic acid Form 1 were stored in a darkened oven at 60° C. in glass vials. Samples were taken after T=1, 2, 4, 7, and 14 days storage. An additional sample was taken after 1 month due to lack of degradation observed. The purity of the resulting material is described in Table 41:

TABLE 41

| | | T = 1 | T = 2 | T = 4 | T = 7 | T = 14 | T = 1 |
|---|---|---|---|---|---|---|---|
| | Control | day | day | day | day | day | month |
| Salt | Purity | Purity | Purity | Purity | Purity | Purity | Purity |
| p-toluene- | 99.8 | 99.7 | 99.7 | 99.7 | 99.6 | 99.7 | 99.5 |
| sulfonic acid | | | | | | | |
| Form 1 | | | | | | | |
| benzenesulfonic | 99.9 | 99.9 | 99.9 | 99.9 | 99.9 | 100.0 | 100.0 |
| acid Form 1 | | | | | | | |

Purity = 100-total other substances, calculated with respect to osanetant content

Acid Stability at Ambient Temperature-Osanetant p-toluenesulfonic acid Form 1 and osanetant benzenesulfonic acid Form 1 were prepared at an osanetant content 10 times the normal working concentration then added to HPLC vials containing 2M HCl and stored in in the dark at ambient laboratory conditions. Samples were diluted to 2 mg/mL for analysis and were taken after T=1, 2, 4, 7, and 14 days storage. An additional sample was taken after 1 month due to lack of degradation observed. The purity of the resulting material is described in Table 42:

TABLE 42

| | | T = 1 | T = 2 | T = 4 | T = 7 | T = 14 | T = 1 |
|---|---|---|---|---|---|---|---|
| | Control | day | day | day | day | day | month |
| Salt | Purity | Purity | Purity | Purity | Purity | Purity | Purity |
| p-toluene- | 99.8 | 99.8 | 99.8 | 99.9 | 99.6 | 99.0 | 98.0 |
| sulfonic acid | | | | | | | |
| Form 1 | | | | | | | |
| benzenesulfonic | 99.9 | 100.0 | 100.0 | 99.9 | 99.5 | 99.3 | 98.5 |
| acid Form 1 | | | | | | | |

Purity = 100-total other substances, calculated with respect to osanetant content

Acid Stability at 60° C.—Osanetant p-toluenesulfonic acid Form 1 and osanetant benzenesulfonic acid Form 1 were prepared at an osanetant content 10 times the normal working concentration then added to HPLC vials containing 2M HCl and stored in a darkened oven at 60° C. Samples were diluted to 2 mg/mL for analysis and were taken after T=1, 2, and 4 days storage. The study was stopped after day 4 as purity dropped below 90%, as shown in Table 43:

TABLE 43

| | | T = 1 | T = 2 | T = 4 |
|---|---|---|---|---|
| | Control | day | day | day |
| Salt | Purity | Purity | Purity | Purity |
| p-toluene- | 99.8 | 94.8 | 89.0 | 80.5 |
| sulfonic acid | | | | |
| Form 1 | | | | |
| benzenesulfonic | 99.9 | 94.7 | 89.2 | 81.0 |
| acid Form 1 | | | | |

Purity = 100-total other substances, calculated with respect to osanetant content

Base at Ambient Temperature—Osanetant p-toluenesulfonic acid Form 1 and osanetant benzenesulfonic acid Form 1 were prepared at an osanetant content 10 times the normal working concentration then added to HPLC vials containing 2M NaOH and stored in the dark at ambient laboratory conditions. Samples were diluted to 2 mg/mL for analysis and were taken after T=1, 2, 4, 7, and 14 days storage. An additional sample was taken after 1 month due to lack of degradation observed. The purity of the resulting material is described in Table 44:

TABLE 44

| | | T = 1 | T = 2 | T = 4 | T = 7 | T = 14 | T = 1 |
|---|---|---|---|---|---|---|---|
| | Control | day | day | day | day | day | month |
| Salt | Purity | Purity | Purity | Purity | Purity | Purity | Purity |
| p-toluene- | 99.8 | 99.8 | 99.8 | 99.8 | 99.8 | 99.8 | 99.7 |
| sulfonic acid | | | | | | | |
| Form 1 | | | | | | | |
| benzenesulfonic | 99.9 | 99.9 | 99.9 | 99.9 | 99.9 | 100.0 | 99.9 |
| acid Form 1 | | | | | | | |

Purity = 100-total other substances, calculated with respect to osanetant content

Base at 60° C.—Osanetant p-toluenesulfonic acid Form 1 and osanetant benzenesulfonic acid Form 1 were prepared at an osanetant content 10 times the normal working concentration then added to HPLC vials containing 2M NaOH and stored in a darkened oven at 60° C. Samples were diluted to 2 mg/mL for analysis and were taken after T=1, 2, 4, 7, and 14 days storage. An additional sample was taken after 1 month due to lack of degradation observed. The purity of the resulting material is described in Table 45:

TABLE 45

| | | T = 1 | T = 2 | T = 4 | T = 7 | T = 14 | T = 1 |
|---|---|---|---|---|---|---|---|
| | Control | day | day | day | day | day | month |
| Salt | Purity | Purity | Purity | Purity | Purity | Purity | Purity |
| p-toluene- | 99.8 | 99.6 | 99.2 | 98.4 | 97.1 | 94.7 | 94.0 |
| sulfonic acid | | | | | | | |
| Form 1 | | | | | | | |
| benzenesulfonic | 99.9 | 99.7 | No | 98.5 | 97.3 | 95.5 | 95.8 |
| acid Form 1 | | | data | | | | |

Purity = 100-total other substances, calculated with respect to osanetant content

Oxidation at Ambient Temperature-Osanetant p-toluenesulfonic acid Form 1 and osanetant benzenesulfonic acid Form 1 were prepared at an osanetant content 10 times the normal working concentration then added to HPLC vials containing 6% H2O2 and stored in the dark at ambient laboratory conditions. Samples were diluted to 2 mg/mL for analysis and were taken after T=1, 2, 4, 7, and 14 days storage. An additional sample was taken after 1 month due to lack of degradation observed. The purity of the resulting material is described in Table 46:

TABLE 46

| | | T = 1 | T = 2 | T = 4 | T = 7 | T = 14 | T = 1 |
|---|---|---|---|---|---|---|---|
| | Control | day | day | day | day | day | month |
| Salt | Purity | Purity | Purity | Purity | Purity | Purity | Purity |
| p-toluene- | 99.8 | 99.8 | 99.8 | 99.8 | 99.8 | 99.8 | 99.7 |
| sulfonic acid | | | | | | | |
| Form 1 | | | | | | | |
| benzenesulfonic | 99.9 | 99.9 | 99.9 | 99.9 | 99.8 | 99.8 | 99.5 |
| acid Form 1 | | | | | | | |

Purity = 100-total other substances, calculated with respect to osanetant content

Oxidation at 60° C.—Osanetant p-toluenesulfonic acid Form 1 and osanetant benzenesulfonic acid Form 1 were prepared at an osanetant content 10 times the normal working concentration then added to HPLC vials containing 6% H2O2 and stored in a darkened oven at 60° C. Samples were diluted to 2 mg/mL for analysis and were taken after T=1, 2, 4, 7, and 14 days storage. An additional sample was taken after 1 month due to lack of degradation observed. The purity of the resulting material is described in Table 47:

TABLE 47

| | | T = 1 | T = 2 | T = 4 | T = 7 | T = 14 | T = 1 |
|---|---|---|---|---|---|---|---|
| | Control | day | day | day | day | day | month |
| Salt | Purity | Purity | Purity | Purity | Purity | Purity | Purity |
| p-toluene- | 99.8 | 99.3 | 96.6 | 97.4 | 95.2 | 92.4 | 65.5 |
| sulfonic acid | | | | | | | |
| Form 1 | | | | | | | |
| benzenesulfonic | 99.9 | 91.9 | 97.2 | 94.8 | 93.1 | 76.6 | 77.8 |
| acid Form 1 | | | | | | | |

Purity = 100-total other substances, calculated with respect to osanetant content

### Biological Assays

The crystalline salt forms as described above can be tested in published assays for biological activity, such as, but not limited to, those described in one or more of WO2001095904, WO2002089802, WO2014089019, and WO2015200594.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1-31. (canceled)

32. Crystalline (R)-N-(1-(3-(1-benzoyl-3-(3,4-dichlorophenyl)piperidin-3-yl)propyl)-4-phenylpiperidin-4-yl)-N-methylacetamide benzenesulfonic acid (osanetant benzenesulfonic acid Form 1) characterized by an X-ray powder diffractogram comprising the following peaks: 11.3, 17.9, and 19.3°2θ±0.2°2θ, and optionally one or more additional peaks selected from 14.9, 16.7, and 18.6°2θ±0.2°2θ.

33. The osanetant benzenesulfonic acid Form 1 according to claim 32, characterized by a differential scanning calorimetry (DSC) curve that comprises an endotherm at with an onset of about 147.6° C. (peak at about 156.3° C.).

34. The osanetant benzenesulfonic acid Form 1 according to claim 32, wherein the osanetant benzenesulfonic acid Form 1 is a monohydrate.

35. Crystalline (R)-N-(1-(3-(1-benzoyl-3-(3,4-dichlorophenyl)piperidin-3-yl)propyl)-4-phenylpiperidin-4-yl)-N-methylacetamide benzenesulfonic acid (osanetant benzenesulfonic acid Form 3) characterized by an X-ray powder diffractogram comprising the following peaks: 12.8, 16.4, and 17.4°2θ±0.2°2θ, and optionally one or more additional peaks selected from 18.3, 21.4, and 21.8°2θ±0.2°2θ.

36. The osanetant benzenesulfonic acid Form 3 according to claim 35, characterized by a differential scanning calorimetry (DSC) curve that comprises an endotherm at with an onset of about 176.8° C. (peak at about 181.0° C.).

37. The osanetant benzenesulfonic acid Form 3 according to claim 35, wherein the osanetant benzenesulfonic acid Form 3 is non-solvated.

38. A pharmaceutical composition comprising at least one of crystalline (R)-N-(1-(3-(1-benzoyl-3-(3,4-dichlorophenyl)piperidin-3-yl)propyl)-4-phenylpiperidin-4-yl)-N-methylacetamide benzenesulfonic acid (osanetant benzenesulfonic acid Form 1) or (R)-N-(1-(3-(1-benzoyl-3-(3,4-dichlorophenyl)piperidin-3-yl)propyl)-4-phenylpiperidin-4-yl)-N-methylacetamide benzenesulfonic acid (osanetant benzenesulfonic acid Form 3) in combination with one or more pharmaceutically acceptable excipients.

39. The pharmaceutical composition of claim 38, wherein the composition comprises osanetant benzenesulfonic acid Form 1.

40. The pharmaceutical composition of claim 38, wherein the composition comprises osanetant benzenesulfonic acid Form 3.

41. The pharmaceutical composition of claim 38, wherein at least about 90%, about 95%, about 99%, about 99.5%, about 99.9%, or about 99.99% of the osanetant present in the composition is osanetant benzenesulfonic acid Form 1 or osanetant benzenesulfonic acid Form 3.

42. A method for treating, pretreating, or delaying onset or progression of a condition mediated at least in part by neurokinin 3 receptor antagonism, comprising administering to a patient in need thereof, a therapeutically effective amount of a crystalline (R)-N-(1-(3-(1-benzoyl-3-(3,4-dichlorophenyl)piperidin-3-yl)propyl)-4-phenylpiperidin-4-yl)-N-methylacetamide benzenesulfonic acid (osanetant benzenesulfonic acid Form 1) or (R)-N-(1-(3-(1-benzoyl-3-(3,4-dichlorophenyl)piperidin-3-yl)propyl)-4-phenylpiperidin-4-yl)-N-methylacetamide benzenesulfonic acid (osanetant benzenesulfonic acid Form 3).

43. The method of claim 42, wherein the method treats a condition mediated by neurokinin 3 receptor antagonism.

44. The method of claim 42, comprising administering osanetant benzenesulfonic acid Form 1.

45. The method of claim 42, comprising administering osanetant benzenesulfonic acid Form 3.

46. The method of claim 42, wherein the condition mediated at least in part by neurokinin 3 receptor antagonism is selected from the group consisting of a disease associated with a dysfunction of the dopaminergic system, a disease associated with a dysfunction of the dopaminergic system, a vigilance disorder, an epileptic disease, a neurodegenerative disease, a peripheral disease, a cardiovascular disorder, a rhythm disorder, a respiratory disorder, a disorder of the gastrointestinal system, a stress-related disorder, a disorder of the urinary system, acute or chronic inflammation, a disease of the immune system, schizophrenia, Parkinson's disease, anxiety, Grand Mal, dementia, pain, migraine, hypertension, cardiac insufficiency, asthma, rhinitis, cough, bronchitis, allergies, hypersensitivity, esophageal ulcer, colitis, irritable bowel syndrome (IBS), inflammatory bowel diseases (IBD), acidic secretion, incontinence, neurogenic bladder, rheumatoid arthritis, altered bowel function, androgen dependent acne, androgen-producing tumors, anxiety, atherosclerosis, atresia, anovulation, bladder dysfunction, benign prostatic hyperplasia (BPH), cancer, chronic traumatic encephalopathy, coronary artery disease, dysarthria, dysfunction of the dopaminergic systems, dysfunctional uterine bleeding, dysmenorrhea, dysphagia, eye movement difficulties, follicular maturation arrest, gastrointestinal dysfunction, HAIR-AN syndrome, hirsutism, hot flashes, hyperandrogenism, infertility, inflammation, inflammatory response, insomnia, loss of muscle coordination, loss of muscle function, macular degeneration, male pattern baldness, metastatic prostatic carcinoma, muscle wasting, nausea or vomiting, oculomotor gaze palsy, ovarian hyperthecosis, Parkinson's disease, peripheral vascular disease, polycystic ovary syndrome (PCOS), precocious puberty in boys, pre-eclampsia, acute stress disorder (ASD), post-traumatic stress disorder (PTSD), reduced dopaminergic neuron function, reducing intracranial pressure, reperfusion injury, respiratory depression, respiratory disorders, rheumatoid arthritis, supranuclear gaze palsy, testicular cancer, treating benign prostatic hyperplasia, treating mood disorders, treatment of excess body weight and/or excess body fat, varicose veins, vasomotor symptoms, virilization, and visceral pain.

47. The method of claim 42, wherein the condition mediated at least in part by neurokinin 3 receptor antagonism is categorized as a DSM-5 disorder.

48. The method of claim 46, wherein the condition mediated at least in part by neurokinin 3 receptor antagonism comprises post-traumatic stress disorder (PTSD), acute stress disorder (ASD), schizophrenia, anxiety, or depression.

49. The method of claim 46, wherein the condition mediated at least in part by neurokinin 3 receptor antagonism is vasomotor symptoms or hot flashes.

50. The method of claim 46, wherein the condition mediated at least in part by neurokinin 3 receptor antagonism is metastatic prostatic carcinoma.

51. The method of claim 46, wherein the patient is a human and the effective amount is a daily dose of between 50 and 200 mg per day, about 25 mg per day, about 50 mg per day, about 100 mg per day, about 200 mg per day, 25 mg twice a day (BID), or 100 mg BID.
